(19) United States
(12) Patent Application Publication
(10) Pub. No.: US 2024/0057971 A1
(43) Pub. Date: Feb. 22, 2024

(54) TRANSCRANIAL ULTRASOUND DEVICES AND METHODS

(71) Applicant: Teratech Corporation, Burlington, MA (US)

(72) Inventors: Alice M. Chiang, Wayland, MA (US); Noah Berger, Sudbury, MA (US)

(73) Assignee: Teratech Corporation, Burlington, MA (US)

(21) Appl. No.: 18/196,379

(22) Filed: May 11, 2023

Related U.S. Application Data
(63) Continuation-in-part of application No. 18/090,316, filed on Dec. 28, 2022.
Application No. 18/090,316 is a continuation-in-part of application No. 16/938,515, filed on Jul. 24, 2020.
Application No. 16/938,515 is a continuation-in-part of application No. 16/414,215, filed on May 16, 2019.
(60) Provisional application No. 63/294,307, filed on Dec. 28, 2021.
(60) Provisional application No. 62/878,163, filed on Jul. 24, 2019.
(60) Provisional application No. 62/830,200, filed on Apr. 5, 2019.
(60) Provisional application No. 62/819,276, filed on Mar. 15, 2019.
(60) Provisional application No. 62/673,020, filed on May 17, 2018.
(60) Provisional application No. 63/340,878, filed on May 11, 2022.

(51) Int. Cl. A61B 8/00; A61B 8/08; A61N 7/00

(52) U.S. Cl. CPC A61B 8/4427; A61B 8/0808; A61B 8/4488; A61B 8/4477; A61B 8/54; A61B 8/469; A61B 8/463; A61N 7/00; A61N 2007/0026

(57) ABSTRACT

Exemplary embodiments provide systems and methods for portable medical ultrasound for transcranial ultrasound imaging. Preferred embodiments utilize a hand portable, battery powered system having a display and a user interface operative to control imaging and display operations. A keyboard control panel can be used alone or in combination with touchscreen controls to actuate a graphical user interface. The system includes a transducer assembly to image, measure and monitor a condition of a patient or deliver a therapeutic signal to the patient.

### CROSS REFERENCE TO RELATED APPLICATIONS

This application claims priority to U.S. Provisional Application 63/340,878 filed May 11, 2022 and is also a continuation-in-part of U.S. application Ser. No. 18/090,316 filed Dec. 28, 2022 which claims priority to U.S. Provisional Patent Application No. 63/294,307, filed Dec. 28, 2021. This application is also a continuation-in-part of U.S. patent application Ser. No. 16/938,515 filed on Jul. 24, 2020, which claims priority to U.S. Provisional Application 62/878,163, filed on Jul. 24, 2019. U.S. patent application Ser. No. 16/938,515 is also is a continuation-in-part of U.S. patent application Ser. No. 16/414,215, filed May 16, 2019, which claims priority to U.S. Provisional Application No. 62/819,276 filed on Mar. 15, 2019, claims priority to U.S. Provisional Application No. 62/830,200 filed on Apr. 5, 2019, and claims priority to U.S. Provisional Application No. 62/673,020 filed on May 17, 2018.

### BACKGROUND OF THE INVENTION

Medical ultrasound imaging has become an industry standard for many medical imaging applications. In recent years, there has been an increasing need for medical ultrasound imaging equipment that is portable to allow medical personnel to easily transport the equipment to and from hospital and/or field locations, and more user-friendly to accommodate medical personnel who may possess a range of skill levels.

Conventional medical ultrasound imaging equipment typically includes at least one ultrasound probe/transducer, a keyboard and/or a knob, a computer, and a display. In a typical mode of operation, the ultrasound probe/transducer generates ultrasound waves that can penetrate tissue to different depths based on frequency level, and receives ultrasound waves reflected back from the tissue. Further, medical personnel can enter system inputs to the computer via the keyboard and/or the knob, and view ultrasound images of tissue structures on the display.

However, conventional medical ultrasound imaging equipment that employ such keyboards and/or knobs can be bulky, and therefore may not be amenable to portable use in hospital and/or field locations. Moreover, because such keyboards and/or knobs typically have uneven surfaces, they can be difficult to keep clean in hospital and/or field environments, where maintenance of a sterile field can be crucial to patient health. Some conventional medical ultrasound imaging equipment have incorporated touch screen technology to provide a partial user input interface. However, conventional medical ultrasound imaging equipment that employ such touch screen technology generally provide only limited touch screen functionality in conjunction with a traditional keyboard and/or knob, and can therefore not only be difficult to keep clean, but also complicated to use.

### SUMMARY OF THE INVENTION

In accordance with the present application, systems and methods of medical ultrasound imaging are disclosed. The presently disclosed systems and methods of medical ultrasound imaging employ medical ultrasound imaging equipment that includes a handheld housing having a laptop or a tablet form factor. The user interface can include a keyboard control panel or a multi-touch touchscreen. The system can include a graphical processing unit within the system housing that is connected to the central processor that operates to perform ultrasound imaging operations. A preferred embodiment can employ a plurality of machine learning applications including, for example, neural network for processing ultrasound image data and quantitative data generated by the system. The touchscreen interface is configured to enable selection of one or more machine learning applications from a touch actuated menu on the display. The system can utilize a shared memory within the tablet housing to access data and software modules operating on one or more processors in the tablet housing to perform one or more ultrasound imaging or data processing operations as described herein. This enables operation of third party applications running on the tablet or portable ultrasound device. A further embodiment can process image data from a second imaging modality such as a camera or other medical imaging system wherein the system processes the multimodal image data to provide overlaid images of a region of interest, for example.

Preferred embodiments can include systems and methods for automatically controlling beam transmission direction by a 2D array or by a biplane transducer array as described herein. This enables monitoring of patient organs or tissue regions in which orthogonal views can be continuously or periodically obtained. Preferred embodiments can be employed for simultaneous acquisition of parasternal short axis and long axis views of the heart, for example.

A further touchscreen enabled operation can include harmonic imaging for different imaging applications. Quantitative methods can utilize the graphics processor or core processor to apply quantitative analysis on ultrasound data including harmonic components.

Touchscreen embodiments can recognize and distinguish one or more single, multiple, and/or simultaneous touches on a surface of the touch screen display, thereby allowing the use of gestures, ranging from simple single point gestures to complex multipoint moving gestures, as user inputs to the medical ultrasound imaging equipment.

Devices and methods for ultrasound monitoring of a condition of a patient are described herein. Methods employing longer duration monitoring do not require a sonographer to remain with the patient, but they can remotely access real time acquisition of ultrasound imagery and data during the monitoring process. The user can utilize preset or selectable thresholds to set alarms to alert care providers as to a change in the condition of the patient requiring attention. The monitoring system can include a transducer assembly that can be coupled to the skin of the patient, a wound dressing or wound therapy device, so as to direct ultrasound energy into a region of interest such as an organ that requires monitoring of blood flow or other dynamic physiological process within the body. The orientation of the transducer relative to the region of interest often requires precise positioning by the user along a specific axis to insure that diagnostically useful information is being acquired continuously over the monitoring period which can extend for hours or days depending on the condition of the patient. The steering of the beam transmission axis of the transducer can be done manually, or by a mechanical or electromechanical device operated by the user. Alternatively, beam transmission axis control can be automated to maintain a preferred orientation relative to a specific region of interest or target during all or a portion of the monitoring period. The detected ultrasound signal(s) can also be monitored to maintain a certain characteristic threshold value to provide feedback control of the orientation of the beam transmission axis. A machine learning module can also be utilized to collect data regarding optimal beam direction that is used to control orientation. Embodiments can further include a therapeutic application of ultrasound energy where the axis for beam transmission of a therapeutic dose can be controlled over time. The system can control both delivery of therapeutic ultrasound energy and diagnostic measurements or imaging during a monitoring period. The system can also steer the transmission beam to track a target such as a probe, catheter or needle positioning for precise placement at a location within the region of interest. The system can preferably utilize touch actuated control on a touchscreen display to manipulate the orientation of the beam as described herein. The system can be used to monitor one or more conditions of the heart of a patient, or the flow or the accumulation of fluids at various locations in the body which are frequently symptomatic of an acute condition. The transducer probe can thus be configured as a wearable probe that is attached to the body by a transducer coupling device to render a two chamber view of the heart or a four chamber view of the heart or other portions of the vascular system including the brain where blood flow has critical functions. A first view of the heart can comprise an apical view and a second view can comprise a parasternal view of the heart and this can also be employed where two orthogonal views of other anatomical features such as the brain can be diagnostically useful. A two dimensional transducer array can be used for this purpose. Alternatively, a biplane probe as described herein can be used for visualization of different views of the heart. In a further application, the methods and devices described herein can be used to deliver a therapeutic dose of energy through the cranium into the brain of a patient during a therapy period, and/or to treat a tumor or other condition where movement of the patient does not alter the precise delivery of the ultrasound energy to a specific target point or region. The system can be used to perform a controlled scan of a region of interest in conjunction with this therapy.

In accordance with one aspect, exemplary medical ultrasound imaging system includes a housing having a front panel and a rear panel rigidly mounted to each other in parallel planes, a touch screen display, a computer having at least one processor and at least one memory, an ultrasound beamforming system, and a battery. The housing of the medical ultrasound imaging equipment is implemented in a tablet form factor. The touch screen display is disposed on the front panel of the housing, and includes a multi-touch LCD touch screen that can recognize and distinguish one or more single, multiple, and/or simultaneous touches or gestures on a surface of the touch screen display. The computer, the ultrasound beamforming system or engine, and the battery are operatively disposed within the housing. The medical ultrasound imaging equipment can use a Firewire or USB connection operatively connected between the computer and the ultrasound engine within the housing and a probe connector having a probe attach/detach lever to facilitate the connection of at least one ultrasound probe/transducer. In addition, the exemplary medical ultrasound imaging system includes an I/O port connector and a DC power input.

In an exemplary mode of operation, medical personnel can employ simple single point gestures and/or more complex multipoint gestures as user inputs to the multi-touch LCD touch screen for controlling operational modes and/or functions of the exemplary medical ultrasound imaging equipment. Such single point/multipoint gestures can correspond to single and/or multipoint touch events that are mapped to one or more predetermined operations that can be performed by the computer and/or the ultrasound engine. Medical personnel can make such single point/multipoint gestures by various finger, palm, and/or stylus motions on the surface of the touch screen display. The multi-touch LCD touch screen receives the single point/multipoint gestures as user inputs, and provides the user inputs to the computer, which executes, using the processor, program instructions stored in the memory to carry out the predetermined operations associated with the single point/multipoint gestures, at least at some times, in conjunction with the ultrasound engine. Such single point/multipoint gestures on the surface of the touch screen display can include, but are not limited to, a tap gesture, a pinch gesture, a flick gesture, a rotate gesture, a double tap gesture, a spread gesture, a drag gesture, a press gesture, a press and drag gesture, and a palm gesture. In contrast to existing ultrasound systems that rely on numerous control features operated by mechanical switching, keyboard elements, or touchpad trackball interface, preferred embodiments of the present invention employ a single on/off switch. All other operations have been implemented using touchscreen controls. Moreover, the preferred embodiments employ a capacitive touchscreen display that is sufficiently sensitive to detect touch gestures actuated by bare fingers of the user as well as gloved fingers of the user. Often medical personnel must wear sterilized plastic gloves during medical procedures. Consequently, it is highly desirable to provide a portable ultrasound device that can be used by gloved hands; however, this has previously prevented the use of touchscreen display control functions in ultrasound systems for many applications requiring sterile precautions. Preferred embodiments of the present invention provide control of all ultrasound imaging operations by gloved personnel on the touchscreen display using the programmed touch gestures.

In accordance with an exemplary aspect, at least one flick gesture may be employed to control the depth of tissue penetration of ultrasound waves generated by the ultrasound probe/transducer. For example, a single flick gesture in the “up” direction on the touch screen display surface can increase the penetration depth by one (1) centimeter or any other suitable amount, and a single flick gesture in the “down” direction on the touch screen display surface can decrease the penetration depth by one (1) centimeter or any other suitable amount. Further, a drag gesture in the “up” or “down” direction on the touch screen display surface can increase or decrease the penetration depth in multiples of one (1) centimeter or any other suitable amount. Additional operational modes and/or functions controlled by specific single point/multipoint gestures on the touch screen display surface can include, but are not limited to, freeze/store operations, 2-dimensional mode operations, gain control, color control, split screen control, PW imaging control, cine/time-series image clip scrolling control, zoom and pan control, full screen control, Doppler and 2-dimensional beam steering control, and/or body marking control. At least some of the operational modes and/or functions of the exemplary medical ultrasound imaging equipment can be controlled by one or more touch controls implemented on the touch screen display in which beamforming parameters can be reset by moving touch gestures. Medical personnel can provide one or more specific single point/multipoint gestures as user inputs for specifying at least one selected subset of the touch controls to be implemented, as required and/or desired, on the touch screen display. A larger number of touchscreen controls enable greater functionality when operating in full screen mode when a few or more virtual buttons or icons are available for use.

In accordance with another exemplary aspect, a press gesture can be employed inside a region of the touch screen display, and, in response to the press gesture, a virtual window can be provided on the touch screen display for displaying at least a magnified portion of an ultrasound image displayed on the touch screen display. In accordance with still another exemplary aspect, a press and drag gesture can be employed inside the region of the touch screen display, and, in response to the press and drag gesture, a predetermined feature of the ultrasound image can be traced. Further, a tap gesture can be employed inside the region of the touch screen display, substantially simultaneously with a portion of the press and drag gesture, and, in response to the tap gesture, the tracing of the predetermined feature of the ultrasound image can be completed. These operations can operate in different regions of a single display format, so that a moving gesture within a region of interest within the image, for example, may perform a different function than the same gesture executed within the image but outside the region of interest.

By providing medical ultrasound imaging equipment with a multi-touch touchscreen, medical personnel can control the equipment using simple single point gestures and/or more complex multipoint gestures, without the need of a traditional keyboard or knob. Because the multi-touch touch screen obviates the need for a traditional keyboard or knob, such medical ultrasound imaging equipment is easier to keep clean in hospital and/or field environments, provides an intuitive user friendly interface, while providing fully functional operations. Moreover, by providing such medical ultrasound imaging equipment in a tablet form factor, medical personnel can easily transport the equipment between hospital and/or field locations.

Certain exemplary embodiments provide a multi-chip module for an ultrasound engine of a portable medical ultrasound imaging system, in which a transmit/receive (TR) chip, a pre-amp/time gain compensation (TGC) chip and a beamformer chip are assembled in a vertically stacked configuration. The transmission circuit provides high voltage electrical driving pulses to the transducer elements to generate a transmit beam. As the transmit chip operates at voltages greater than 80V, a CMOS process utilizing a 1 micron design rule has been utilized for the transmit chip and a submicron design rule has been utilized for the low-voltage receiving circuits (less than 5V).

Preferred embodiments of the present invention utilize a submicron process to provide integrated circuits with sub-circuits operating at a plurality of voltages, for example, 2.5V, 5V and 60V or higher. These features can be used in conjunction with a bi-plane transducer probe in accordance with certain preferred embodiments of the invention.

Thus, a single IC chip can be utilized that incorporates high voltage transmission, low voltage amplifier/TGC and low voltage beamforming circuits in a single chip. Using a 0.25 micron design rule, this mixed signal circuit can accommodate beamforming of 32 transducer channels in a chip area less than 0.7×0.7 (0.49) cm². Thus, 128 channels can be processed using four 32 channel chips in a total circuit board area of less than 1.5×1.5 (2.25) cm².

The term “multi-chip module,” as used herein, refers to an electronic package in which multiple integrated circuits (IC) are packaged with a unifying substrate, facilitating their use as a single component, i.e., as a higher processing capacity IC packaged in a much smaller volume. Each IC can comprise a circuit fabricated in a thinned semiconductor wafer. Exemplary embodiments also provide an ultrasound engine including one or more such multi-chip modules, and a portable medical ultrasound imaging system including an ultrasound engine circuit board with one or more multi-chip modules. Exemplary embodiments also provide methods for fabricating and assembling multi-chip modules as taught herein. Vertically stacking the TR chip, the pre-amp/TGC chip, and the beamformer chip on a circuit board minimizes the packaging size (e.g., the length and width) and the footprint occupied by the chips on the circuit board.

The TR chip, the pre-amp/TGC chip, and the beamformer chip in a multi-chip module may each include multiple channels (for example, 8 channels per chip to 64 channels per chip). In certain embodiments, the high-voltage TR chip, the pre-amp/TGC chip, and the sample-interpolate receive beamformer chip may each include 8, 16, 32, 64 channels. In a preferred embodiment, each circuit in a two layer beamformer module has 32 beamformer receive channels to provide a 64 channel receiving beamformer. A second 64 channel two layer module can be used to form a 128 channel handheld tablet ultrasound device having an overall thickness of less than 2 cm. A transmit multi-chip beamformer can also be used having the same or similar channel density in each layer.

Exemplary numbers of chips vertically integrated in a multi-chip module may include, but are not limited to, two, three, four, five, six, seven, eight, and the like. In one embodiment of an ultrasound device, a single multi-chip module is provided on a circuit board of an ultrasound engine that performs ultrasound-specific operations. In other embodiments, a plurality of multi-chip modules are provided on a circuit board of an ultrasound engine. The plurality of multi-chip modules may be stacked vertically on top of one another on the circuit board of the ultrasound engine to further minimize the packaging size and the footprint of the circuit board.

Providing one or more multi-chip modules on a circuit board of an ultrasound engine achieves a high channel count while minimizing the overall packaging size and footprint. For example, a 128-channel ultrasound engine circuit board can be assembled, using multi-chip modules, within exemplary planar dimensions of about 10 cm×about 10 cm, which is a significant improvement over the much larger space requirements of conventional ultrasound circuits. A single circuit board of an ultrasound engine including one or more multi-chip modules may have 16 to 128 channels in some embodiments. In certain embodiments, a single circuit board of an ultrasound engine including one or more multi-chip modules may have 16, 32, 64, 128 or 192 channels, and the like.

Preferred embodiments of tablet ultrasound systems utilize a graphics processor configured to perform machine learning operations using the acquired images to perform automated image processing and guidance for real time imaging procedures. Such machine learning operations can be performed on both the main system processor and the graphics processor in which automated computational techniques utilize iterative processes in which a selected metric converges to a stored reference level or rating to define a set of images or computed values used for diagnosis.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings, in which:

FIG. 1A is a plan view of exemplary medical ultrasound imaging equipment, in accordance with an exemplary embodiment of the present application;

FIG. 1B shows a battery powered portable system having a keyboard control panel and a folding display;

FIGS. 2A and 2B are side views of the medical ultrasound imaging system in accordance with preferred embodiments of the invention;

FIGS. 3AA 3AL illustrates exemplary single point and multipoint gestures that can be employed as user inputs to the medical ultrasound imaging system in accordance with preferred embodiments of the invention;

FIG. 3B illustrates a process flow diagram for operating a tablet ultrasound system in accordance with preferred embodiments of the invention;

FIG. 3C-3K illustrates details of touchscreen gestures to adjust beamforming and display operation;

FIGS. 4A-4C illustrates exemplary subsets of touch controls that can be implemented on the medical ultrasound imaging system in accordance with preferred embodiments of the invention;

FIGS. 5A and 5B are exemplary representations of a liver with a cystic lesion on a touch screen display of the medical ultrasound imaging system in accordance with preferred embodiments of the invention;

FIGS. 5C and 5D are exemplary representations of the liver and cystic lesion on the touch screen display of FIGS. 5A and 5B, including a virtual window that corresponds to a magnified portion of the liver;

FIG. 6A is an exemplary representation of an apical four (4) chamber view of a heart on the touch screen display of the medical ultrasound imaging system;

FIGS. 6B-6E illustrates an exemplary manual tracing of an endocardial border of a left ventricle of the heart on the touch screen display of FIG. 6A;

FIGS. 7A-7C illustrates an exemplary measurement of the size of the cystic lesion on the liver within the virtual window of FIGS. 5C and 5D;

FIGS. 8A-8C illustrates an exemplary caliper measurement of the cystic lesion on the liver within the virtual window of FIGS. 5C and 5D;

FIG. 9A illustrates one of a plurality of transducer arrays attached to the processor housing;

FIG. 9B shows a transducer attach sequence in accordance with exemplary embodiments;

FIG. 10A shows a method of measuring heart wall motion;

FIG. 10B shows a schematic block diagram for an integrated ultrasound probe with exemplary embodiments;

FIG. 10C shows a schematic block diagram for an integrated ultrasound probe with exemplary embodiments;

FIG. 11 is a detailed schematic block diagram of an exemplary embodiment of an ultrasound engine (i.e., the front-end ultrasound specific circuitry) and an exemplary embodiment of a computer motherboard (i.e., the host computer) of the exemplary ultrasound device;

FIG. 12 is a detailed schematic block diagram of an exemplary embodiment of an ultrasound engine (i.e., the front-end ultrasound specific circuitry) and an exemplary embodiment of a computer motherboard (i.e., the host computer) provided as a single board complete ultrasound system;

FIG. 13 is a perspective view of an exemplary portable ultrasound system provided in accordance with exemplary embodiments;

FIG. 14 illustrates an exemplary view of a main graphical user interface (GUI) rendered on a touch screen display of the exemplary portable ultrasound system of FIG. 18;

FIGS. 15A and 15B are top views of the medical ultrasound imaging systems in accordance with another preferred embodiment of the invention;

FIG. 16 illustrates a preferred cart system for a tablet ultrasound system in accordance with preferred embodiments of the invention;

FIG. 17 illustrates preferred cart system for a modular ultrasound imaging system in accordance with preferred embodiments of the invention;

FIGS. 18A and 18B illustrating preferred cart systems for a modular ultrasound imaging system in accordance with preferred embodiments of the invention;

FIG. 19 illustrates a 2D imaging mode of operation with a modular ultrasound imaging system in accordance with the invention;

FIG. 20 illustrates a motion mode of operation with a modular ultrasound imaging system in accordance with the invention;

FIG. 21 illustrates a color Doppler mode of operation with a modular ultrasound imaging system in accordance with the invention;

FIG. 22 illustrates a pulsed-wave Doppler mode of operation with a modular ultrasound imaging system in accordance with the invention;

FIG. 23 illustrates a Triplex scan mode of operation with a modular ultrasound imaging system in accordance with the invention;

FIG. 24 illustrates a GUI Home Screen interface for a user mode of operation with a modular ultrasound imaging system in accordance with the invention;

FIG. 25 illustrates a GUI Menu Screen Interface for a user mode of operation with a modular ultrasound imaging system in accordance with the invention;

FIG. 26 illustrates a GUI Patient Data Screen Interface for a user mode of operation with a modular ultrasound imaging system in accordance with the invention;

FIG. 27 illustrates a GUI Pre-sets Screen Interface for a user mode of operation with a modular ultrasound imaging system in accordance with the invention;

FIG. 28 illustrates a GUI Review Screen Interface for a user mode of operation with a modular ultrasound imaging system in accordance with the invention;

FIGS. 29A-29C illustrate XY bi-plane probe comprising two one-dimensional (1D) multi-element arrays in accordance with a preferred embodiment of the invention;

FIG. 30 illustrates the operation of a bi-plane image forming xy-probe;

FIG. 31 illustrates the operation of a bi-plane image forming xy-probe;

FIG. 32 illustrates a high voltage driver circuit for a bi-plane image forming xy-probe;

FIGS. 33A-33B illustrate simultaneous bi-plane evaluation of left ventricular condition;

and

FIGS. 34A and 34B illustrate ejection fraction probe measurement techniques in accordance with preferred embodiments of the invention;

FIG. 35 shows the calculated acoustic pressure level at the fundamental frequency, 2nd harmonics frequency and superharmonic frequency in tissue at the focal distance as a function of lateral distance in mm;

FIG. 36 shows the fundamental, 2nd and 3 rd harmonic beam profile;

FIG. 37 illustrates an imaging sequence using position tracking of a transducer probe.

FIG. 38A illustrates a computational neural network model with fully connected artificial neural nodes in accordance with various embodiments of the present application.

FIG. 38B illustrates a portion of a radial basis function classifier model with input and hidden layers in accordance with various embodiments of the present application.

FIG. 39A illustrates an exemplary artificial intelligence application data flow in accordance with various embodiments described herein.

FIG. 39B depicts a photograph of a circuit board layout for a tablet configuration in accordance with various embodiments.

FIG. 40 illustrates the use of a shared memory to provide communication with an external application in accordance with various embodiments described herein.

FIG. 41 illustrates a distributed processing system.

FIG. 42 illustrates a keyboard control panel for a portable ultrasound system.

FIG. 43 illustrates a plurality of softkeys displayed on the imaging window.

FIG. 44 illustrates a cross-sectional view of a tablet ultrasound device according to various embodiments.

FIG. 45 illustrates a bottom schematic view of the tablet ultrasound device in accordance with various embodiments described herein with the bottom portion of the housing and the ultrasound engine removed.

FIG. 46 illustrates a schematic view of the display of the tablet ultrasound device in accordance with various embodiments described herein.

FIG. 47 illustrates a preferred embodiment of a wearable XY-probe.

FIG. 48 illustrates a preferred embodiment of an XY-probe acoustic module with magnetic actuators.

FIGS. 49A and 49B illustrate a linear motor with actuator in different states.

FIGS. 50A-50C show three actuators packaged together in perspective, top and side views, respectively.

FIG. 51 illustrates three actuators with a flat top plate as the reference for tilting.

FIG. 52 illustrates an actuator and acoustic module assembly.

FIG. 53 illustrates an example of programming the three motors to have different extended lengths along the z-axis.

FIG. 54 is an example of programming the three motors to have different extended lengths along the z-axis resulting in a tilt in the XY probe acoustic module due to the different lengths of the three actuators.

FIG. 55 illustrates a cardiac output measurement in apical view FIG. 56 illustrates the cardiac output measurement in parasternal view.

FIG. 57 depicts a tilt control feature as a user interface on a touch screen control.

FIG. 58 depicts a 4C apical view with LV measurement based on the Simpson method at the end of Diastole.

FIG. 59 depicts a 2C apical view with LV measurement based on the Simpson method at the end of Diastole.

FIG. 60 depicts left ventricle volume calculation only using LV diameter (D).

FIG. 61 depicts a parasternal view measured at the end of diastole.

FIG. 62 depicts a parasternal view measured at the end of systole.

FIG. 63 depicts a 2D-image in anatomic M-Mode provided in an exemplary embodiment.

FIG. 64 depicts a 2D Plax view allowing the proper placement of the anatomic m-mode.

FIGS. 65A-65B depict UI feedback for probe manipulations provided in exemplary embodiments.

FIG. 66 depicts exemplary UI feedback presenting swing (asymmetry) in an exemplary embodiment.

FIG. 67 depicts exemplary LV rings.

FIG. 68 depicts exemplary probe orientations.

FIG. 69 depicts the y-probe in correct position in an exemplary embodiment.

FIG. 70 illustrates an ultrasound system performing a transcranial imaging operation in accordance with some embodiments taught herein.

FIG. 71 illustrates a flowchart for a method of transcranial imaging or therapy in accordance with embodiments taught herein.

FIG. 72A illustrates a treatment regimen for experimental and control groups for a course of treatment using low-intensity pulsed ultrasound (LIPUS) in accordance with some embodiments taught herein.

FIG. 72B illustrates a transcranial ultrasound device as applied to a mouse cranium in accordance with some embodiments taught herein.

FIG. 73 schematically illustrates an ultrasound tablet or laptop device having first and second byplane tranducers.

FIG. 74 schematically illustrates a high voltage switching device to simultaneously control imaging with first and second byplane transducers.

### DETAILED DESCRIPTION

Systems and methods of medical ultrasound imaging are disclosed. The presently disclosed systems and methods of medical ultrasound imaging employ medical ultrasound imaging equipment that includes housing in a tablet form factor, and a touch screen display disposed on a front panel of the housing. The touch screen display includes a multi-touch touch screen that can recognize and distinguish one or more single, multiple, and/or simultaneous touches on a surface of the touch screen display, thereby allowing the use of gestures, ranging from simple single point gestures to complex multipoint gestures, as user inputs to the medical ultrasound imaging equipment. Further details regarding tablet ultrasound systems and operations are described in U.S. application Ser. No. 10/997,062 filed on Nov. 11, 2004, Ser. No. 10/386,360 filed Mar. 11, 2003 and U.S. Pat. No. 6,969,352, the entire contents of these patents and applications are incorporated herein by reference.

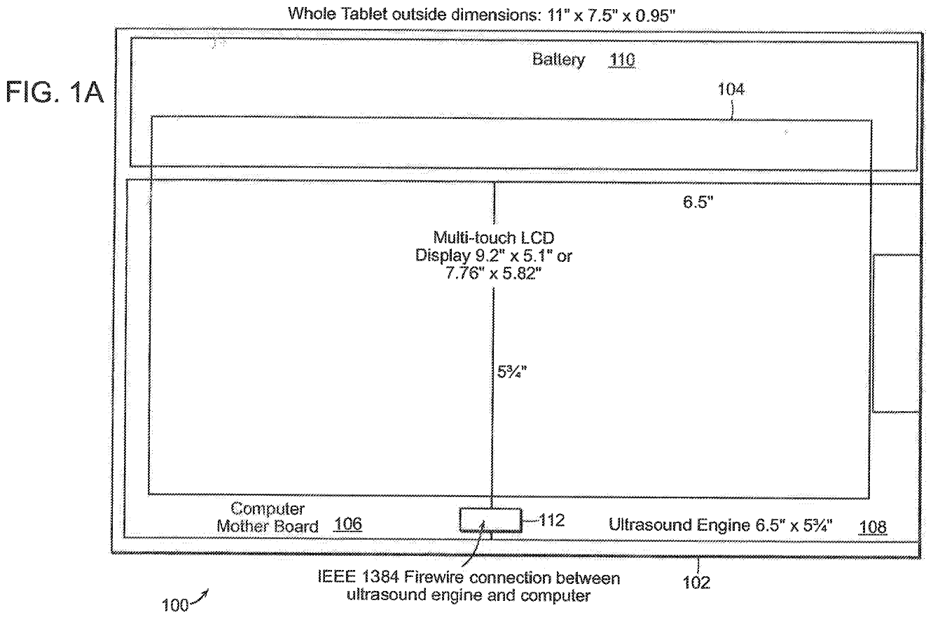

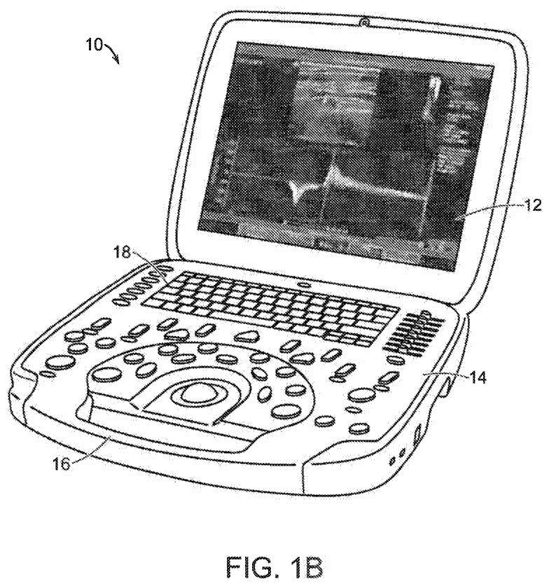

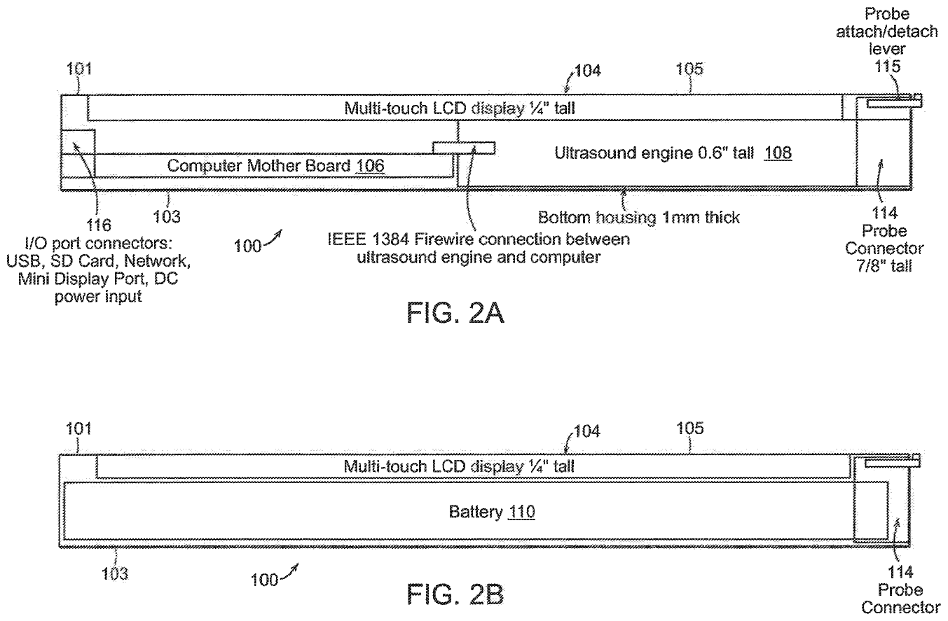

FIGS. 1A and 1B depict illustrative embodiments of exemplary medical ultrasound imaging equipment 10, 100, in accordance with the present application. As shown in FIG. 1A, the medical ultrasound imaging equipment 100 includes a housing 102, a touch screen display 104, a computer having at least one processor and at least one memory implemented on a computer motherboard 106, an ultrasound engine 108, and a battery 110. For example, the housing 102 can be implemented in a tablet form factor, or any other suitable form factor. The housing 102 has a front panel 101 and a rear panel 103. The touch screen display 104 is disposed on the front panel 101 of the housing 102, and includes a multi-touch LCD touch screen that can recognize and distinguish one or more multiple and/or simultaneous touches on a surface 105 of the touch screen display 104. The computer motherboard 106, the ultrasound engine 108, and the battery 110 are operatively disposed within the housing 102. The medical ultrasound imaging equipment 100 further includes a Firewire connection 112 (see also FIG. 2A) operatively connected between the computer motherboard 106 and the ultrasound engine 108 within the housing 102, and a probe connector 114 having a probe attach/detach lever 115 (see also FIGS. 2A and 2B) to facilitate the connection of at least one ultrasound probe/transducer. The transducer probe housing can include circuit components including a transducer array, transmit and receive circuitry, as well as beamformer and beamformer control circuits in certain preferred embodiments. In addition, the medical ultrasound imaging equipment 100 has one or more I/O port connectors 116 (see FIG. 2A), which can include, but are not limited to, one or more USB connectors, one or more SD cards, one or more network ports, one or more mini display ports, and a DC power input. A further embodiment shown in FIG. 1B employs a battery powered hand portable system weighing less than 15 lbs. that has a folding display 12 and a keyboard control panel 14 having a keyboard 18 and a handle 16.

In an exemplary mode of operation, medical personnel (also referred to herein as the “user” or “users”) can employ simple single point gestures and/or more complex multipoint gestures as user inputs to the multi-touch LCD touch screen of the touch screen display 104 for controlling one or more operational modes and/or functions of the medical ultrasound imaging equipment 100. Such a gesture is defined herein as a movement, a stroke, or a position of at least one finger, a stylus, and/or a palm on the surface 105 of the touch screen display 104. For example, such single point/multipoint gestures can include static or dynamic gestures, continuous or segmented gestures, and/or any other suitable gestures. A single point gesture is defined herein as a gesture that can be performed with a single touch contact point on the touch screen display 104 by a single finger, a stylus, or a palm. A multipoint gesture is defined herein as a gesture that can be performed with multiple touch contact points on the touch screen display 104 by multiple fingers, or any suitable combination of at least one finger, a stylus, and a palm. A static gesture is defined herein as a gesture that does not involve the movement of at least one finger, a stylus, or a palm on the surface 105 of the touch screen display 104. A dynamic gesture is defined herein as a gesture that involves the movement of at least one finger, a stylus, or a palm, such as the movement caused by dragging one or more fingers across the surface 105 of the touch screen display 104. A continuous gesture is defined herein as a gesture that can be performed in a single movement or stroke of at least one finger, a stylus, or a palm on the surface 105 of the touch screen display 104. A segmented gesture is defined herein as a gesture that can be performed in multiple movements or strokes of at least one finger, a stylus, or a palm on the surface 105 of the touch screen display 104.

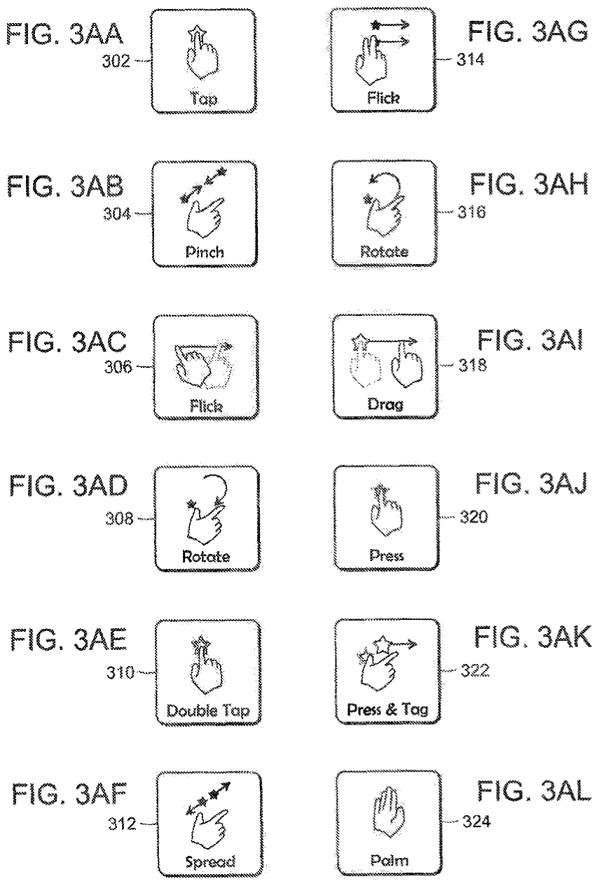

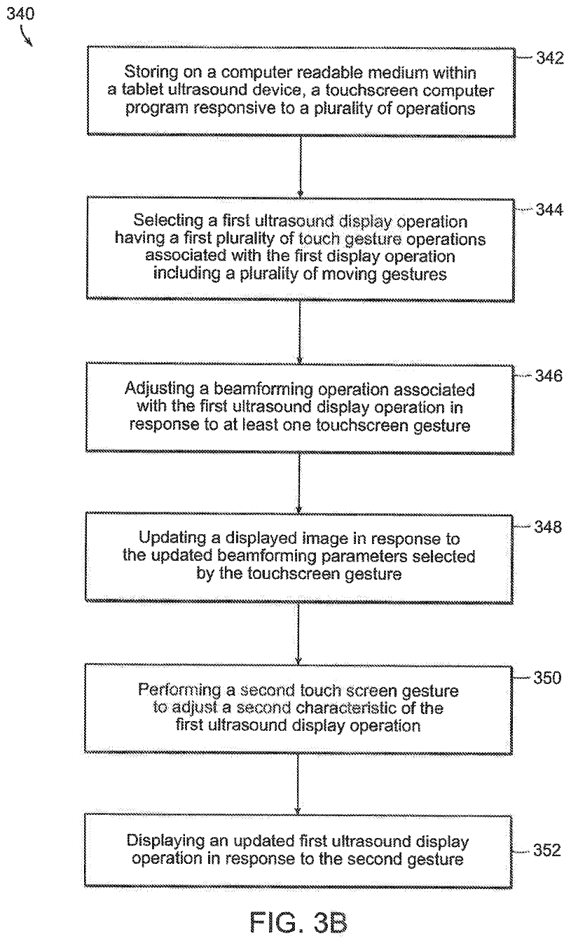

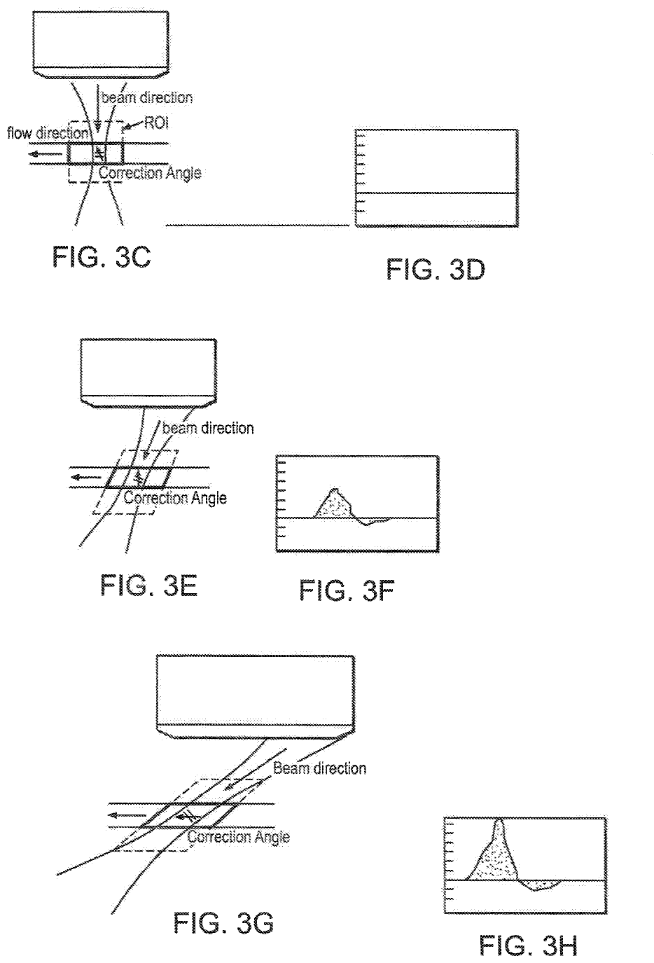

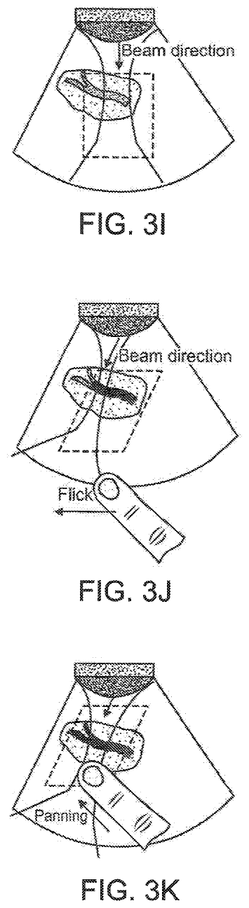

Such single point/multipoint gestures performed on the surface 105 of the touch screen display 104 can correspond to single or multipoint touch events, which are mapped to one or more predetermined operations that can be performed by the computer and/or the ultrasound engine 108. Users can make such single point/multipoint gestures by various single finger, multi-finger, stylus, and/or palm motions on the surface 105 of the touch screen display 104. The multi-touch LCD touch screen receives the single point/multipoint gestures as user inputs, and provides the user inputs to the processor, which executes program instructions stored in the memory to carry out the predetermined operations associated with the single point/multipoint gestures, at least at some times, in conjunction with the ultrasound engine 108. As shown in FIGS. 3AA-3AL, such single point/multipoint gestures on the surface 105 of the touch screen display 104 can include, but are not limited to, a tap gesture 302, a pinch gesture 304, a flick gesture 306, 314, a rotate gesture 308, 316, a double tap gesture 310, a spread gesture 312, a drag gesture 318, a press gesture 320, a press and drag gesture 322, and/or a palm gesture 324. For example, such single point/multipoint gestures can be stored in at least one gesture library in the memory implemented on the computer motherboard 106. The computer program operative to control system operations can be stored on a computer readable medium and can optionally be implemented using a touch processor connected to an image processor and a control processor connected to the system beamformer. Thus beamformer delays associated with both transmission and reception can be adjusted in response to both static and moving touch gestures. In accordance with the illustrative embodiment of FIG. 1A, at least one flick gesture 306 or 314 may be employed by a user of the medical ultrasound imaging equipment 100 to control the depth of tissue penetration of ultrasound waves generated by the ultrasound probe/transducer. For example, a dynamic, continuous, flick gesture 306 or 314 in the “up” direction, or any other suitable direction, on the surface 105 of the touch screen display 104 can increase the penetration depth by one (1) centimeter, or any other suitable amount. Further, a dynamic, continuous, flick gesture 306 or 314 in the “down” direction, or any other suitable direction, on the surface 105 of the touch screen display 104 can decrease the penetration depth by one (1) centimeter, or any other suitable amount. Moreover, a dynamic, continuous, drag gesture 318 in the “up” or “down” direction, or any other suitable direction, on the surface 105 of the touch screen display 104 can increase or decrease the penetration depth in multiple centimeters, or any other suitable amounts.

Additional operational modes and/or functions controlled by specific single point/multipoint gestures on the surface 105 of the touch screen display 104 can include, but are not limited to, freeze/store operations, 2-dimensional mode operations, gain control, color control, split screen control, PW imaging control, cine/time-series image clip scrolling control, zoom and pan control, full screen display, Doppler and 2-dimensional beam steering control, and/or body marking control. At least some of the operational modes and/or functions of the medical ultrasound imaging equipment 100 can be controlled by one or more touch controls implemented on the touch screen display 104. Further, users can provide one or more specific single point/multipoint gestures as user inputs for specifying at least one selected subset of the touch controls to be implemented, as required and/or desired, on the touch screen display 104.

Shown in FIG. 3B is a process sequence in which ultrasound beamforming and imaging operations 340 are controlled in response to touch gestures entered on a touchscreen. Various static and moving touch gestures have been programmed into the system such that the data processor operable to control beamforming and image processing operations 342 within the tablet device. A user can select 344 a first display operation having a first plurality of touch gestures associated therewith. Using a static or moving gesture the user can perform one of the plurality of gestures operable to control the imaging operation and can specifically select one of a plurality of gestures that can adjust beamforming parameters 346 being used to generate image data associated with the first display operation. The displayed image is updated and displayed 348 response to the updated beamforming procedure. The user can further elect to perform a different gesture having a different velocity characteristic (direction or speed or both) to adjust 350 a second characteristic of the first ultrasound display operation. The displayed image is then updated 352 based on the second gesture, which can modify imaging processing parameters or beamforming parameters. Examples of this process are described in further detail herein where changes in velocity and direction of different gestures can be associated with distinct imaging parameters of a selected display operation.

Ultrasound images of flow or tissue movement, whether color flow or spectral Doppler, are essentially obtained from measurements of movement. In ultrasound scanners, a series of pulses is transmitted to detect movement of blood. Echoes from stationary targets are the same from pulse to pulse. Echoes from moving scatterers exhibit slight differences in the time for the signal to be returned to the scanner.

As can be seen from FIG. 3C-3H, there has to be motion in the direction of the beam; if the flow is perpendicular to the beam, there is no relative motion from pulse to pulse receive, there is no flow detected. These differences can be measured as a direct time difference or, more usually, in terms of a phase shift from which the ‘Doppler frequency’ is obtained. They are then processed to produce either a color flow display or a Doppler sonogram. In FIG. 3C-3D, the flow direction is perpendicular to the beam direction, no flow is measured by Pulse Wave spectral Doppler. In FIG. 3G-3H when the ultrasound beam is steered to an angle that is better aligned to the flow, a weak flow is shown in the color flow map, and in addition flow is measured by Pulse Wave Doppler. In FIG. 3H, when the ultrasound beam is steered to an angle much better aligned to the flow direction in response to a moving, the color flow map is stronger, in addition when the correction angle of the PWD is placed aligned to the flow, a strong flow is measured by the PWD.

In this tablet ultrasound system, an ROI, region of interest, is also used to define the direction in response to a moving gesture of the ultrasound transmit beam. A liver image with a branch of renal flow in color flow mode is shown in FIG. 3I since the ROI is straight down from the transducer, the flow direction is almost normal to the ultrasound beam, so very weak renal flow is detected. Hence, the color flow mode is used to image a renal flow in liver. As can be seen, the beam is almost normal to the flow and very weak flow is detected. A flick gesture with the finger outside of the ROI is used to steer the beam. As can be seen in FIG. 3J, the ROI is steered by resetting beamforming parameters so that the beam direction is more aligned to the flow direction, a much stronger flow within the ROI is detected. In FIG. 3J, a flick gesture with the finger outside of the ROI is used to steer the ultrasound beam into the direction more aligned to the flow direction. Stronger flow within the ROI can be seen. A panning gesture with the finger inside the ROI will move the ROI box into a position that covers the entire renal region, i.e., panning allows a translation movement of the ROI box such that the box covers the entire target area.

FIG. 3K demonstrates a panning gesture. With the finger inside the ROI, it can move the ROI box to any place within the image plane. In the above embodiment, it is easy to differentiate a “flick” gesture with a finger outside an “ROI” box is intended for steering a beam, and a “drag-and-move, panning” gesture with a finger inside the “ROI” is intended for moving the ROI box. However, there are applications in which no ROI as a reference region, then it is easy to see that it is difficult to differentiate a “flick” or a “panning” gesture, in this case, the touch-screen program needs to track the initial velocity or acceleration of the finger to determine it is a “flick” gesture or a “drag-and-move” gesture. Thus, the touch engine that receives data from the touchscreen sensor device is programmed to discriminate between velocity thresholds that indicate different gestures. Thus, the time, speed and direction associated with different moving gestures can have preset thresholds. Two and three finger static and moving gestures can have separate thresholds to differentiate these control operations. Note that preset displayed icons or virtual buttons can have distinct static pressure or time duration thresholds. When operated in full screen mode, the touchscreen processor, which is preferably operating on the systems central processing unit that performs other imaging operations such as scan conversion, switches off the static icons.

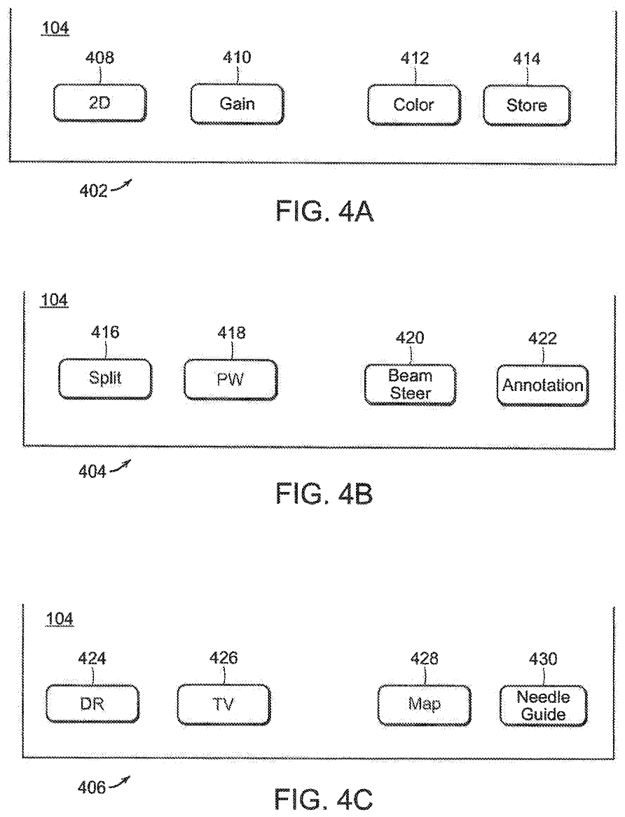

FIGS. 4A-4C depict exemplary subsets 402, 404, 406 of touch controls that can be implemented by users of the medical ultrasound imaging equipment 100 on the touch screen display 104. It is noted that any other suitable subset(s) of touch controls can be implemented, as required and/or desired, on the touch screen display 104. As shown in FIG. 4A, the subset 402 includes a touch control 408 for performing 2-dimensional (2D) mode operations, a touch control 410 for performing gain control operations, a touch control 412 for performing color control operations, and a touch control 414 for performing image/clip freeze/store operations. For example, a user can employ the press gesture 320 to actuate the touch control 408, returning the medical ultrasound imaging equipment 100 to 2D mode. Further, the user can employ the press gesture 320 against one side of the touch control 410 to decrease a gain level, and employ the press gesture 320 against another side of the touch control 410 to increase the gain level. Moreover, the user can employ the drag gesture 318 on the touch control 412 to identify ranges of densities on a 2D image, using a predetermined color code. In addition, the user can employ the press gesture 320 to actuate the touch control 414 to freeze/store a still image or to acquire a cine image clip.

As shown in FIG. 4B, the subset 404 includes a touch control 416 for performing split screen control operations, a touch control 418 for performing PW imaging control operations, a touch control 420 for performing Doppler and 2-dimensional beam steering control operations, and a touch control 422 for performing annotation operations. For example, a user can employ the press gesture 320 against the touch control 416, allowing the user to toggle between opposing sides of the split touch screen display 104 by alternately employing the tap gesture 302 on each side of the split screen. Further, the user can employ the press gesture 320 to actuate the touch control 418 and enter the PW mode, which allows (1) user control of the angle correction, (2) movement (e.g., “up” or “down”) of a baseline that can be displayed on the touch screen display 104 by employing the press and drag gesture 322, and/or (3) an increase or a decrease of scale by employing the tap gesture 302 on a scale bar that can be displayed on the touch screen display 104. Moreover, the user can employ the press gesture 320 against one side of the touch control 420 to perform 2D beam steering to the “left” or any other suitable direction in increments of five (5) or any other suitable increment, and employ the press gesture 320 against another side of the touch control 420 to perform 2D beam steering to the “right” or any other suitable direction in increments of five (5) or any other suitable increment. In addition, the user can employ the tap gesture 302 on the touch control 422, allowing the user to enter annotation information via a pop-up keyboard that can be displayed on the touch screen display 104.

As shown in FIG. 4C, the subset 406 includes a touch control 424 for performing dynamic range operations, a touch control 426 for performing Teravision™ software operations, a touch control 428 for performing map operations, and a touch control 430 for performing needle guide operations. For example, a user can employ the press gesture 320 and/or the press and drag gesture 322 against the touch control 424 to control or set the dynamic range. Further, the user can employ the tap gesture 302 on the touch control 426 to choose a desired level of the Teravision™ software to be executed from the memory by the processor on the computer motherboard 106. Moreover, the user can employ the tap gesture 302 on the touch control 428 to perform a desired map operation. In addition, the user can employ the press gesture 320 against the touch control 430 to perform a desired needle guide operation.

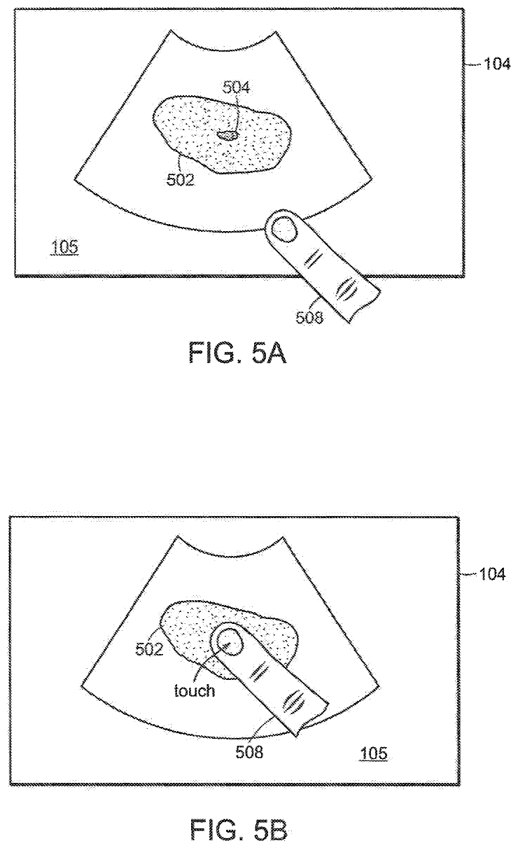

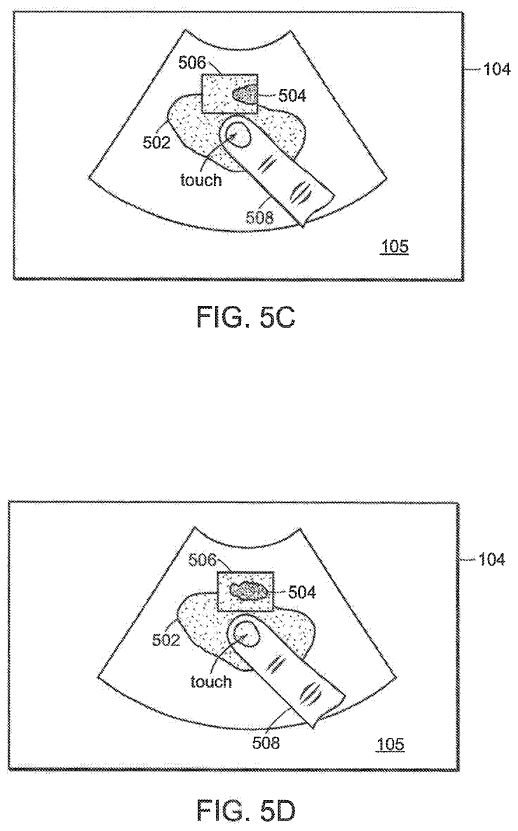

In accordance with the present application, various measurements and/or tracings of objects (such as organs, tissues, etc.) displayed as ultrasound images on the touch screen display 104 of the medical ultrasound imaging equipment 100 (see FIG. 1A) can be performed, using single point/multipoint gestures on the surface 105 of the touch screen display 104. The user can perform such measurements and/or tracings of objects directly on an original ultrasound image of the displayed object, on a magnified version of the ultrasound image of the displayed object, and/or on a magnified portion of the ultrasound image within a virtual window 506 (see FIGS. 5C and 5D) on the touch screen display 104.

FIGS. 5A and 5B depict an original ultrasound image of an exemplary object, namely, a liver 502 with a cystic lesion 504, displayed on the touch screen display 104 of the medical ultrasound imaging equipment 100 (see FIG. 1). It is noted that such an ultrasound image can be generated by the medical ultrasound imaging equipment 100 in response to penetration of the liver tissue by ultrasound waves generated by an ultrasound probe/transducer operatively connected to the equipment 100. Measurements and/or tracings of the liver 502 with the cystic lesion 504 can be performed directly on the original ultrasound image displayed on the touch screen display 104 (see FIGS. 5A and 5B), or on a magnified version of the ultrasound image. For example, the user can obtain such a magnified version of the ultrasound image using a spread gesture (see, e.g., the spread gesture 312; FIG. 3) by placing two (2) fingers on the surface 105 of the touch screen display 104, and spreading them apart to magnify the original ultrasound image. Such measurements and/or tracings of the liver 502 and cystic lesion 504 can also be performed on a magnified portion of the ultrasound image within the virtual window 506 (see FIGS. 5C and 5D) on the touch screen display 104.

For example, using his or her finger (see, e.g., a finger 508; FIGS. 5A-5D), the user can obtain the virtual window 506 by employing a press gesture (see, e.g., the press gesture 320; FIG. 3) against the surface 105 of the touch screen display 104 (see FIG. 5B) in the vicinity of a region of interest, such as the region corresponding to the cystic lesion 504. In response to the press gesture, the virtual window 506 (see FIGS. 5C and 5D) is displayed on the touch screen display 104, possibly at least partially superimposed on the original ultrasound image, thereby providing the user with a view of a magnified portion of the liver 502 in the vicinity of the cystic lesion 504. For example, the virtual window 506 of FIG. 5C can provide a view of a magnified portion of the ultrasound image of the cystic lesion 504, which is covered by the finger 508 pressed against the surface 105 of the touch screen display 104. To re-position the magnified cystic lesion 504 within the virtual window 506, the user can employ a press and drag gesture (see, e.g., the press and drag gesture 322; FIG. 3) against the surface 105 of the touch screen display 104 (see FIG. 5D), thereby moving the image of the cystic lesion 504 to a desired position within the virtual window 506. In one embodiment, the medical ultrasound imaging equipment 100 can be configured to allow the user to select a level of magnification within the virtual window 506 to be 2 times larger, 4 times larger, or any other suitable number of times larger than the original ultrasound image. The user can remove the virtual window 506 from the touch screen display 104 by lifting his or her finger (see, e.g., the finger 508; FIGS. 5A-5D) from the surface 105 of the touch screen display 104.

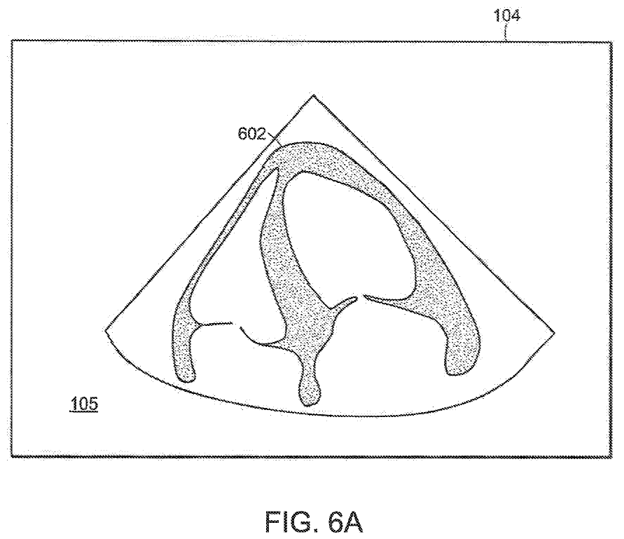

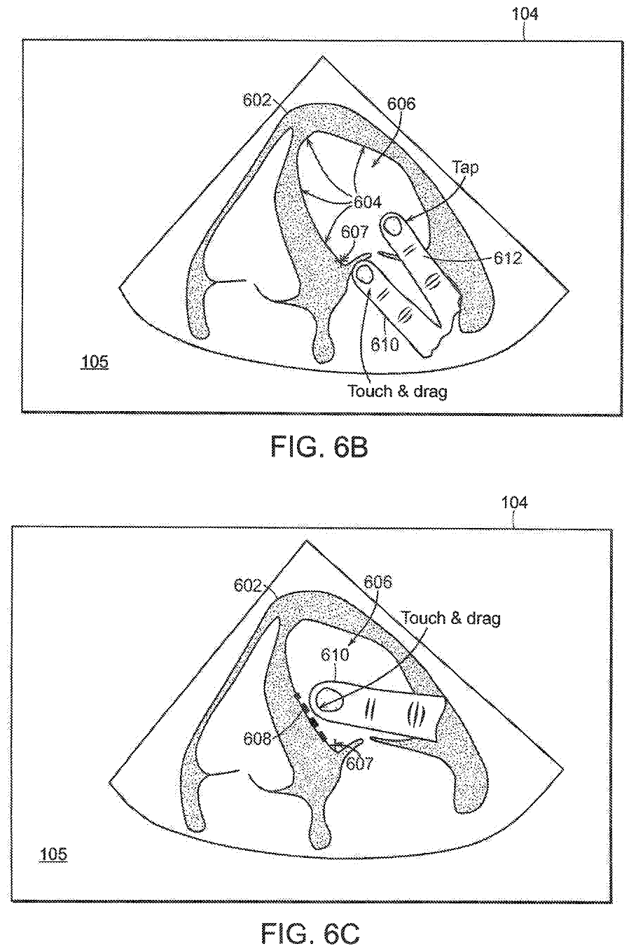

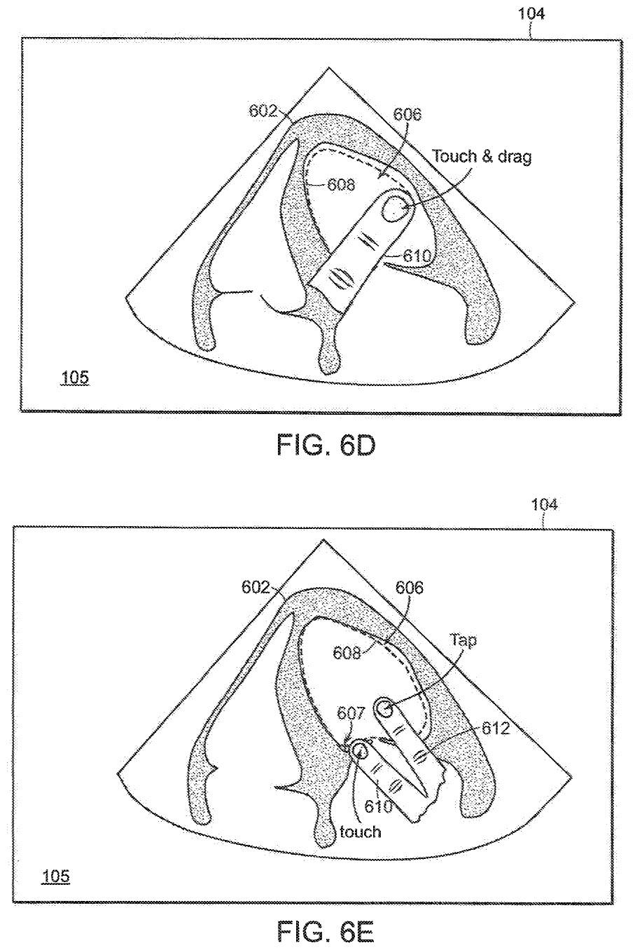

FIG. 6A depicts an ultrasound image of another exemplary object, namely, an apical four (4) chamber view of a heart 602, displayed on the touch screen display 104 of the medical ultrasound imaging equipment 100 (see FIG. 1). It is noted that such an ultrasound image can be generated by the medical ultrasound imaging equipment 100 in response to penetration of the heart tissue by ultrasound waves generated by an ultrasound probe/transducer operatively connected to the equipment 100. Measurements and/or tracings of the heart 602 can be performed directly on the original ultrasound image displayed on the touch screen display 104 (see FIGS. 6A-6E), or on a magnified version of the ultrasound image. For example, using his or her fingers (see, e.g., fingers 610, 612; FIGS. 6B-6E), the user can perform a manual tracing of an endocardial border 604 (see FIG. 6B) of a left ventricle 606 (see FIGS. 6B-6E) of the heart 602 by employing one or more multi-finger gestures on the surface 105 of the touch screen display 104. In one embodiment, using his or her fingers (see, e.g., the fingers 610, 612; FIGS. 6B-6E), the user can obtain a cursor 607 (see FIG. 6B) by employing a double tap gesture (see, e.g., the double tap gesture 310; FIG. 3AA) on the surface 105 of the touch screen display 104, and can move the cursor 607 by employing a drag gesture (see, e.g., the drag gesture 318; FIG. 3AI) using one finger, such as the finger 610, thereby moving the cursor 607 to a desired location on the touch screen display 104. The systems and methods described herein can be used for the quantitative measurement of heart wall motion and specifically for the measurement of ventricular dysynchrony as described in detail in U.S. application Ser. No. 10/817,316 filed on Apr. 2, 2004, the entire contents of which is incorporated herein by reference.

Once the cursor 607 is at the desired location on the touch screen display 104, as determined by the location of the finger 610, the user can fix the cursor 607 at that location by employing a tap gesture (see, e.g., the tap gesture 302; see FIG. 3AA) using another finger, such as the finger 612. To perform a manual tracing of the endocardial border 604 (see FIG. 6B), the user can employ a press and drag gesture (see, e.g., the press and drag gesture 322; FIG. 3AK) using the finger 610, as illustrated in FIGS. 6C and 6D. Such a manual tracing of the endocardial border 604 can be highlighted on the touch screen display 104 in any suitable fashion, such as by a dashed line 608 (see FIGS. 6C-6E). The manual tracing of the endocardial border 604 can continue until the finger 610 arrives at any suitable location on the touch screen display 104, or until the finger 610 returns to the location of the cursor 607, as illustrated in FIG. 6E. Once the finger 610 is at the location of the cursor 607, or at any other suitable location, the user can complete the manual tracing operation by employing a tap gesture (see, e.g., the tap gesture 302; see FIG. 3AA) using the finger 612. It is noted that such a manual tracing operation can be employed to trace any other suitable feature(s) and/or waveform(s), such as a pulsed wave Doppler (PWD) waveform. In one embodiment, the medical ultrasound imaging equipment 100 can be configured to perform any suitable calculation(s) and/or measurement(s) relating to such feature(s) and/or waveform(s), based at least in part on a manual tracing(s) of the respective feature(s)/waveform(s).

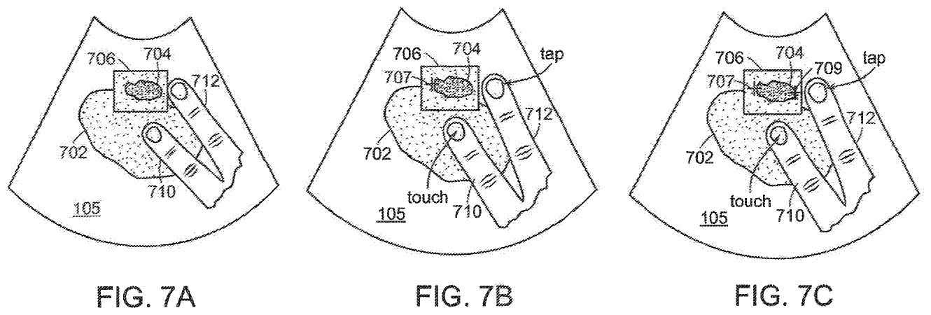

As described above, the user can perform measurements and/or tracings of objects on a magnified portion of an original ultrasound image of a displayed object within a virtual window on the touch screen display 104. FIGS. 7A-7C depict an original ultrasound image of an exemplary object, namely, a liver 702 with a cystic lesion 704, displayed on the touch screen display 104 of the medical ultrasound imaging equipment 100 (see FIG. 1). FIGS. 7A-7C further depict a virtual window 706 that provides a view of a magnified portion of the ultrasound image of the cystic lesion 704, which is covered by one of the user's fingers, such as a finger 710, pressed against the surface 105 of the touch screen display 104. Using his or her fingers (see, e.g., fingers 710, 712; FIGS. 7A-7C), the user can perform a size measurement of the cystic lesion 704 within the virtual window 706 by employing one or more multi-finger gestures on the surface 105 of the touch screen display 104.

For example, using his or her fingers (see, e.g., the fingers 710, 712; FIGS. 7A-7C), the user can obtain a first cursor 707 (see FIGS. 7B, 7C) by employing a double tap gesture (see, e.g., the double tap gesture 310; FIG. 3AE) on the surface 105, and can move the first cursor 707 by employing a drag gesture (see, e.g., the drag gesture 318; FIG. 3AI) using one finger, such as the finger 710, thereby moving the first cursor 707 to a desired location. Once the first cursor 707 is at the desired location, as determined by the location of the finger 710, the user can fix the first cursor 707 at that location by employing a tap gesture (see, e.g., the tap gesture 302; see FIG. 3AA) using another finger, such as the finger 712. Similarly, the user can obtain a second cursor 709 (see FIG. 7C) by employing a double tap gesture (see, e.g., the double tap gesture 310; FIG. 3AE) on the surface 105, and can move the second cursor 709 by employing a drag gesture (see, e.g., the drag gesture 318; FIG. 3AI) using the finger 710, thereby moving the second cursor 709 to a desired location. Once the second cursor 709 is at the desired location, as determined by the location of the finger 710, the user can fix the second cursor 709 at that location by employing a tap gesture (see, e.g., the tap gesture 302; see FIG. 3AA) using the finger 712. In one embodiment, the medical ultrasound imaging equipment 100 can be configured to perform any suitable size calculation(s) and/or measurement(s) relating to the cystic lesion 704, based at least in part on the locations of the first and second cursors 707, 709.

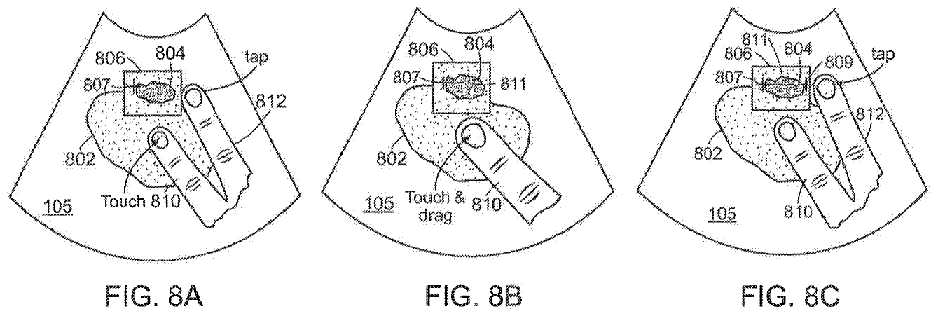

FIGS. 8A-8C depict an original ultrasound image of an exemplary object, namely, a liver 802 with a cystic lesion 804, displayed on the touch screen display 104 of the medical ultrasound imaging equipment 100 (see FIG. 1). FIGS. 8a-8c further depict a virtual window 806 that provides a view of a magnified portion of the ultrasound image of the cystic lesion 804, which is covered by one of the user's fingers, such as a finger 810, pressed against the surface 105 of the touch screen display 104. Using his or her fingers (see, e.g., fingers 810, 812; FIGS. 8A-8C), the user can perform a caliper measurement of the cystic lesion 804 within the virtual window 806 by employing one or more multi-finger gestures on the surface 105 of the touch screen display 104.

For example, using his or her fingers (see, e.g., the fingers 810, 812; FIGS. 8A-8C), the user can obtain a first cursor 807 (see FIGS. 8B, 8C) by employing a double tap gesture (see, e.g., the double tap gesture 310; FIG. 3) on the surface 105, and can move the cursor 807 by employing a drag gesture (see, e.g., the drag gesture 318; FIG. 3AI) using one finger, such as the finger 810, thereby moving the cursor 807 to a desired location. Once the cursor 807 is at the desired location, as determined by the location of the finger 810, the user can fix the cursor 807 at that location by employing a tap gesture (see, e.g., the tap gesture 302; see FIG. 3AA) using another finger, such as the finger 812. The user can then employ a press and drag gesture (see, e.g., the press and drag gesture 322; FIG. 3AK) to obtain a connecting line 811 (see FIGS. 8B, 8C), and to extend the connecting line 811 from the first cursor 807 across the cystic lesion 804 to a desired location on another side of the cystic lesion 804. Once the connecting line 811 is extended across the cystic lesion 804 to the desired location on the other side of the cystic lesion 804, the user can employ a tap gesture (see, e.g., the tap gesture 302; see FIG. 3AA) using the finger 812 to obtain and fix a second cursor 809 (see FIG. 8C) at that desired location. In one embodiment, the medical ultrasound imaging equipment 100 can be configured to perform any suitable caliper calculation(s) and/or measurement(s) relating to the cystic lesion 804, based at least in part on the connecting line 811 extending between the locations of the first and second cursors 807, 809.

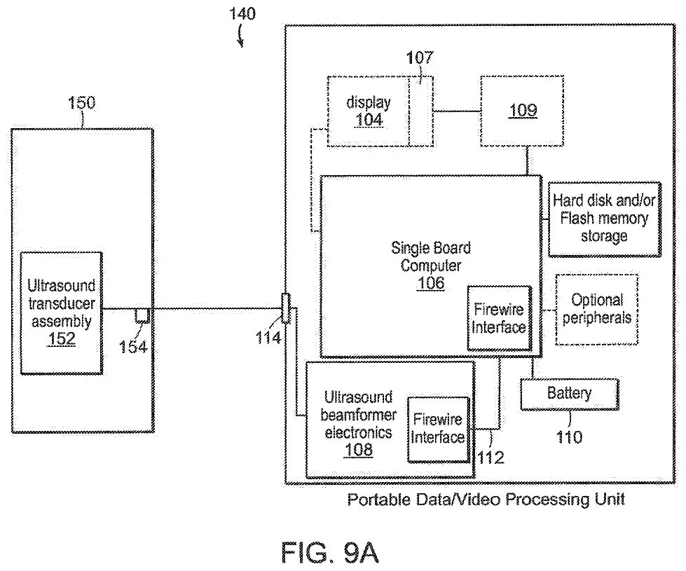

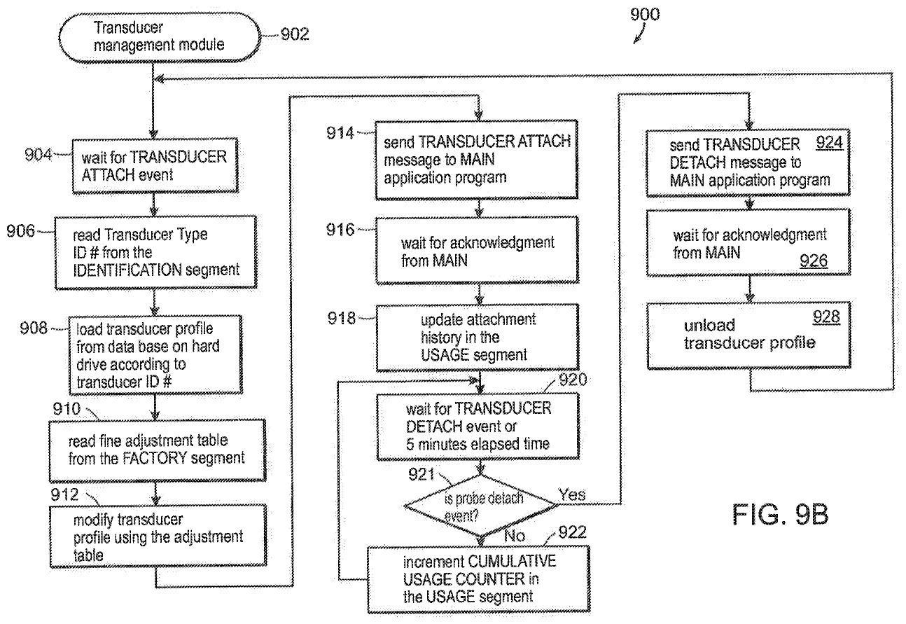

FIG. 9A shows a system 140 in which a transducer housing 150 with an array of transducer elements 152 can be attached at connector 114 to housing 102. Each probe 150 can have a probe identification circuit 154 that uniquely identifies the probe that is attached. When the user inserts a different probe with a different array, the system identifies the probe operating parameters. Note that preferred embodiments can include a display 104 having a touch sensor 107 which can be connected to a touch processor 109 that analyzes touchscreen data from the sensor 107 and transmits commands to both image processing operations and to a beamformer control processor (1116, 1124). In a preferred embodiment, the touch processor can include a computer readable medium that stores instructions to operate an ultrasound touchscreen engine that is operable to control display and imaging operations described herein.

FIG. 9B shows a software flowchart 900 of a typical transducer management module 902 within the ultrasound application program. When a TRANSDUCER ATTACH 904 event is detected, the Transducer Management Software Module 902 first reads the Transducer type ID 906 and hardware revision information from the IDENTIFICATION Segment. The information is used to fetch the particular set of transducer profile data 908 from the hard disk and load it into the memory of the application program. The software then reads the adjustment data from the FACTORY Segment 910 and applies the adjustments to the profile data just loaded into memory 912. The software module then sends a TRANSDUCER ATTACH Message 914 to the main ultrasound application program, which uses the transducer profile already loaded. After acknowledgment 916, an ultrasound imaging sequence is performed and the USAGE segment is updated 918. The Transducer Management Software Module then waits for either a TRANSDUCER DETACH event 920, or the elapse of 5 minutes. If a TRANSDUCER DETACH event is detected 921, a message 924 is sent and acknowledged 926, the transducer profile data set is removed 928 from memory and the module goes back to wait for another TRANSDUCER ATTACH event. If a 5 minutes time period expires without detecting a TRANSDUCER DETACH event, the software module increments a Cumulative Usage Counter in the USAGE Segment 922, and waits for another 5 minutes period or a TRANSDUCER DETACH event. The cumulative usage is recorded in memory for maintenance and replacement records.

There are many types of ultrasound transducers. They differ by geometry, number of elements, and frequency response. For example, a linear array with center frequency of 10 to 15 MHz is better suited for breast imaging, and a curved array with center frequency of 3 to 5 MHz is better suited for abdominal imaging.

It is often necessary to use different types of transducers for the same or different ultrasound scanning sessions. For ultrasound systems with only one transducer connection, the operator will change the transducer prior to the start of a new scanning session.

In some applications, it is necessary to switch among different types of transducers during one ultrasound scanning session. In this case, it is more convenient to have multiple transducers connected to the same ultrasound system, and the operator can quickly switch among these connected transducers by hitting a button on the operator console, without having to physically detach and re-attach the transducers, which takes a longer time. Preferred embodiments of the invention can include a multiplexor within the tablet housing that can select between a plurality of probe connector ports within the tablet housing, or alternatively, the tablet housing can be connected to an external multiplexor that can be mounted on a cart as described herein.

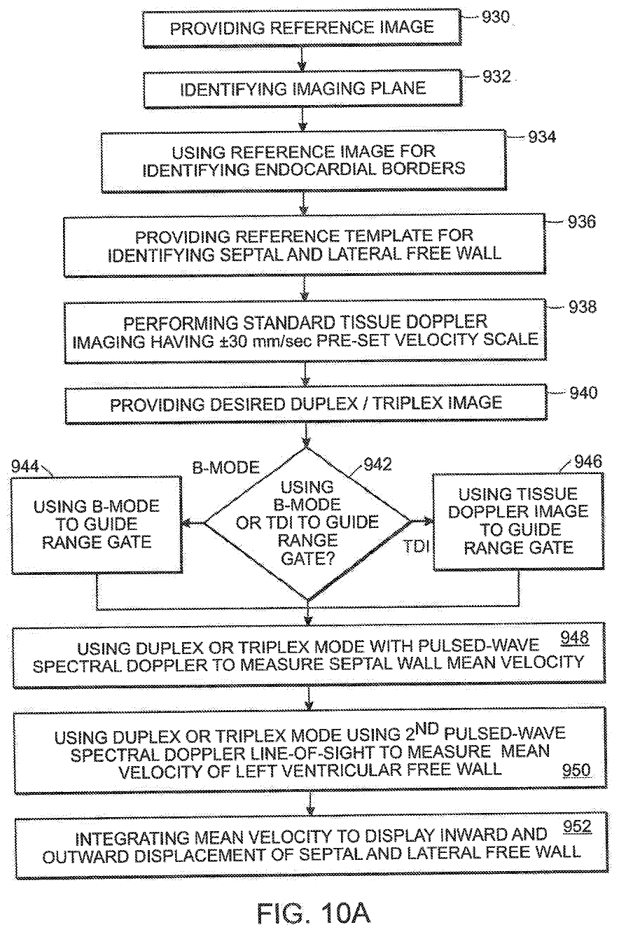

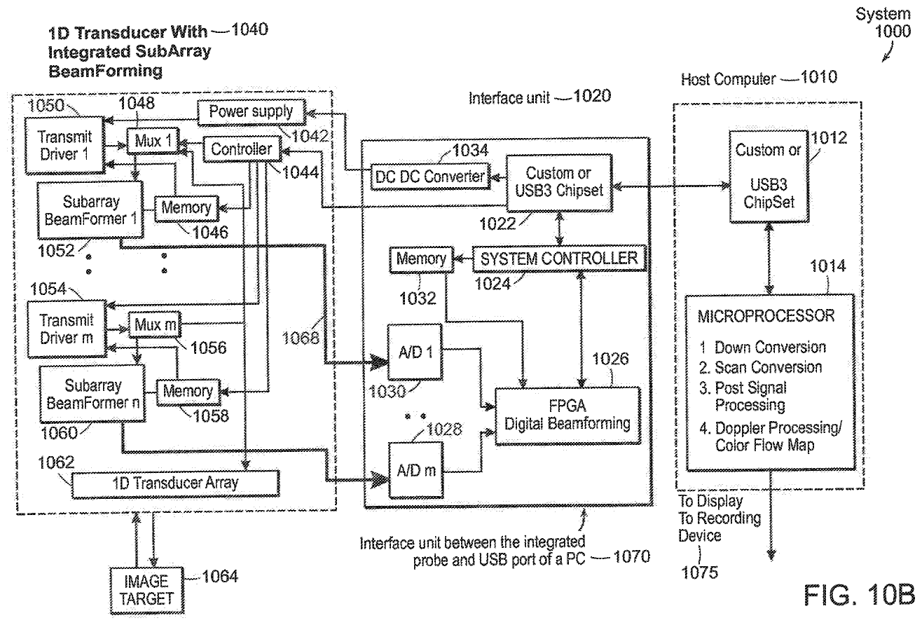

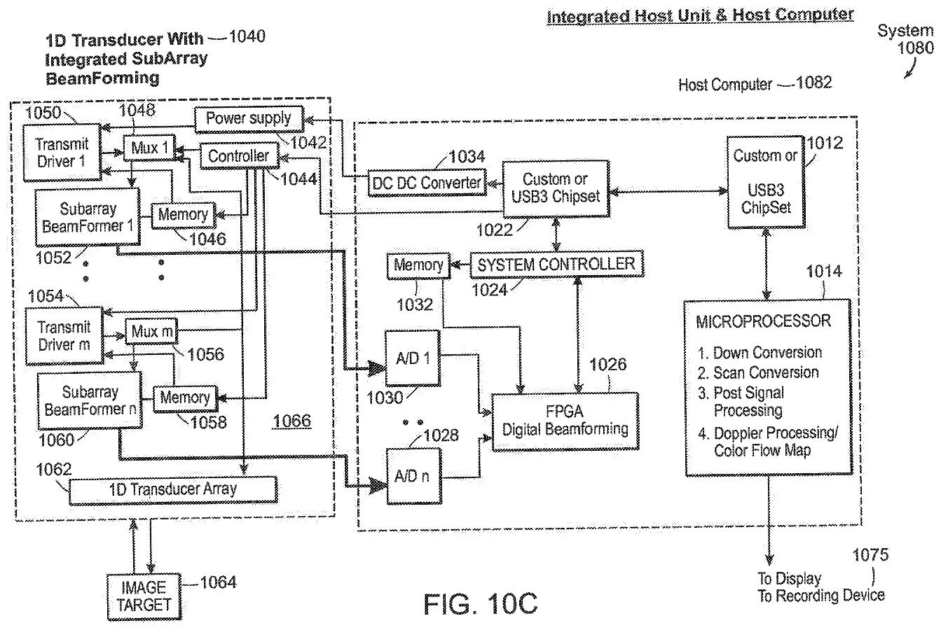

FIG. 10A illustrates an exemplary method for monitoring the synchrony of a heart in accordance with exemplary embodiments. In the method, a reference template is loaded into memory and used to guide a user in identifying an imaging plane (per step 930). Next a user identifies a desired imaging plane (per step 932). Typically an apical 4-chamber view of the heart is used; however, other views may be used without departing from the spirit of the invention.

At times, identification of endocardial borders may be difficult, and when such difficulties are encountered tissue Doppler imaging of the same view may be employed (per step 934). A reference template for identifying the septal and lateral free wall is provided (per step 936). Next, standard tissue Doppler imaging (TDI) with pre-set velocity scales of, say, ±30 cm/sec may be used (per step 938).

Then, a reference of the desired triplex image may be provided (per step 940). Either B-mode or TDI may be used to guide the range gate (per step 942). B-mode can be used for guiding the range gate (per step 944) or TDI for guiding the range gate (per step 946). Using TDI or B-mode for guiding the range gate also allows the use of a direction correction angle for allowing the Spectral Doppler to display the radial mean velocity of the septal wall. A first pulsed-wave spectral Doppler is then used to measure the septal wall mean velocity using duplex or triplex mode (per step 948). The software used to process the data and calculate dysychrony can utilize a location (e.g. a center point) to automatically set an angle between dated locations on a heart wall to assist in simplifying the setting of parameters.

A second range-gate position is also guided using a duplex image or a TDI (per step 950), and a directional correction angle may be used if desired. After step 950, the mean velocity of the septal wall and lateral free wall are being tracked by the system. Time integration of the Spectral Doppler mean velocities 952 at regions of interest (e.g., the septum wall and the left ventricular free wall) then provides the displacement of the septal and left free wall, respectively.

The above method steps may be utilized in conjunction with a high pass filtering means, analog or digital, known in the relevant arts for removing any baseline disturbance present in collected signals. In addition, the disclosed method employs multiple simultaneous PW Spectral Doppler lines for tracking movement of the interventricular septum and the left ventricular free wall. In addition, a multiple gate structure may be employed along each spectral line, thus allowing quantitative measurement of regional wall motion. Averaging over multiple gates may allow measurement of global wall movement.

FIG. 10B is a detailed schematic block diagram 1000 for an exemplary embodiment of the integrated ultrasound probe 1040 that can be connected to any PC 1010 through an Interface unit 1020. The ultra sound probe 1040 is configured to transmit ultrasound waves to and reduce reflected ultrasound waves from one or more image targets 1064. The transducer 1040 can be coupled to the interface unit 1020 using one or more cables 1066, 1068. The interface unit 1020 can be positioned between the integrated ultrasound probe 1040 and the host computer 1010. The two stage beam forming system 1040 and 1020 can be connected to any PC through a USB connection 1022, 1012.

The ultrasound probe 1040, can include sub-arrays/apertures 1052 consisting of neighboring elements with an aperture smaller than that of the whole array. Returned echoes are received by the 1D transducer array 1062 and transmitted to the controller 1044. The controller initiates formation of a coarse beam by transmitting the signals to memory 1058, 1046. The memory 1058, 1046 transmits a signal to a transmit Driver 1 1050, and Transmit Driver m 1054. Transmit Driver 1 1050 and Transmit Driver m 1054 then send the signal to mux1 1048 and mux m 1056, respectively. The signal is transmitted to sub-array beamformer 1 1052 and sub-array beamformer n 1060.

The outputs of each coarse beam forming operation can include further processing through a second stage beam forming in the interface unit 1020 to convert the beam forming output to digital representation. The coarse beam forming operations can be coherently summed to form a fine beam output for the array. The signals can be transmitted from the ultrasound probe 1040 sub-array beam former 1 1052 and sub-array beam former n 1060 to the A/D convertors 1030 and 1028 within the interface unit 1020. Within the interface unit 1020 there are A/D converters 1028, 1030 for converting the first stage beam forming output to digital representation. The digital conversion can be received from the A/D convertors 1030, 1028 by a customer ASIC such as a FPGA 1026 to complete the second stage beam forming. The FPGA Digital beam forming 1026 can transmit information to the system controller 1024. The system controller can transmit information to a memory 1032 which may send a signal back to the FPGA Digital Beam forming 1026. Alternatively, the system controller 1024 may transmit information to the custom USB3 Chipset 1022. The USB3 Chipset 1022 may then transmit information to a DC-DC convertor 1034. In turn, the DC-DC convertor 1034 may transmit power from the interface unit 1020 to the ultrasound probe 1040. Within the ultrasound probe 1040 a power supply 1042 may receive the power signal and interface with the transmit driver 1 1050 to provide the power to the front end integration probe.

The Interface unit 1020 custom or USB3 Chipset 1022 may be used to provide a communication link between the interface unit 1020 and the host computer 1010. The custom or USB3 Chipset 1022 transmits a signal to the host computer's 1010 custom or USB3 Chipset 1012. The custom or the USB3 Chipset 1012 then interfaces with the microprocessor 1014. The microprocessor 1014 then may display information or send information to a device 1075.

In an alternate embodiment, a narrow band beamformer can be used. For example, an individual analog phase shifter is applied to each of the received echoes. The phase shifted outputs within each sub-array are then summed to form a coarse beam. The A/D converts can be used to digitize each of the coarse beams; a digital beam former is then used to form the fine beam.

In another embodiment, forming a 64 element linear array may use eight adjacent elements to form a coarse beam output. Such arrangement may utilize eight output analog cables connecting the outputs of the integrated probe to the interface units. The coarse beams may be sent through the cable to the corresponding A/D convertors located in the interface unit. The digital delay is used to form a fine beam output. Eight A/D convertors may be required to form the digital representation.

In another embodiment, forming a 128 element array may use sixteen sub-array beam forming circuits. Each circuit may form a coarse beam from an adjacent eight element array provided in the first stage output to the interface unit. Such arrangement may utilize sixteen output analog cables connecting the outputs of the integrated probe to the interface units to digitize the output. A PC microprocessor or a DSP may be used to perform the down conversion, base-banding, scan conversion and post image processing functions. The microprocessor or DSP can also be used to perform all the Doppler processing functions.

FIG. 10C is a detailed schematic block diagram 1080 for an exemplary embodiment of the integrated ultrasound probe 1040 with the first sub array beamforming circuit, and the second stage beamforming circuits are integrated inside the host computer 1082. The back end computer with the second stage beamforming circuit may be a PDA, tablet or mobile device housing. The ultra sound probe 1040 is configured to transmit ultrasound waves to and reduce reflected ultrasound waves from one or more image targets 1064. The transducer 1040 is coupled to the host computer 1082 using one or more cables 1066, 1068. Note that A/D circuit elements can also be placed in the transducer probe housing.

The ultrasound probe 1040 includes subarray/apertures 1052 consisting of neighboring elements with an aperture smaller than that of the whole array. Returned echoes are received by the 1D transducer array 1062 and transmitted to the controller 1044. The controller initiates formation of a coarse beam by transmitting the signals to memory 1058, 1046. The memory 1058, 1046 transmits a signal to a transmit Driver 1 1050, and Transmit Driver m 1054. Transmit Driver 1 1050 and Transmit Driver m 1054 then send the signal to mux1 1048 and mux m 1056, respectively. The signal is transmitted to subarray beamformer 1 1052 and subarray beamformer n 1060.

The outputs of each coarse beam forming operation then go through a second stage beam forming in the interface unit 1020 to convert the beam forming output to digital representation. The coarse beamforming operations are coherently summed to form a fine beam output for the array. The signals are transmitted from the ultrasound probe 1040 subarray beamformer 1 1052 and subarray beamformer n 1060 to the A/D convertors 1030 and 1028 within the host computer 1082. Within the host computer 1082 there are A/D converters 1028, 1030 for converting the first stage beamforming output to digital representation. The digital conversion is received from the A/D convertors 1030, 1028 by a customer ASIC such as a FPGA 1026 to complete the second stage beamforming. The FPGA Digital beamforming 1026 transmits information to the system controller 1024. The system controller transmits information to a memory 1032 which may send a signal back to the FPGA Digital Beam forming 1026. Alternatively, the system controller 1024 may transmit information to the custom USB3 Chipset 1022. The USB3 Chipset 1022 may then transmit information to a DC-DC convertor 1034. In turn, the DC-DC convertor 1034 may transmit power from the interface unit 1020 to the ultrasound probe 1040. Within the ultrasound probe 1040 a power supply 1042 may receive the power signal and interface with the transmit driver 1 1050 to provide the power to the front end integration probe. The power supply can include a battery to enable wireless operation of the transducer assembly. A wireless transceiver can be integrated into controller circuit or a separate communications circuit to enable wireless transfer of image data and control signals.

The host computer's 1082 custom or USB3 Chipset 1022 may be used to provide a communication link between the custom or USB3 Chipset 1012 to transmits a signal to the microprocessor 1014. The microprocessor 1014 then may display information or send information to a device 1075.

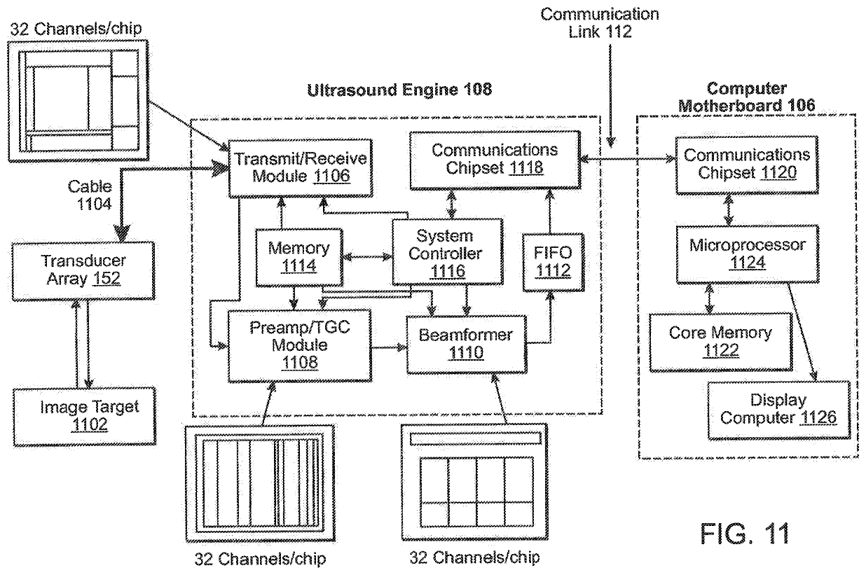

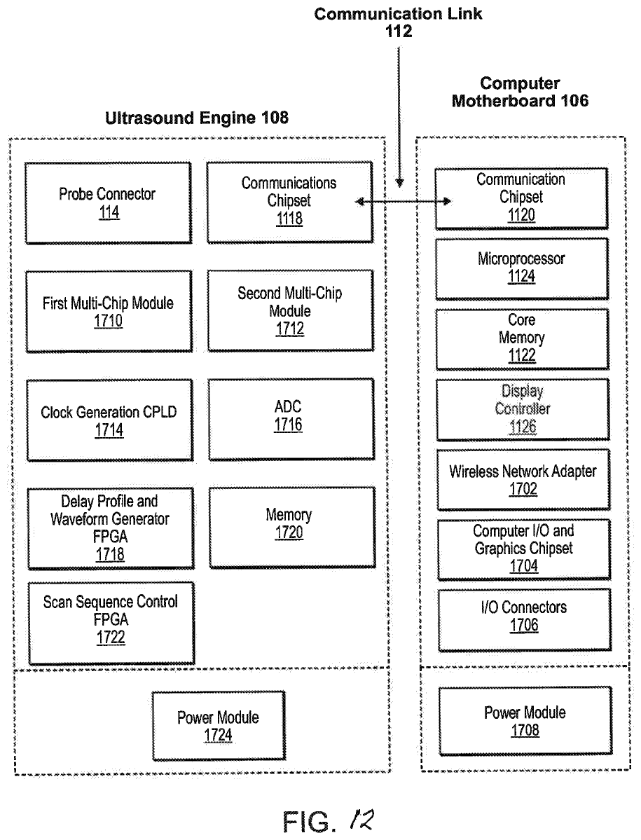

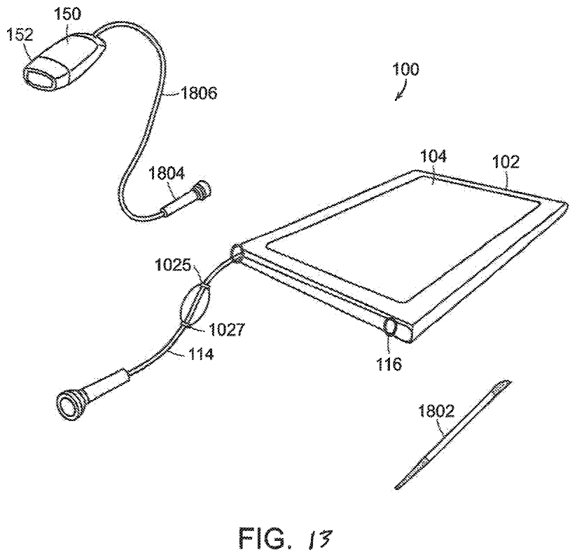

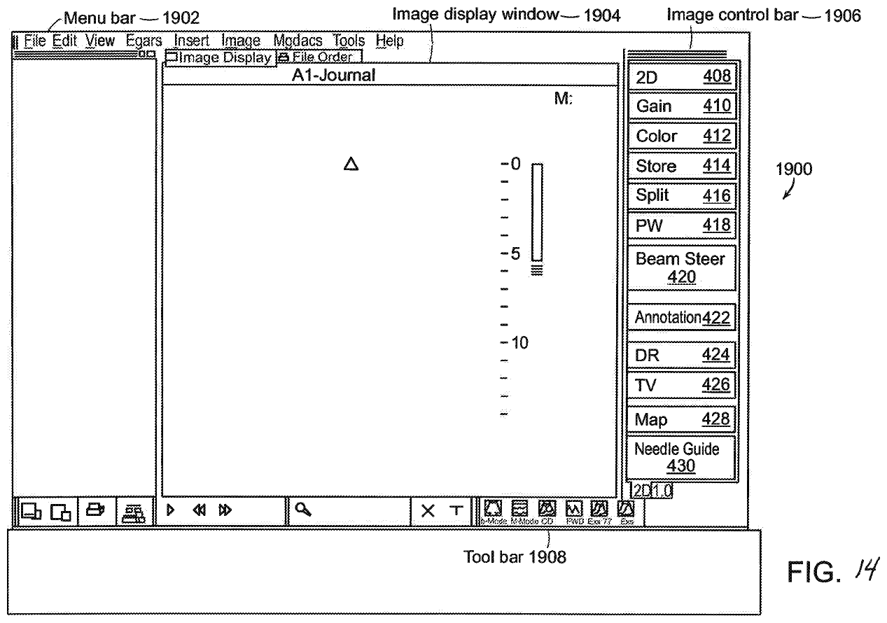

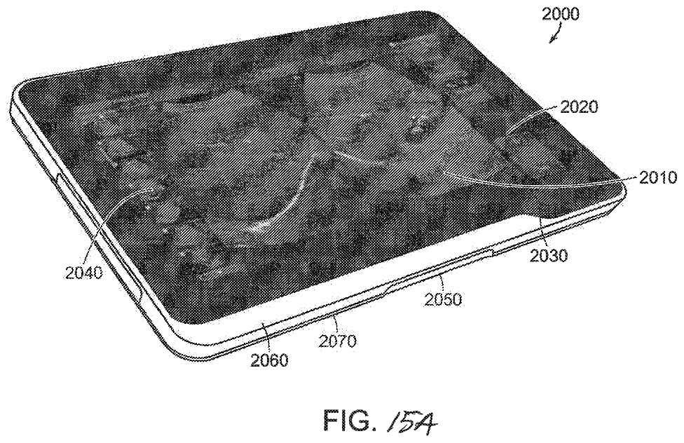

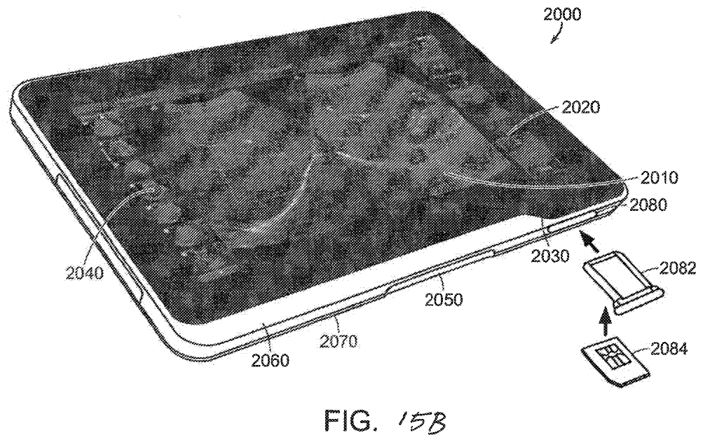

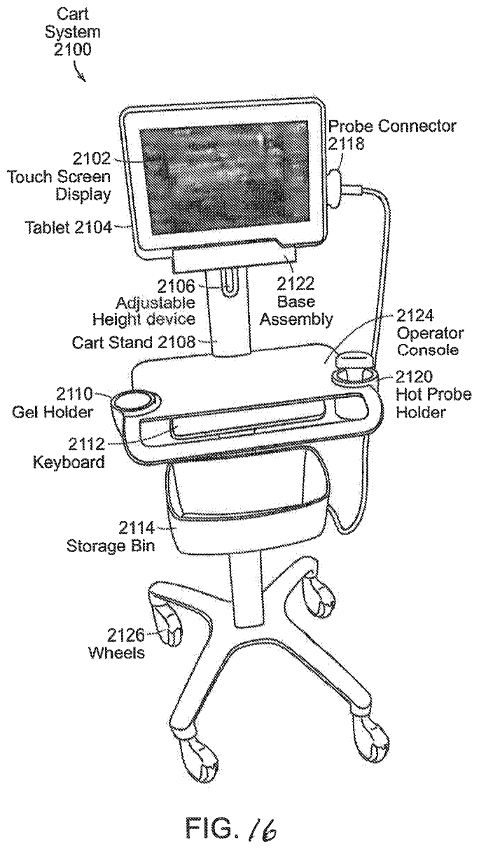

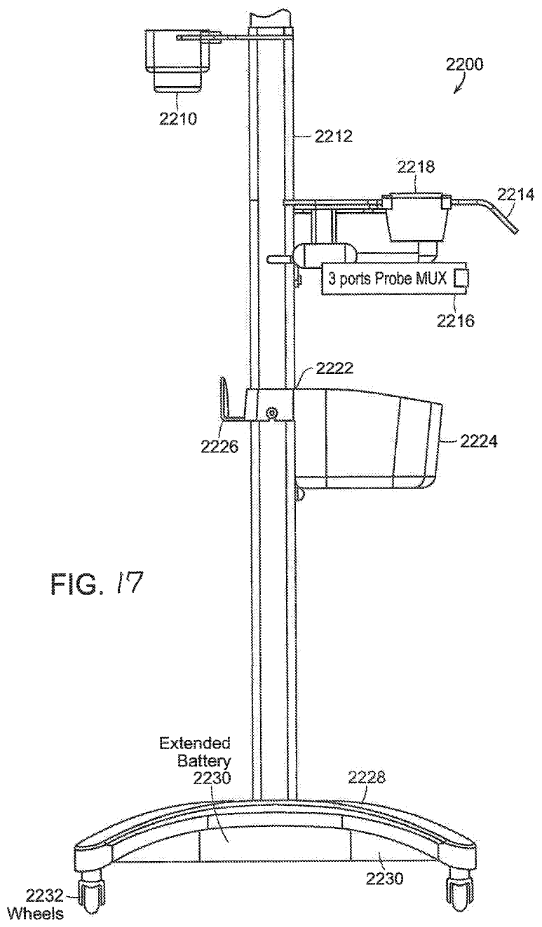

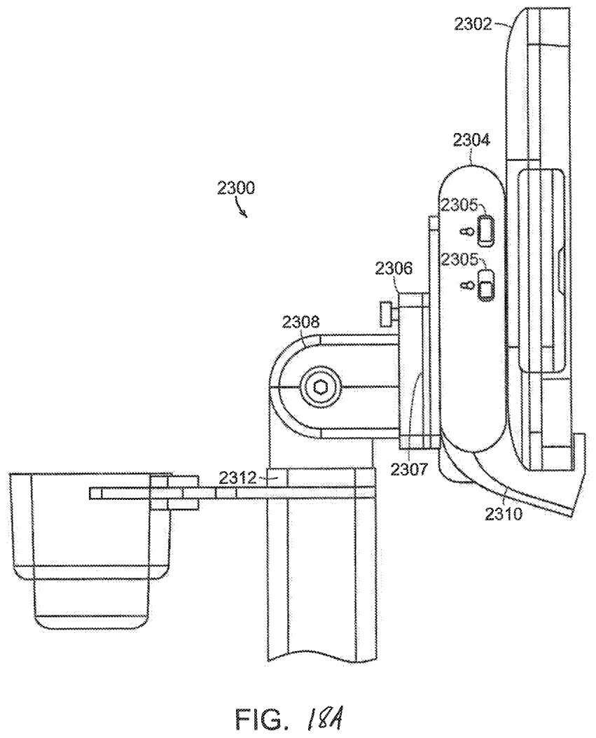

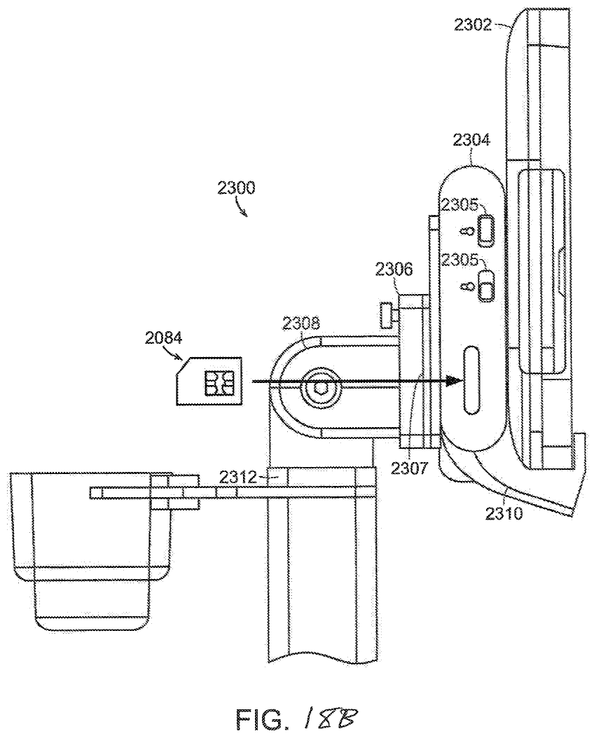

FIG. 11 is a detailed schematic block diagram of an exemplary embodiment of the ultrasound engine 108 (i.e., the front-end ultrasound specific circuitry) and an exemplary embodiment of the computer motherboard 106 (i.e., the host computer) of the ultrasound device illustrated in FIGS. 1A and 2A. The components of the ultrasound engine 108 and/or the computer motherboard 106 may be implemented in application-specific integrated circuits (ASICs). Exemplary ASICs have a high channel count and can pack 32 or more channels per chip in some exemplary embodiments. One of ordinary skill in the art will recognize that the ultrasound engine 108 and the computer motherboard 106 may include more or fewer modules than those shown. For example, the ultrasound engine 108 and the computer motherboard 106 may include the modules shown in FIG. 17.

A transducer array 152 is configured to transmit ultrasound waves to and receive reflected ultrasound waves from one or more image targets 1102. The transducer array 152 is coupled to the ultrasound engine 108 using one or more cables 1104.

The ultrasound engine 108 includes a high-voltage transmit/receive (TR) module 1106 for applying drive signals to the transducer array 152 and for receiving return echo signals from the transducer array 152. The ultrasound engine 108 includes a pre-amp/time gain compensation (TGC) module 1108 for amplifying the return echo signals and applying suitable TGC functions to the signals. The ultrasound engine 108 includes a sampled-data beamformer 1110 by which the delay coefficients used in each channel thereof after the return echo signals have been amplified and processed by the pre-amp/TGC module 1108.

In some exemplary embodiments, the high-voltage TR module 1106, the pre-amp/TGC module 1108, and the sample-interpolate receive beamformer 1110 may each be a silicon chip having 8 to 64 channels per chip, but exemplary embodiments are not limited to this range. In certain embodiments, the high-voltage TR module 1106, the pre-amp/TGC module 1108, and the sample-interpolate receive beamformer 1110 may each be a silicon chip having 8, 16, 32, 64 channels, and the like. As illustrated in FIG. 11, an exemplary TR module 1106, an exemplary pre-amp/TGC module 1108 and an exemplary beamformer 1110 may each take the form of a silicon chip including 32 channels.

The ultrasound engine 108 includes a first-in first-out (FIFO) buffer module 1112 which is used for buffering the processed data output by the beamformer 1110. The ultrasound engine 108 also includes a memory 1114 for storing program instructions and data, and a system controller 1116 for controlling the operations of the ultrasound engine modules.

The ultrasound engine 108 interfaces with the computer motherboard 106 over a communications link 112 which can follow a standard high-speed communications protocol, such as the Fire Wire (IEEE 1394 Standards Serial Interface) or fast (e.g., 200-400 Mbits/second or faster) Universal Serial Bus (USB 2.0 USB 3.0), protocol. The standard communication link to the computer motherboard operates at least at 400 Mbits/second or higher, preferably at 800 Mbits/second or higher. Alternatively, the link 112 can be a wireless connection such as an infrared (IR) link. The ultrasound engine 108 includes a communications chipset 1118 (e.g., a Fire Wire chipset) to establish and maintain the communications link 112. Similarly, the computer motherboard 106 also includes a communications chipset 1120 (e.g., a Fire Wire chipset) to establish and maintain the communications link 112. The computer motherboard 106 includes a core computer-readable memory 1122 for storing data and/or computer-executable instructions for performing ultrasound imaging operations. The memory 1122 forms the main memory for the computer and, in an exemplary embodiment, may store about 4 GB of DDR3 memory. The computer motherboard 106 also includes a microprocessor 1124 for executing computer-executable instructions stored on the core computer-readable memory 1122 for performing ultrasound imaging processing operations. An exemplary microprocessor 1124 may be an off-the-shelf commercial computer processor, such as an Intel Core-i5 processor. Another exemplary microprocessor 1124 may be a digital signal processor (DSP) based processor, such as one or more DaVinci™ processors from Texas Instruments. The computer motherboard 106 also includes a display controller 1126 for controlling a display device that may be used to display ultrasound data, scans and maps.

Exemplary operations performed by the microprocessor 1124 include, but are not limited to, down conversion (for generating I, Q samples from received ultrasound data), scan conversion (for converting ultrasound data into a display format of a display device), Doppler processing (for determining and/or imaging movement and/or flow information from the ultrasound data), Color Flow processing (for generating, using autocorrelation in one embodiment, a color-coded map of Doppler shifts superimposed on a B-mode ultrasound image), Power Doppler processing (for determining power Doppler data and/or generating a power Doppler map), Spectral Doppler processing (for determining spectral Doppler data and/or generating a spectral Doppler map), and post signal processing. These operations are described in further detail in WO 03/079038 A2, filed Mar. 11, 2003, titled “Ultrasound Probe with Integrated Electronics,” the entire contents of which are expressly incorporated herein by reference.

To achieve a smaller and lighter portable ultrasound devices, the ultrasound engine 108 includes reduction in overall packaging size and footprint of a circuit board providing the ultrasound engine 108. To this end, exemplary embodiments provide a small and light portable ultrasound device that minimizes overall packaging size and footprint while providing a high channel count. In some embodiments, a high channel count circuit board of an exemplary ultrasound engine may include one or more multi-chip modules in which each chip provides multiple channels, for example, 32 channels. The term “multi-chip module,” as used herein, refers to an electronic package in which multiple integrated circuits (IC) are packaged into a unifying substrate, facilitating their use as a single component, i.e., as a larger IC. A multi-chip module may be used in an exemplary circuit board to enable two or more active IC components integrated on a High Density Interconnection (HDI) substrate to reduce the overall packaging size. In an exemplary embodiment, a multi-chip module may be assembled by vertically stacking a transmit/receive (TR) silicon chip, an amplifier silicon chip and a beamformer silicon chip of an ultrasound engine. A single circuit board of the ultrasound engine may include one or more of these multi-chip modules to provide a high channel count, while minimizing the overall packaging size and footprint of the circuit board.

FIG. 12 is a detailed schematic block diagram of an exemplary embodiment of the ultrasound engine 108 (i.e., the front-end ultrasound specific circuitry) and an exemplary embodiment of the computer motherboard 106 (i.e., the host computer) provided as a single board complete ultrasound system. An exemplary single board ultrasound system as illustrated in FIG. 12 may have exemplary planar dimensions of about 25 cm×about 18 cm, although other dimensions are possible. The single board complete ultrasound system of FIG. 17 may be implemented in the ultrasound device illustrated in FIGS. 1A, 2A, 2B, and 9A, and may be used to perform the operations depicted in FIGS. 3A-8C, 9B, and 10A.

The ultrasound engine 108 includes a probe connector 114 to facilitate the connection of at least one ultrasound probe/transducer. In the ultrasound engine 108, a TR module, an amplifier module and a beamformer module may be vertically stacked to form a multi-chip module, thereby minimizing the overall packaging size and footprint of the ultrasound engine 108. The ultrasound engine 108 may include a first multi-chip module 1710 and a second multi-chip module 1712, each including a TR chip, an ultrasound pulser and receiver, an amplifier chip including a time-gain control amplifier, and a sample-data beamformer chip vertically integrated in a stacked configuration. The first and second multi-chip modules 1710, 1712 may be stacked vertically on top of each other to further minimize the area required on the circuit board. Alternatively, the first and second multi-chip modules 1710, 1712 may be disposed horizontally on the circuit board. In an exemplary embodiment, the TR chip, the amplifier chip and the beamformer chip is each a 32-channel chip, and each multi-chip module 1710, 1712 has 32 channels. One of ordinary skill in the art will recognize that exemplary ultrasound engines 108 may include, but are not limited to, one, two, three, four, five, six, seven, eight multi-chip modules. Note that in a preferred embodiment the system can be configured with a first beamformer in the transducer housing and a second beamformer in the tablet housing.

The ASICs and the multi-chip module configuration enable a 128-channel complete ultrasound system to be implemented on a small single board in a size of a tablet computer format. An exemplary 128-channel ultrasound engine 108, for example, can be accommodated within exemplary planar dimensions of about 10 cm×about 10 cm, which is a significant improvement of the space requirements of conventional ultrasound circuits. An exemplary 128-channel ultrasound engine 108 can also be accommodated within an exemplary area of about 100 cm².

The ultrasound engine 108 also includes a clock generation complex programmable logic device (CPLD) 1714 for generating timing clocks for performing an ultrasound scan using the transducer array. The ultrasound engine 108 includes an analog-to-digital converter (ADC) 1716 for converting analog ultrasound signals received from the transducer array to digital RF formed beams. The ultrasound engine 108 also includes one or more delay profile and waveform generator field programmable gate arrays (FPGA) 1718 for managing the receive delay profiles and generating the transmit waveforms. The ultrasound engine 108 includes a memory 1720 for storing the delay profiles for ultrasound scanning. An exemplary memory 1720 may be a single DDR3 memory chip. The ultrasound engine 108 includes a scan sequence control field programmable gate array (FPGA) 1722 configured to manage the ultrasound scan sequence, transmit/receiving timing, storing and fetching of profiles to/from the memory 1720, and buffering and moving of digital RF data streams to the computer motherboard 106 via a high-speed serial interface 112. The high-speed serial interface 112 may include Fire Wire or other serial or parallel bus interface between the computer motherboard 106 and the ultrasound engine 108. The ultrasound engine 108 includes a communications chipset 1118 (e.g., a Fire Wire chipset) to establish and maintain the communications link 112.

A power module 1724 is provided to supply power to the ultrasound engine 108, manage a battery charging environment and perform power management operations. The power module 1724 may generate regulated, low noise power for the ultrasound circuitry and may generate high voltages for the ultrasound transmit pulser in the TR module.

The computer motherboard 106 includes a core computer-readable memory 1122 for storing data and/or computer-executable instructions for performing ultrasound imaging operations. The memory 1122 forms the main memory for the computer and, in an exemplary embodiment, may store about 4 Gb of DDR3 memory. The memory 1122 may include a solid state hard drive (SSD) for storing an operating system, computer-executable instructions, programs and image data. An exemplary SSD may have a capacity of about 128 GB.

The computer motherboard 106 also includes a microprocessor 1124 for executing computer-executable instructions stored on the core computer-readable memory 1122 for performing ultrasound imaging processing operations. Exemplary operations include, but are not limited to, down conversion, scan conversion, Doppler processing, Color Flow processing, Power Doppler processing, Spectral Doppler processing, and post signal processing. An exemplary microprocessor 1124 may be an off-the-shelf commercial computer processor, such as an Intel Core-i5 processor. Another exemplary microprocessor 1124 may be a digital signal processor (DSP) based processor, such as DaVinci™ processors from Texas Instruments.

The computer motherboard 106 includes an input/output (I/O) and graphics chipset 1704 which includes a co-processor configured to control I/O and graphic peripherals such as USB ports, video display ports and the like. The computer motherboard 106 includes a wireless network adapter 1702 configured to provide a wireless network connection. An exemplary adapter 1702 supports 802.11g and 802.11n standards. The computer motherboard 106 includes a display controller 1126 configured to interface the computer motherboard 106 to the display 104. The computer motherboard 106 includes a communications chipset 1120 (e.g., a Fire Wire chipset or interface) configured to provide a fast data communication between the computer motherboard 106 and the ultrasound engine 108. An exemplary communications chipset 1120 may be an IEEE 1394b 800 Mbit/sec interface. Other serial or parallel interfaces 1706 may alternatively be provided, such as USB3, Thunder-Bolt, PCIe, and the like. A power module 1708 is provided to supply power to the computer motherboard 106, manage a battery charging environment and perform power management operations.

An exemplary computer motherboard 106 may be accommodated within exemplary planar dimensions of about 12 cm×about 10 cm. An exemplary computer motherboard 106 can be accommodated within an exemplary area of about 120 cm².

FIG. 13 is a perspective view of an exemplary portable ultrasound system 100 provided in accordance with exemplary embodiments. The system 100 includes a housing 102 that is in a tablet form factor as illustrated in FIG. 13, but that may be in any other suitable form factor. An exemplary housing 102 may have a thickness below 2 cm and preferably between 0.5 and 1.5 cm. A front panel of the housing 102 includes a multi-touch LCD touch screen display 104 that is configured to recognize and distinguish one or more multiple and/or simultaneous touches on a surface of the touch screen display 104. The surface of the display 104 may be touched using one or more of a user's fingers, a user's hand or an optional stylus 1802. The housing 102 includes one or more I/O port connectors 116 which may include, but are not limited to, one or more USB connectors, one or more SD cards, one or more network mini display ports, and a DC power input. The embodiment of housing 102 in FIG. 13 can also be configured within a palm-carried form factor having dimensions of 150 mm×100 mm×15 mm (a volume of 225000 mm³) or less. The housing 102 can have a weight of less than 200 g. Optionally, cabling between the transducer array and the display housing can include interface circuitry 1020 as described herein. The interface circuitry 1020 can include, for example, beamforming circuitry and/or A/D circuitry in a pod that dangles from the tablet. Separate connectors 1025, 1027 can be used to connect the dangling pod to the transducer probe cable. The connector 1027 can include probe identification circuitry as described herein. The housing 102 can include a camera, a microphone and a speaker as well as wireless telephone circuitry for voice and data communications as well as voice activated software that can be used to control the ultrasound imaging operations described herein.

The housing 102 includes or is coupled to a probe connector 114 to facilitate connection of at least one ultrasound probe/transducer 150. The ultrasound probe 150 includes a transducer housing including one or more transducer arrays 152. The ultrasound probe 150 is couplable to the probe connector 114 using a housing connector 1804 provided along a flexible cable 1806. One of ordinary skill in the art will recognize that the ultrasound probe 150 may be coupled to the housing 102 using any other suitable mechanism, for example, an interface housing that includes circuitry for performing ultrasound-specific operations like beamforming. Other exemplary embodiments of ultrasound systems are described in further detail in WO 03/079038 A2, filed Mar. 11, 2003, titled “Ultrasound Probe with Integrated Electronics,” the entire contents of which is expressly incorporated herein by reference. Preferred embodiments can employ a wireless connection between the hand-held transducer probe 150 and the display housing. Beamformer electronics can be incorporated into probe housing 150 to provide beamforming of subarrays in a 1D or 2D transducer array as described herein. The display housing can be sized to be held in the palm of the user's hand and can include wireless network connectivity to public access networks such as the internet.

FIG. 14 illustrates an exemplary view of a main graphical user interface (GUI) 1900 rendered on the touch screen display 104 of the portable ultrasound system 100 of FIG. 18. The main GUI 1900 may be displayed when the ultrasound system 100 is started. To assist a user in navigating the main GUI 1900, the GUI may be considered as including four exemplary work areas: a menu bar 1902, an image display window 1904, an image control bar 1906, and a tool bar 1908. Additional GUI components may be provided on the main GUI 1900 to, for example, enable a user to close, resize and exit the GUI and/or windows in the GUI.

The menu bar 1902 enables a user to select ultrasound data, images and/or videos for display in the image display window 1904. The menu bar 1902 may include, for example, GUI components for selecting one or more files in a patient folder directory and an image folder directory. The image display window 1904 displays ultrasound data, images and/or videos and may, optionally, provide patient information. The tool bar 1908 provides functionalities associated with an image or video display including, but not limited to, a save button for saving the current image and/or video to a file, a save Loop button that saves a maximum allowed number of previous frames as a Cine loop, a print button for printing the current image, a freeze image button for freezing an image, a playback toolbar for controlling aspects of playback of a Cine loop, and the like. Exemplary GUI functionalities that may be provided in the main GUI 1900 are described in further detail in WO 03/079038 A2, filed Mar. 11, 2003, titled “Ultrasound Probe with Integrated Electronics,” the entire contents of which are expressly incorporated herein by reference.

The image control bar 1906 includes touch controls that may be operated by touch and touch gestures applied by a user directly to the surface of the display 104. Exemplary touch controls may include, but are not limited to, a 2D touch control 408, a gain touch control 410, a color touch control 412, a storage touch control 414, a split touch control 416, a PW imaging touch control 418, a beamsteering touch control 420, an annotation touch control 422, a dynamic range operations touch control 424, a Teravision™ touch control 426, a map operations touch control 428, and a needle guide touch control 428. These exemplary touch controls are described in further detail in connection with FIGS. 4a-4c.

FIG. 15A depicts an illustrative embodiment of exemplary medical ultrasound imaging equipment 2000, implemented in the form factor of a tablet in accordance with the invention. The tablet may have the dimensions of 12.5″×1.25″×8.75″ or 31.7 cm×3.175 cm×22.22 cm but it may also be in any other suitable form factor having a volume of less than 2500 cm³ and a weight of less than 8 lbs. As shown in FIG. 15A-B, the medical ultrasound imaging equipment 2000, includes a housing 2030, a touch screen display 2010, wherein ultrasound images, and ultra sound data 2040, can be displayed and ultrasound controls 2020, are configured to be controlled by a touchscreen display 2010. The housing 2030, may have a front panel 2060 and a rear panel 2070. The touchscreen display 2010, forms the front panel 2060, and includes a multi-touch LCD touch screen that can recognize and distinguish one or more multiple and or simultaneous touches of the user on the touchscreen display 2010. The touchscreen display 2010 may have a capacitive multi-touch and AVAH LCD screen. For example, the capacitive multi-touch and AVAH LCD screen may enable a user to view the image from multi angles without losing resolution. In another embodiment, the user may utilize a stylus for data input on the touch screen. The tablet can include an integrated foldable stand that permits a user to swivel the stand from a storage position that conforms to the tablet form factor so that the device can lay flat on the rear panel, or alternatively, the user can swivel the stand to enable the tablet to stand at an upright position at one of a plurality of oblique angles relative to a support surface. Capacitive touchscreen module comprises an insulator for example glass, coated with a transparent conductor, such as indium tin oxide. The manufacturing process may include a bonding process among glass, x-sensor film, y-sensor film and a liquid crystal material. The tablet is configured to allow a user to perform multi-touch gestures such as pinching and stretching while wearing a dry or a wet glove. The surface of the screen registers the electrical conductor making contact with the screen. The contact distorts the screens electrostatic field resulting in measureable changes in capacitance. A processor then interprets the change in the electrostatic field. Increasing levels of responsiveness are enabled by reducing the layers and by producing touch screens with “in-cell” technology. “In-cell” technology eliminates layers by placing the capacitors inside the display. Applying “in-cell” technology reduces the visible distance between the user's finger and the touchscreen target, thereby creating a more directive contact with the content displayed and enabling taps and gestures to have an increase in responsiveness.

FIG. 15A illustrates a tablet system 2000 having a port 2080 to receive a card 2082 having a SIM circuit 2084 mounted thereon.

FIG. 16 illustrates a preferred cart system for a modular ultrasound imaging system in accordance with the invention. The cart system 2100 uses a base assembly 2122 including a docking bay that receives the tablet. The cart configuration 2100 is configured to dock tablet 2104, including a touch screen display 2102, to a cart 2108, which can include a full operator console 2124. After the tablet 2104, is docked to the cart stand 2108, the system forms a full feature roll about system. The full feature roll about system may include, an adjustable height device 2106, a gel holder 2110, and a storage bin 2114, a plurality of wheels 2116, a hot probe holder 2120, and the operator console 2124. The control devices may include a keyboard 2112 on the operator console 2124 that may also have other peripherals added such as a printer or a video interface or other control devices.

FIG. 17 illustrate a preferred cart system, for use in embodiments with a modular ultrasound imaging system in accordance with the invention. The cart system 2200 may be configured with a vertical support member 2212, coupled to a horizontal support member 2028. An auxiliary device connector 2018, having a position for auxiliary device attachment 2014, may be configured to connect to the vertical support member 2212. A 3 port Probe MUX connection device 2016 may also be configured to connect to the tablet. A storage bin 2224 can be configured to attach by a storage bin attachment mechanism 2222, to vertical support member 2212. The cart system may also include a cord management system 2226, configured to attach to the vertical support member. The cart assembly 2200 includes the support beam 2212 mounted on a base 2228 having wheels 2232 and a battery 2230 that provides power for extended operation of the tablet. The assembly can also include an accessory holder 2224 mounted with height adjustment device 2226. Holders 2210, 2218 can be mounted on beam 2212 or on console panel 2214. The multiport probe multiplex device 2216 connects to the tablet to provide simultaneous connection of several transducer probes which the user can select in sequence with the displayed virtual switch. A moving touch gesture, such as a three finger flick on the displayed image or touching of a displayed virtual button or icon can switch between connected probes.

FIG. 18A illustrates preferred cart mount system for a modular ultrasound imaging system in accordance with the invention. Arrangement 2300 depicts the tablet 2302, coupled to the docking station 2304. The docking station 2304 is affixed to the attachment mechanism 2306. The attachment mechanism 2306 may include a hinged member 2308, allowing for the user display to tilted into a user desired position. The attachment mechanism 2306 is attached to the vertical member 2312. A tablet 2302 as described herein can be mounted on the base docking unit 2304 which is mounted to a mount assembly 2306 on top of beam 2212. The base unit 2304 includes cradle 2310, electrical connectors 2305 and a port 2307 to connect to the system 2302 to battery 2230 and multiplexor device 2216.

FIG. 18B illustrated a card mounted system in which a SIM card 2084 is inserted into unit 2304.

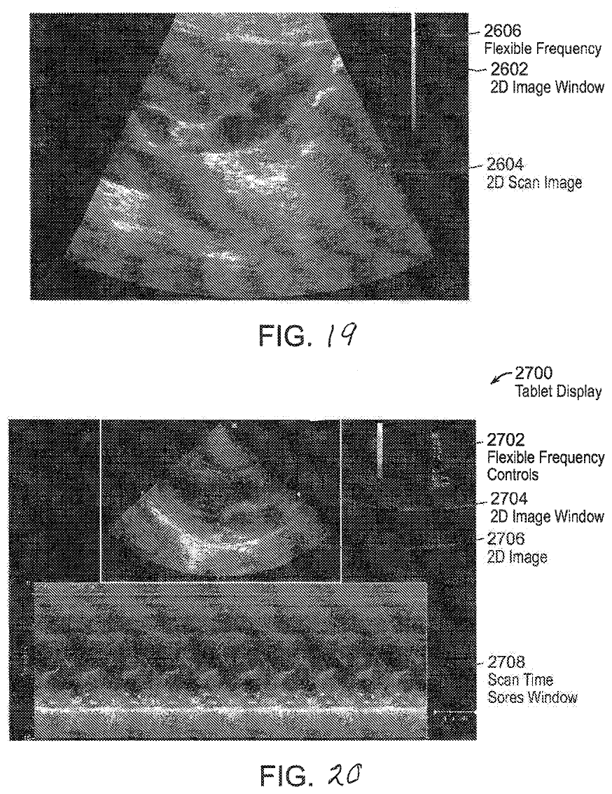

FIG. 19 illustrates a 2D imaging mode of operation with a modular ultrasound imaging system in accordance with the invention. The touch screen of tablet 2504 may display images obtained by 2-dimensional transducer probe using a 256 digital beamformer channels. The 2-dimensional image window 2602 depicts a 2-dimensional image scan 2604. The 2-dimensional image may be obtained using flexible frequency scans 2606, wherein the control parameters are represented on the tablet.

FIG. 20 illustrates a motion mode of operation with a modular ultrasound imaging system in accordance with the invention. The touch screen display of tablet 2700, may display images obtained by a motion mode of operation. The touch screen display of tablet 2700, may simultaneously display 2-dimensional image 2706, and motion mode imaging 2708. The touch screen display of tablet 2700, may display a 2-dimensional image window 2704, with a 2-dimensional image 2706. Flexible frequency controls 2702 displayed with the graphical user interface can be used to adjust the frequency from 2 MHz to 12 MHz.

FIG. 21 illustrates a color Doppler mode of operation with a modular ultrasound imaging system in accordance with the invention. The touch screen display of tablet 2800 displays images obtained by color Doppler mode of operation. A 2-dimensional image window 2806 is used as the base display. The color coded information 2808, is overlaid on the 2-dimensional image 2810. Ultrasound-based imaging of red blood cells are derived from the received echo of the transmitted signal. The primary characteristics of the echo signal are the frequency and the amplitude. Amplitude depends on the amount of moving blood within the volume sampled by the ultrasound beam. A high frame rate or high resolution can be adjusted with the display to control the quality of the scan. Higher frequencies may be generated by rapid flow and can be displayed in lighter colors, while lower frequencies are displayed in darker colors. Flexible frequency controls 2804, and color Doppler scan information 2802, may be displayed on the tablet display 2800.

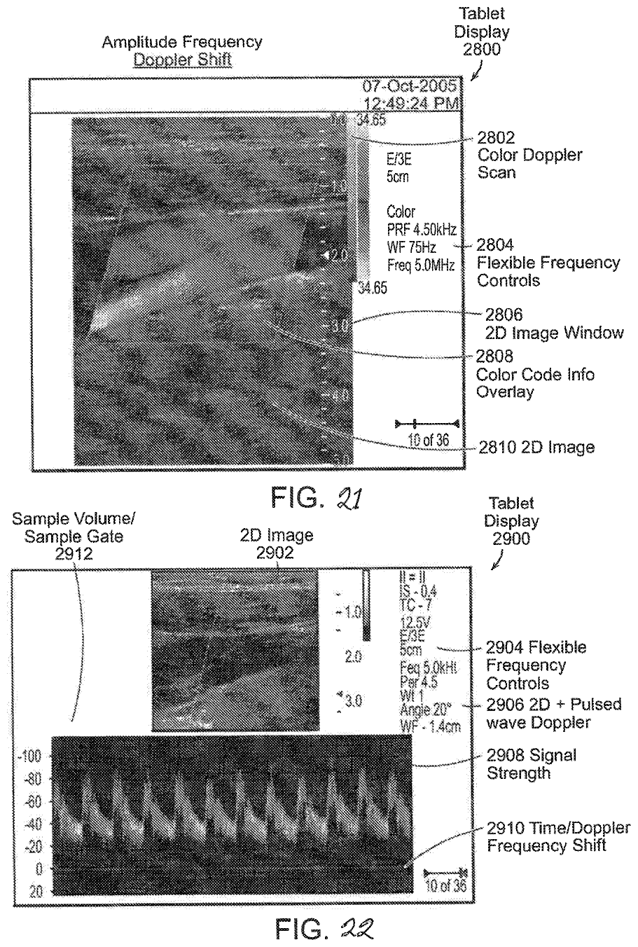

FIG. 22 illustrates a Pulsed wave Doppler mode of operation with a modular ultrasound imaging system in accordance with the invention. The touch screen display of tablet 2900, may display images obtained by pulsed wave Doppler mode of operation. Pulsed wave Doppler scans produce a series of pulses used to analyse the motion of blood flow in a small region along a desired ultrasound cursor called the sample volume or sample gate 2912. The tablet display 2900 may depict a 2-dimensional image 2902, wherein the sample volume/sample gate 2012 is overlaid. The tablet display 2900 may use a mixed mode of operation 2906, to depict a 2-dimensional image 2902, and a time/doppler frequency shift 2910. The time/doppler frequency shift 2910 can be converted into velocity and flow if an appropriate angle between the beam and blood flow is known. Shades of gray 2908, in the time/doppler frequency shift 2910, may represent the strength of signal. The thickness of the spectral signal may be indicative of laminar or turbulent flow. The tablet display 2900 can depict adjustable frequency controls 2904.

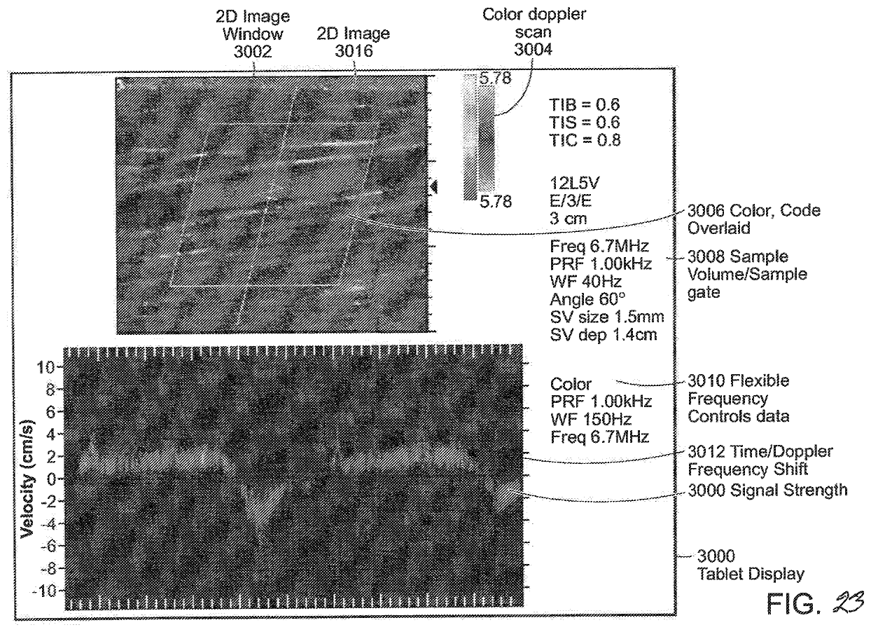

FIG. 23 illustrates a triplex scan mode of operation with a modular ultrasound imaging system in accordance with the invention. The tablet display 3000 may include a 2-dimensional window 3002, capable of displaying 2-dimensional images alone or in combination with the color Doppler or directional Doppler features. The touch screen display of tablet 3000, may display images obtained by color Doppler mode of operation. A 2-dimensional image window 3002 is used as the base display. The color coded information 3004, is overlaid 3006, on the 2-dimensional image 3016. The pulsed wave Doppler feature may be used alone or in combination with 2-dimensional imaging or the color Doppler imaging. The tablet display 3000 may include a pulsed wave Doppler scan represented by a sample volume/sample gate 3008, overlaid over 2 dimensional images 3016, or the color code overlaid 3006, either alone or in combination. The tablet display 3000 may depict a split screen representing the time/doppler frequency shift 3012. The time/doppler frequency shift 3012 can be converted into velocity and flow if an appropriate angle between the insolating beam and blood flow is known. Shades of gray 3014, in the time/doppler frequency shift 3012, may represent the strength of signal. The thickness of the spectral signal may be indicative of laminar or turbulent flow. The tablet display 3000 also may depict flexible frequency controls 3010.

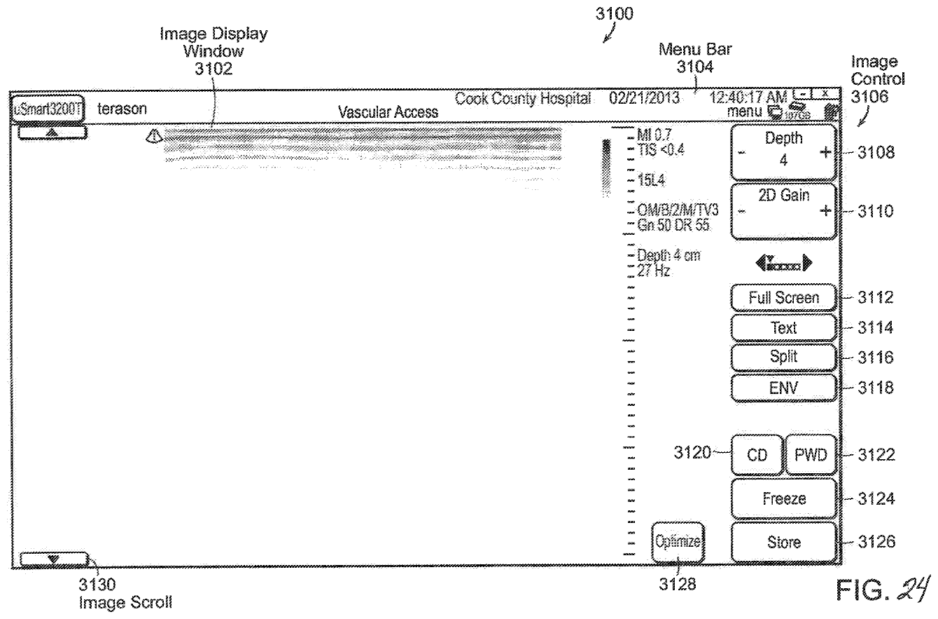

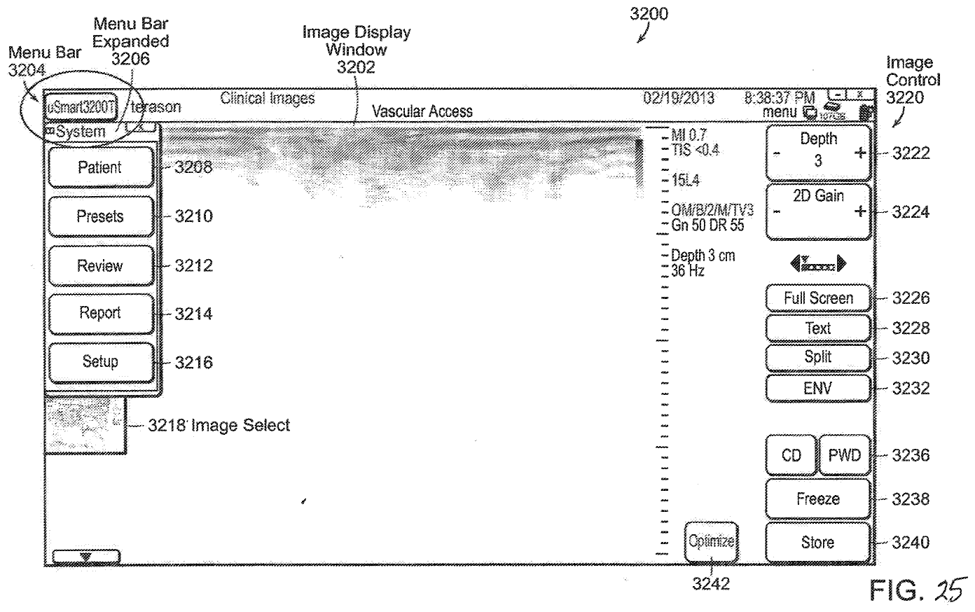

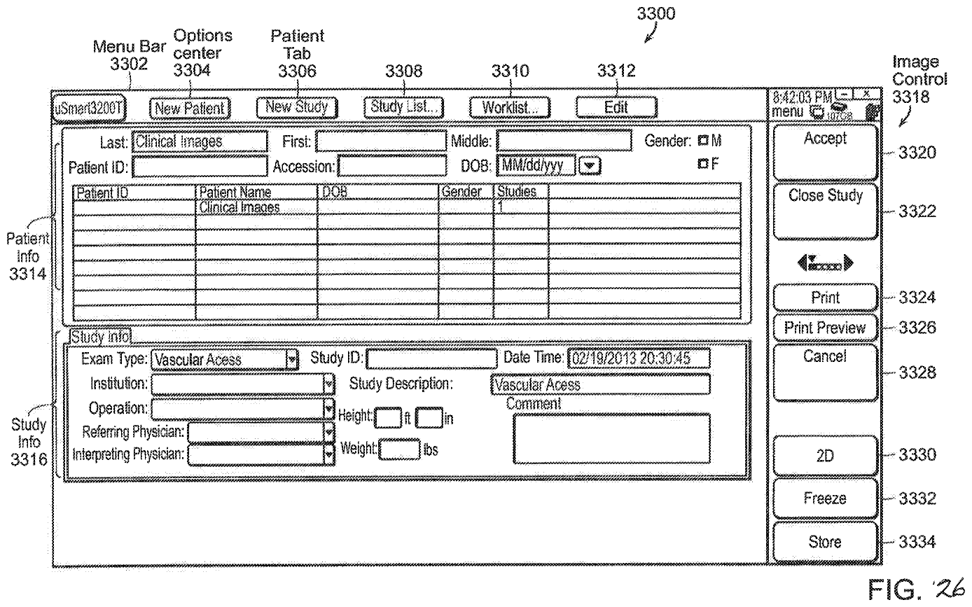

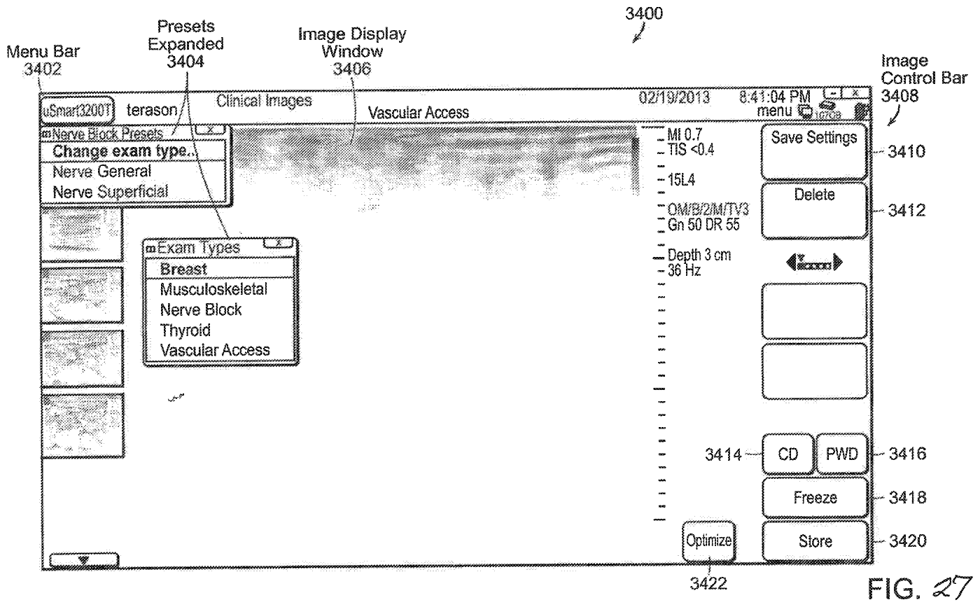

FIG. 24 illustrates a GUI home screen interface 3100, for a user mode of operation, with a modular ultrasound imaging system in accordance with the invention. The screen interface for a user mode of operation 3100 may be displayed when the ultrasound system is started. To assist a user in navigating the GUI home screen 3100, the home screen may be considered as including three exemplary work areas: a menu bar 3104, an image display window 3102, and an image control bar 3106. Additional GUI components may be provided on the main GUI home screen 3100, to enable a user to close, resize and exit the GUI home screen and/or windows in the GUI home screen.

The menu bar 3104 enables users to select ultrasound data, images and/or video for display in the image display window 3102. The menu bar may include components for selecting one or more files in a patient folder directory and an image folder directory.

The image control bar 3106 includes touch controls that may be operated by touch and touch gestures applied by the user directly to the surface of the display. Exemplary touch controls may include, but are not limited to a depth control touch controls 3108, a 2-dimensional gain touch control 3110, a full screen touch control 3112, a text touch control 3114, a split screen touch control 3116, a ENV touch control 3118, a CD touch control 3120, a PWD touch control 3122, a freeze touch control 3124, a store touch control 3126, and a optimize touch control 3128.

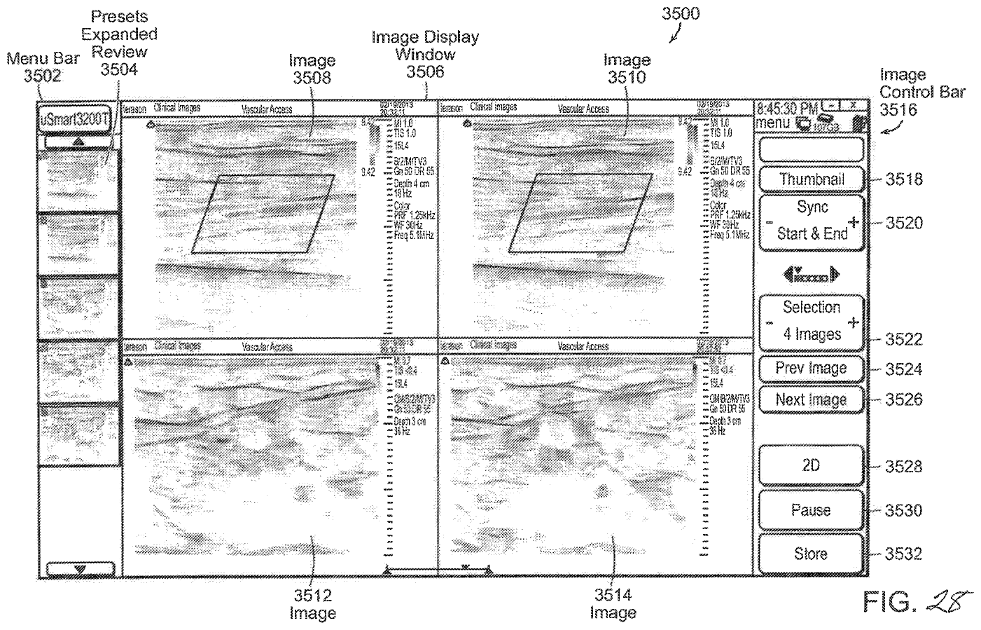

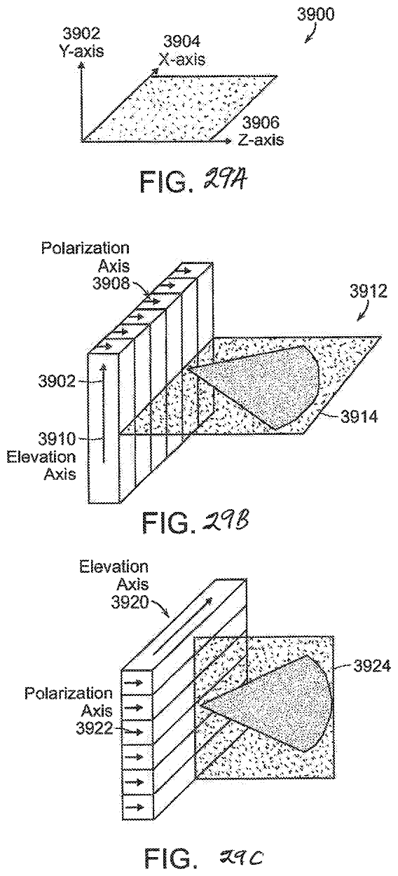

FIG. 25 illustrates a GUI menu screen interface 3200, for a user mode of operation, with a modular ultrasound imaging system in accordance with the invention. The screen interface for a user mode of operation 3200 may be displayed when the menu selection mode is triggered from the menu bar 3204 thereby initiating operation of the ultrasound system. To assist a user in navigating the GUI home screen 3100, the home screen may be considered as including three exemplary work areas: a menu bar 3204, an image display window 3202, and an image control bar 3220. Additional GUI components may be provided on the main GUI menu screen 3200 to enable a user to close, resize, scroll images 3130, and exit the GUI menu screen and/or windows in the GUI menu screen, for example.

The menu bar 3204 enables users to select ultra sound data, images 3218 and/or video for display in the image display window 3202. The menu bar 3204 may include touch control components for selecting one or more files in a patient folder directory and an image folder directory. Depicted in an expanded format 3206, the menu bar may include exemplary touch control such as, a patient touch control 3208, a pre-sets touch control 3210, a review touch control 3212, a report touch control 3214, and a setup touch control 3216.

The image control bar 3220 includes touch controls that may be operated by touch and touch gestures applied by the user directly to the surface of the display. Exemplary touch controls may include, but are not limited to depth control touch controls 3222, a 2-dimensional gain touch control 3224, a full screen touch control 3226, a text touch control 3228, a split screen touch control 3230, a needle visualization ENV touch control 3232, a CD touch control 3234, a PWD touch control 3236, a freeze touch control 3238, a store touch control 3240, and a optimize touch control 3242.

FIG. 26 illustrates a GUI patient data screen interface 3300, for a user mode of operation, with a modular ultrasound imaging system in accordance with the invention. The screen interface for a user mode of operation 3300, may be displayed when the patient selection mode is triggered from the menu bar 3302, when the ultrasound system is started. To assist a user in navigating the GUI patient data screen 3300, the patient data screen may be considered as including five exemplary work areas: a new patient touch screen control 3304, a new study touch screen control 3306, a study list touch screen control 3308, a work list touch screen control 3310, and an edit touch screen control 3312. Within each touch screen control, further information entry fields are available 3314, 3316. For example, patient information section 3314, and study information section 3316, may be used to record data.

Within the patient data screen 3300, the image control bar 3318, includes touch controls that may be operated by touch and touch gestures applied by the user directly to the surface of the display. Exemplary touch controls may include, but are not limited to accept study touch control 3320, close study touch control 3322, print touch control 3324, print preview touch control 3326, cancel touch control 3328, a 2-dimensional touch control 3330, freeze touch control 3332, and a store touch control 3334.

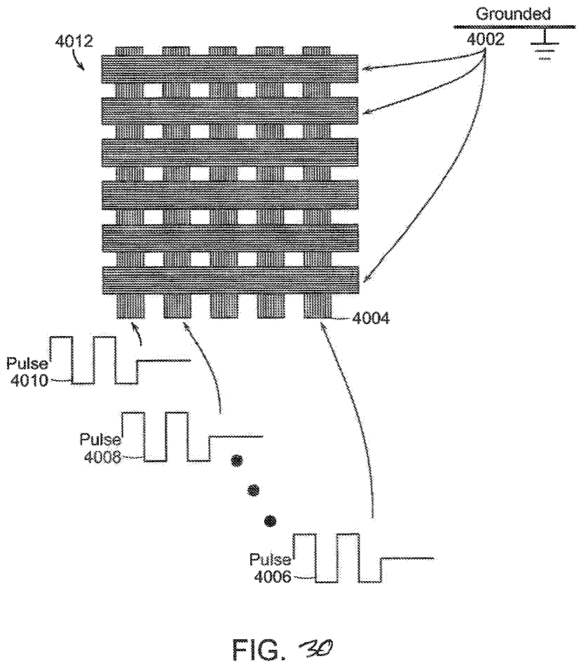

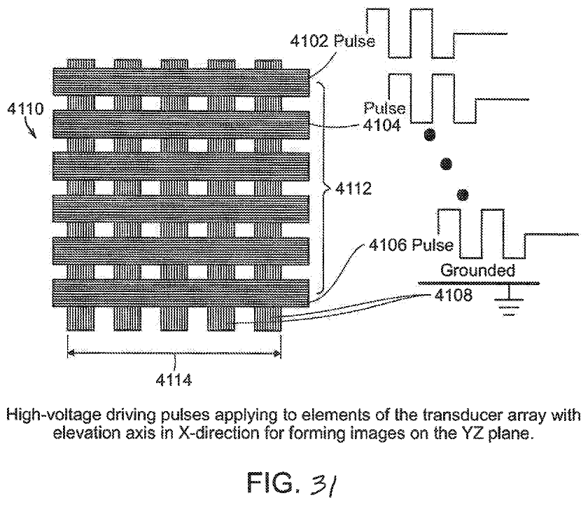

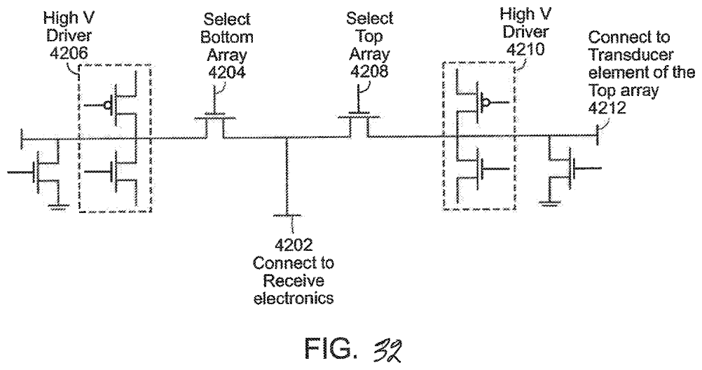

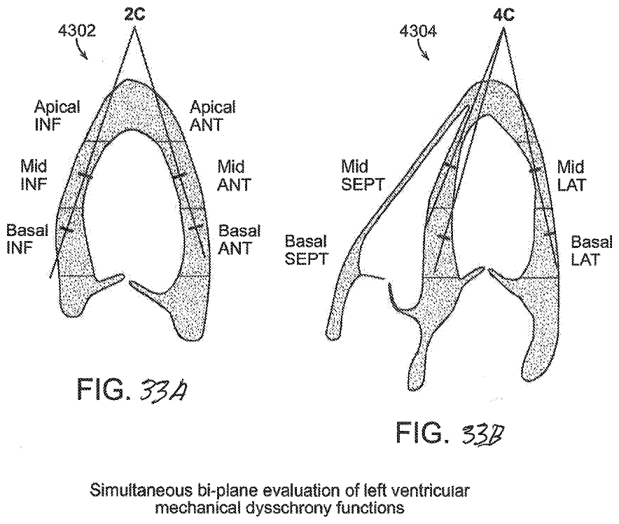

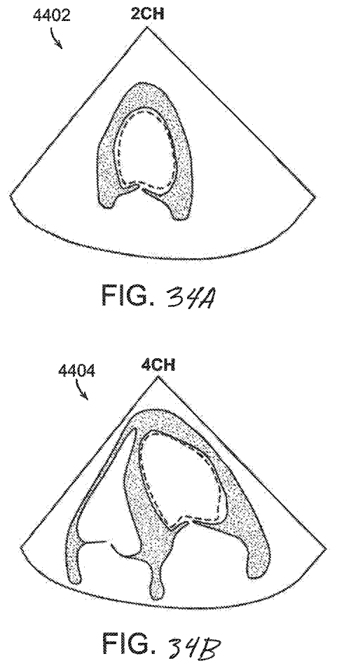

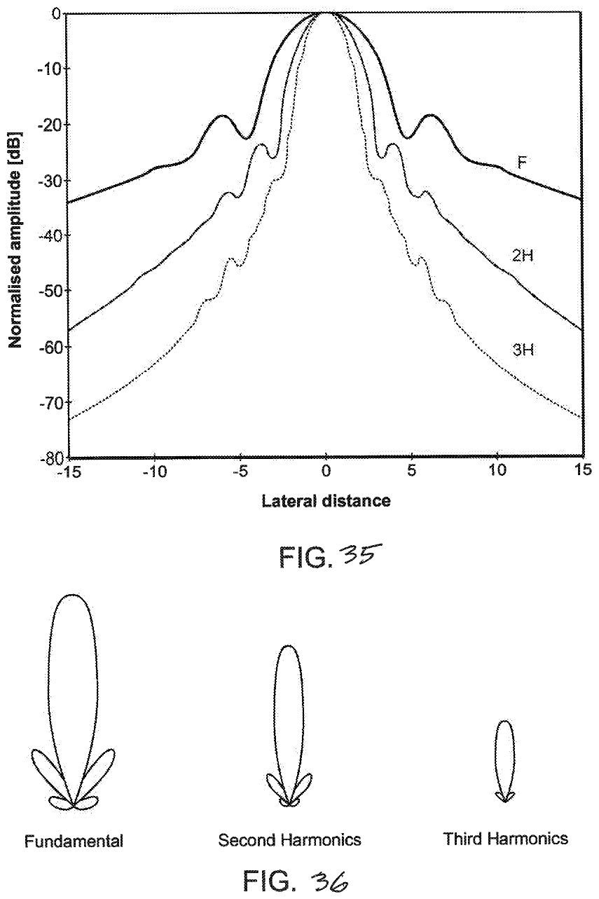

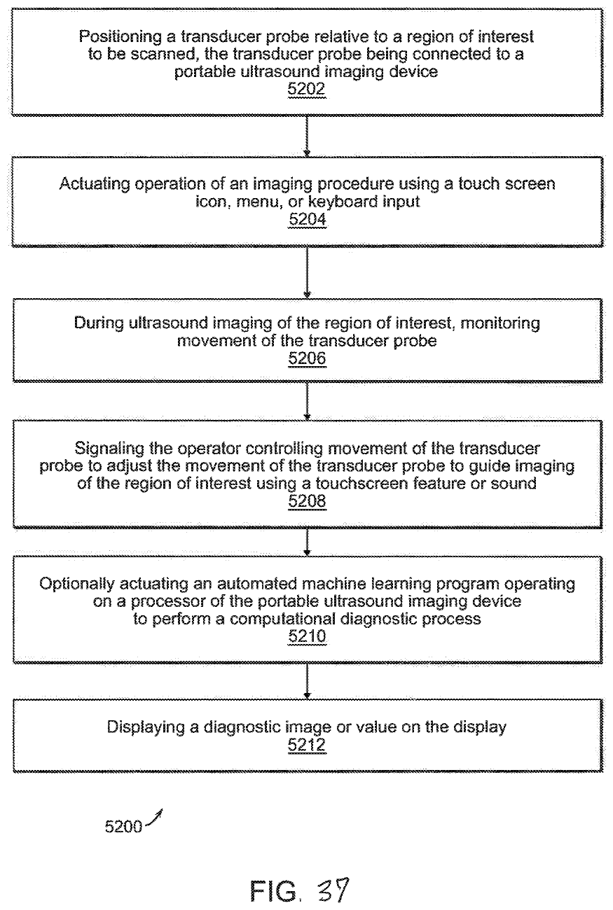

FIG. 27 illustrates a GUI patient data screen interface 3400, for a user mode of operation with a modular ultrasound imaging system in accordance with the invention. The screen interface for a user mode of operation 3400, may be displayed when the pre-sets selection mode 3404, is triggered from the menu bar 3402, when the ultrasound system is started.

Within the pre-sets screen 3400, the image control bar 3408, includes touch controls that may be operated by touch and touch gestures applied by the user directly to the surface of the display. Exemplary touch controls may include, but are not limited to a save settings touch control 3410, a delete touch control 3412, CD touch control 3414, PWD touch control 3416, a freeze touch control 3418, a store touch control 3420, and a optimize touch control 3422.

FIG. 28 illustrates a GUI review screen interface 3500, for a user mode of operation, with a modular ultrasound imaging system in accordance with the invention. The screen interface for a user mode of operation 3500, may be displayed when the pre-sets expanded review 3504, selection mode 3404, is triggered from the menu bar 3502, when the ultrasound system is started.

Within the review screen 3500, the image control bar 3516, includes touch controls that may be operated by touch and touch gestures applied by the user directly to the surface of the display. Exemplary touch controls may include, but are not limited to a thumbnail settings touch control 3518, sync touch control 3520, selection touch control 3522, a previous image touch control 3524, a next image touch control 3526, a 2-dimensional image touch control 3528, a pause image touch control 3530, and a store image touch control 3532.

A image display window 3506, may allow the user to review images in a plurality of formats. Image display window 3506, may allow a user to view images 3508, 3510, 3512, 3514, in combination or subset or allow any image 3508, 3510, 3512, 3514, to be viewed individually. The image display window 3506, may be configured to display up to four images 3508, 3510, 3512, 3514, to be viewed simultaneously.

FIGS. 29A and 29B illustrate an XY bi-plane probe consisting of two one dimensional, multi-element arrays. The arrays may be constructed where one array is on top of the other with a polarization axis of each array being aligned in the same direction. The elevation axis of the two arrays can be at a right angle or orthogonal to one another. Exemplary embodiments can employ transducer assemblies such as those described in U.S. Pat. No. 7,066,887, the entire contents of which is incorporated herein by reference, or transducers sold by Vernon of Tours Cedex, France, for example. Illustrated by FIG. 29A, the array orientation is represented by arrangement 3900. The polarization axis 3908, of both arrays are pointed in the z-axis 3906. The elevation axis of the bottom array, is pointed in y-direction 3902, and the elevation axis of the top array, is in the x-direction 3904.

Further illustrated by FIG. 29B, a one dimensional multi-element array forms an image as depicted in arrangement 3912. A one-dimensional array with an elevation axis 3910, in a y-direction 3902, forms the ultrasound image 3914, on the x-axis 3904, z-axis 3906, plane. A one-dimensional array with the elevation axis 3910, in the x-direction 3904, forms the ultrasound image 3914, on the y-axis 3902, z-axis 3906. A one dimensional transducer array with elevation axis 3910, along a y-axis 3902, and polarization axis 3908, along a z-axis 3906, will result in a ultrasound image 3914, formed along the x 3904 and the z 3906 plane. An alternate embodiment illustrated by FIG. 29C depicts a one-dimensional transducer array with an elevation axis 3920, in a x-axis 904, and a polarization axis 3922, in the z-axis 3906, direction. The ultrasound image 3924, is formed on they 3902 and the z 3906 plane.

FIG. 30 illustrates the operation of a bi-plane image forming xy-probe where array 4012 has a high voltage applied for forming images. High voltage driving pulses 4006, 4008, 4010, may be applied to the bottom array 4004, with a y-axis elevation. This application may result in generation of transmission pulses for forming the received image on the XZ plane, while keeping the elements of the top array 4002 at a grounded level. Such probes enable a 3D imaging mode using simpler electronics than a full 2D transducer array. A touchscreen activated user interface as described herein can employ screen icons and gestures to actuate 3D imaging operations. Such imaging operations can be augmented by software running on the tablet data processor that processes the image data into 3D ultrasound images. This image processing software can employ filtering smoothing and/or interpolation operations known in the art. Beamsteering can also be used to enable 3D imaging operations. A preferred embodiment uses a plurality of 1D sub-array transducers arranged for bi plane imaging.

FIG. 31 illustrates the operation of a bi-plane image forming xy-probe. FIG. 31 illustrates a array 4110, that has a high voltage applied to it for forming images. High voltage pulses 4102, 4104, 4106, may be applied to the top array 4112, with elevation in the x-axis, generating transmission pulses for forming the received image on the yz-plane, while keeping the elements of the bottom array 4108 grounded. This embodiment can also utilize orthogonal 1D transducer arrays operated using sub-array beamforming as described herein.

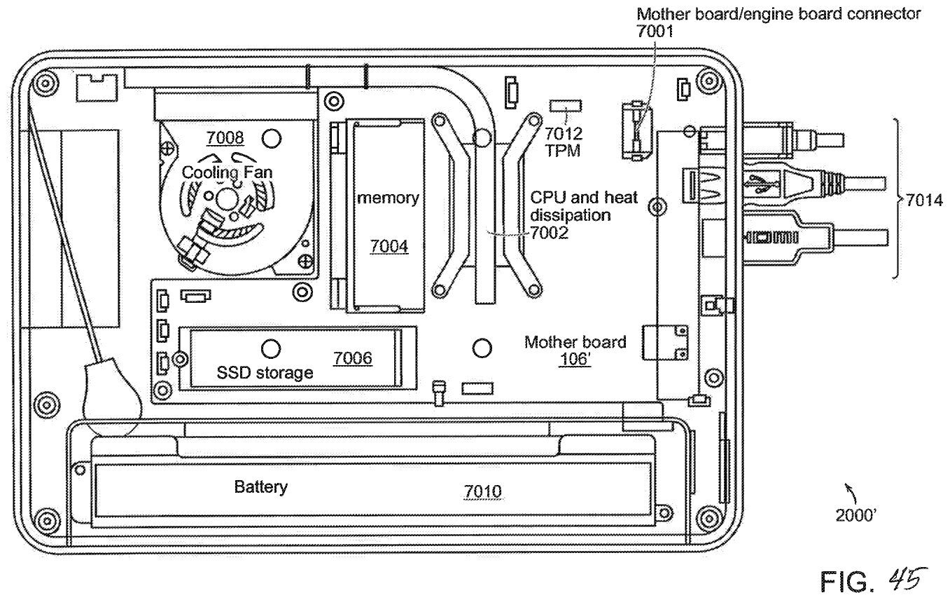

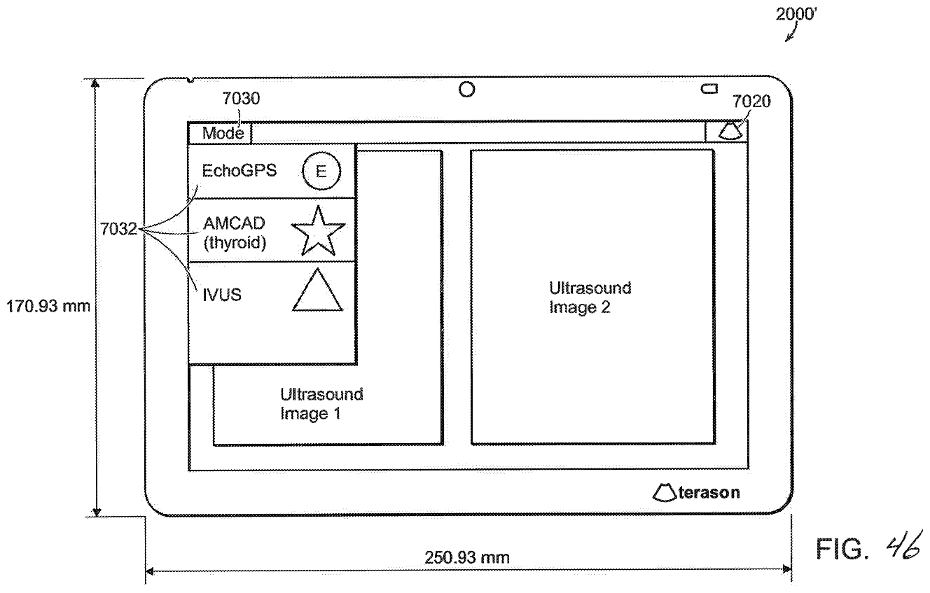

FIG. 32 illustrates the circuit requirements of a bi-plane image forming xy-probe. The receive beamforming requirements are depicted for a bi-plane probe. A connection to receive the electronics 4202, is made. Then elements from the select bottom array 4204, and select top array 4208, are connected to share one connect to the receive electronics 4202 channel. A two to one mux circuit can be integrated on the high voltage driver 4206, 4210. The two to one multiplexor circuit can be integrated into high voltage driver 4206, 4212. One receive beam is formed for each transmit beam. The bi-plane system requires a total of 256 transmit beams for which 128 transmit beams are used for forming a XZ-plane image and the other 128 transmit beams are used for forming a YZ-plane image. A multiple-received beam forming technique can be used to improve the frame rate. An ultrasound system with dual received beam capabilities for each transmit beam provides a system in which two received beams can be formed. The bi-plane probe only needs a total of 128 transmit beams for forming the two orthogonal plane images, in which 64 transmit beams are used to form a XZ-plane image with the other 64 transmit beams for the YZ-plane image. Similarly, for an ultrasound system with a quad or 4 receive beam capability, the probe requires 64 transmit beams to form two orthogonal-plane images. FIGS. 33A-33B illustrate an application for simultaneous bi-plane evaluation. The ability to measure the LV mechanical dyssynchrony with echocardiograph can help identify patients that are more likely to benefit from Cardiac Resynchronization Therapy. LV parameters needed to be quantified are Ts-(lateral-septal), Ts-SD, Ts-peak, etc. The Ts-(lateral-septal) can be measured on a 2D apical 4-chamber view Echo image, while the Ts-SD, Ts-peak (medial), Ts-onset (medial), Ts-peak (basal), Ts-onset (basal) can be obtained on two separated parasternal short-axis views with 6 segments at the level of mitral valve and at the papillary muscle level, respectively, providing a total of 12 segments. FIG. 43A-43B depicts an xy-probe providing apical four chamber 4304, and apical two chamber 4302 images, to be viewed simultaneously.

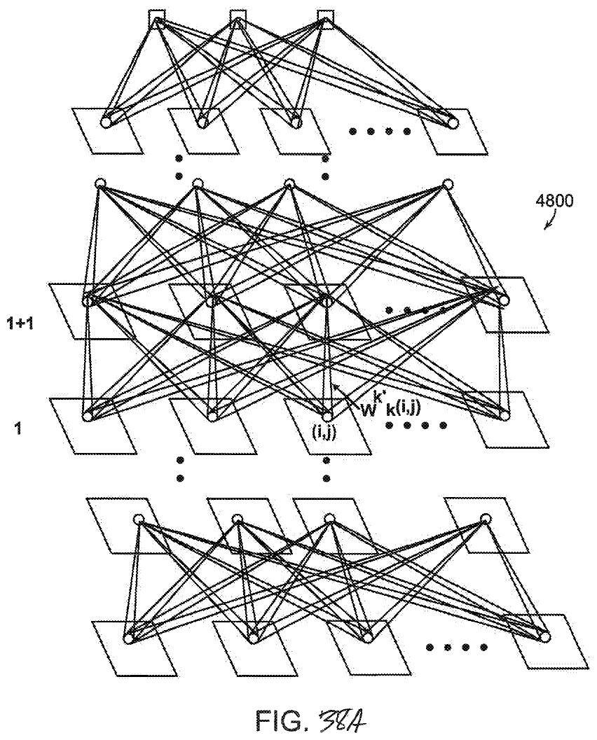

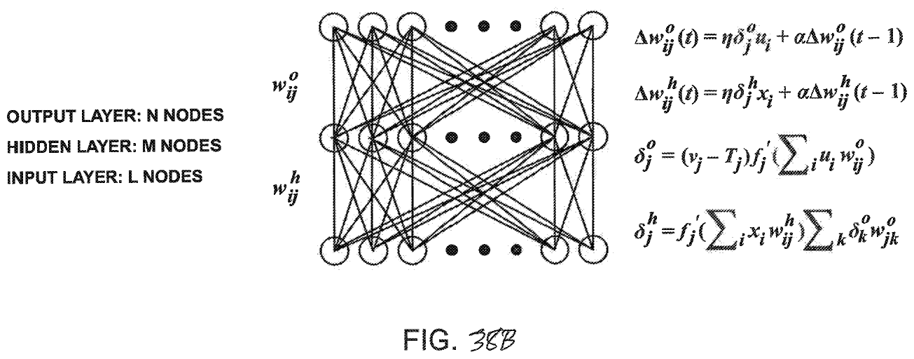

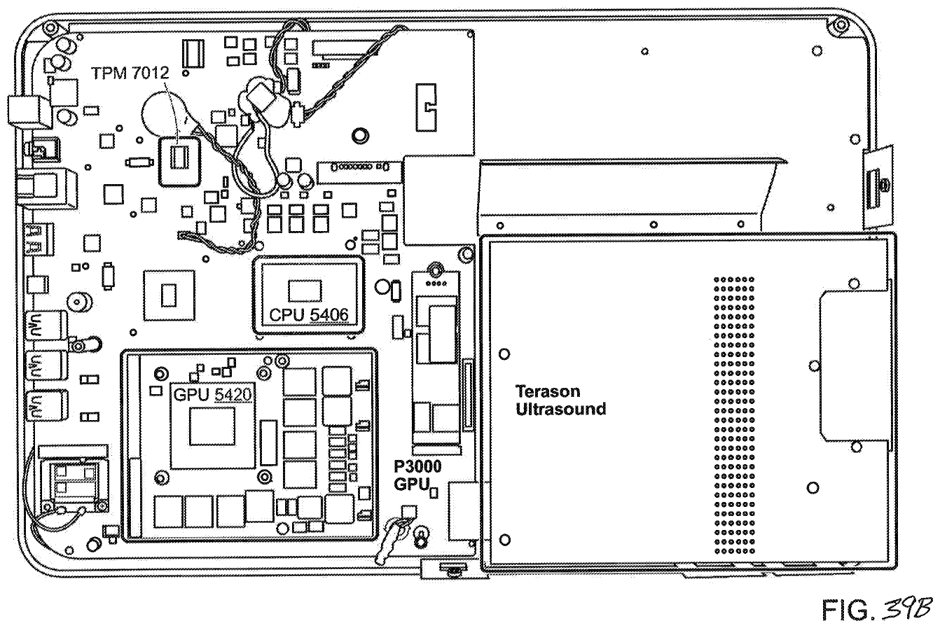

FIGS. 34A-34B illustrate ejection fraction probe measurement techniques. The biplane-probe provides for EF measurement, as visualization of two orthogonal planes ensure on-axis views are obtained. Auto-border detection algorithm, provides quantitative Echo results to select implant responders and guide the AV delay parameter setting. As depicted in FIG. 34A XY probe acquires real-time simultaneous images from two orthogonal planes and the images 4402, 4404 are displayed on a split screen. A manual contour tracing or automatic border tracing technique can be used to trace the endocardial border at both end-systole and end-diastolic time from which the EF is calculated. The LV areas in the apical 2CH 4402, and 4CH 4404, views, A1 and A2 respectively, are measured at the end of diastole and the end of systole. The LVEDV, left ventricular end-diastolic volume, and LVESV, left ventricular the end-systole volume, are calculated using the formula V=8/3πA₁A₂/L. And the ejection fraction is calculated by EF=LVEDV−LVESD/LVEDV.

In the medical ultrasound industry, almost every ultrasound system can do harmonic imaging, but this is all done by using 2nd harmonics or fₒ, where fₒ is the fundamental frequency. Preferred embodiment of the present invention use higher order harmonics, i.e., 3fₒ, 4fₒ, 5fₒ etc. for ultrasound imaging. Harmonics higher than the 2nd order, provide image quality and spatial resolution that are substantially improved. The advantages of higher order harmonics include improving spatial resolution, minimizing clutter and providing image quality with clear contrast between different tissue structures and clearer edge definition. This technique is based on the generation of harmonic frequencies as an ultrasound wave propagates through tissue. The generation of harmonic frequencies is related to wave attenuation due to nonlinear sound propagation in tissue that results in development of harmonic frequencies that were not present in the transmitted wave. The requirements for achieving this superharmonic imaging are 1) low-noise wideband width linear amplifier; 2) high-voltage, linear transmitter; 3) wide bandwidth transduce; and 4) advanced signal processing.

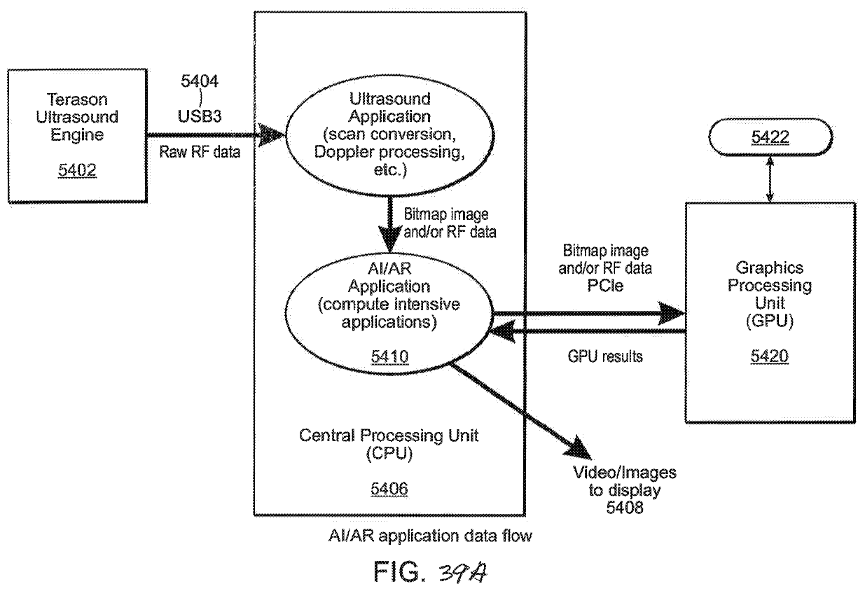

FIG. 37 illustrates an imaging sequence 5200 using position tracking of a transducer probe. The sequence 5200 includes positioning a transducer probe relative to a region of interest to be scanned, the transducer probe being connected to a portable ultrasound imaging device (step S202). The sequence 5200 includes actuating operation of an imaging procedure using a touch screen icon, menu, or keyboard input (step S204). The sequence 5200 includes monitoring movement of the transducer probe during ultrasound imaging of the region of interest (step S206). The sequence 5200 includes signaling the operator controlling movement of the transducer probe to adjust the movement of the transducer probe to guide imaging of the region of interest using a touchscreen feature or sound (step S208). The sequence 5200 optionally includes actuating an automated machine learning program operating on a processor of the portable ultrasound imaging device to perform a computational diagnostic process (step S210). The sequence 5200 includes displaying a diagnostic image or value on the display (step S212).

Artificial intelligence (AI) and Augmented reality (AR) are transforming the medical ultrasound. Medical ultrasound applications using AI and AR can solve critical problems impacting patient outcomes in many diagnostic and therapeutic applications. Ultrasound imaging poses problems that are solved with deep learning because it takes years of training to learn how to read ultrasound images. Clinical studies based on deep learning AI algorithms for automatically detecting the tumor regions and for detecting heart disease to assist medical diagnosis with high sensitivity and specificity have been reported. Augmented reality (AR) fuses optical vision video with ultrasound images providing real-time image guidance to surgeons for improved identification of anatomical structure and enhanced visualization during surgical procedures. Ultrasound system used for image acquisition can employ computer systems with more than 1000GFLOPs (giga floating point operations per second) of processing power to carry out the mathematical computation imposed by the deep learning algorithm, or the computation required for fusing/superimposing an ultrasound image on a user's optical view of an anatomical feature. AI and/or AR can drastically enhance or expand ultrasound imaging applications. A computational enhanced ultrasound system that can acquire real-time ultrasound images and also can carry out the large amount of computations mandated by those algorithms can advance clinical care delivery in cancer treatment and in cancer and heart disease diagnosis. The integration of improvements in portability, reliability, rapidity, ease of use, and affordability of ultrasound systems along with computational capacity for advanced imaging are provided in preferred embodiments herewith.

Ultrasound (US) images have been widely used in the diagnosis and detection of cancer and heart disease, etc. The drawback of applying these diagnostic techniques for cancer detection is the large time consumed in the manual diagnosis of each image pattern by a trained radiologist. While experienced doctors may locate the tumor regions in a US image manually, it is highly desirable to employ algorithms that automatically detect the tumor regions in order to assist medical diagnosis. Automated classifiers substantially upgrade the diagnostic process, in terms of both accuracy and time requirement by distinguishing benign and malignant patterns automatically. Neural networks (NN) play an important role in this respect, especially in the application of breast and prostate cancer detection, for example.

Pulse-coupled neural networks (PCNNs) are a biologically inspired type of neural network. It is a simplified model of a cat's visual cortex with local connections to other neurons. PCNN has the ability to extract edges, segments, and texture information from images. Only a few changes to the PCNN parameters are necessary for effective operation on different types of data. This is an advantage over published image processing algorithms that generally require information about the target before they are effective. An accurate boundary detection algorithm of the prostate in ultrasound images can be obtained to assist radiologists in rendering a diagnosis. To increase the contrast of the ultrasound prostate image, the intensity values of the original images are first adjusted using the PCNN with a median filter. This can be followed by the PCNN segmentation algorithm to detect the boundary of the image. Combining intensity adjustment and segmentation enables the reduction of PCNN sensitivity to the settings of the various PCNN parameters whose optimal selection can be difficult and can vary even for the same problem. The results show that the overall boundary detection overlap accuracy offered by the employed PCNN approach is high compared with other machine learning techniques including Fuzzy C-mean and Fuzzy Type-II.

Ultrasound (US) images have been widely used in the diagnosis of breast cancer in particular. While experienced doctors may locate the tumor regions in a US image manually, it is highly desirable to develop algorithms that automatically detect the tumor regions in order to assist medical diagnosis. An algorithm for automatic detection of breast tumors in US images has been developed by Peng Jiang, Jingliang Peng, Guoquan Zhang, Erkang Cheng, Vasileios Megalooikonomou, Haibin Ling; “Learning-based Automatic Breast Tumor detection and Segmentation in Ultrasound Images”, the entire contents of which is incorporated herein by reference. The tumor detection process was formulated as a two-step learning problem: tumor localization by bounding box and exact boundary delineation. Specifically, an exemplary method uses an AdaBoost classifier on Han-like features to detect a preliminary set of tumor regions. The preliminarily detected tumor regions are further screened with a support vector machine (SVM) using quantized intensity features. Finally, the random walk segmentation algorithm is performed on the US image to retrieve the boundary of each detected tumor region. The preferred method has been evaluated on a data set containing 112 breast US images, including histologically confirmed 80 diseased patients and 32 normal patients. The data set contains one image from each patient and the patients are from 31 to 75 years old. These measurements demonstrate that the proposed algorithm can automatically detect breast tumors, with their locations and boundary.

Rheumatic heart disease (RHD) is the most commonly acquired heart disease in young people under the age of 25. It most often begins in childhood as strep throat, and can progress to serious heart damage that kills or debilitates adolescents and young adults, and makes pregnancy hazardous.

Although virtually eliminated in Europe and North America, the disease remains common in Africa, the Middle East, Central and South Asia, the South Pacific, and in impoverished pockets of developed nations. Thirty-three million people around the world are affected by RHD. While RHD can be diagnosed by ultrasound images, such ultrasound images are very user dependent. Typically, it requires very experience sonographer to acquire diagnostic quality ultrasound images. It is beneficial to patients to employ an AI based deep learning algorithm to put ultrasound systems in the hands of general practitioner to diagnose RHD, by training a system with GPU-accelerated deep learning software to provide diagnostic ultrasound images.

A computational neural network model with fully connected artificial neural nodes is shown in FIG. 38A. The model comprises L layers with K nodes within each hidden layer. The output of each node in the lower layer is fully connected to the corresponding node in the upper layer with a trainable connecting weight.

As can be seen in FIG. 36A, each node is a two dimensional image where (i,j) represents pixel element location; N_{l,k}(i,j) represents the (i,j) pixel value in the k^{th} location of the/layer; W_{l,k}^{k′}(i,j) represents the connecting weight between the (i,j)^{th} element of the k^{th} location in the l layer with the (i,j) element in the k′^{th} location of the l+1, upper, layer. The pixel value, N_{l+1,k′}(i,j), at the k′^{th} location of the upper layer can be computed by summing the products of connecting weights, W_{l,k}, to each corresponding nodes at the lower layer and the output values from each of the nodes in the lower, l, layer, N_{l,k}(I,j) for i=1, 2, . . . , I; j=1, 2, . . . , J, i.e.,

$${N}_{1 + 1 , {k}^{′}} ( i , j ) = {{\sum }^{K}}_{k = 1} {W}_{1 , k}^{{k}^{′}} ( i , j ) {N}_{1 , k} ( i , j ) \qquad ( 5 )$$

Assume an image size of (1000, 1000), i.e., i=1000, j=1000, in each of the neural nodes in the hidden layer, and there are 500 nodes, k=500, within each hidden layer in this example. It is straightforward to compute the mathematical operations that need to be carried out to compute the values of the nodes on the upper layer from the inputs from the lower layer, i.e., 1×10⁹ floating point operations. For a neural network with 1000 layers, i.e., 1=1000, the total number of computations required is 1×10¹² floating point operations, i.e., a processor with 1000GFLOPs is needed to compute the required data using this deep learning artificial neural network in carrying out the RHD clinical evaluation in developing countries. In addition to the ultrasound system, clinicians can carry 76 high-end linux laptops with Nvidia GPUs with more than 1000GFLOPs processing power. Preferred embodiments of the present application include a tablet ultrasound system as described herein in which a graphic processing unit is integrated into the tablet or portable system housing and is connected via bus or other high speed/data rate connection to the central processor of the ultrasound system.

A neural network comprises units (neurons), arranged in layers, which convert an input vector into some output. Each unit takes an input, applies a (often nonlinear) function to it and then passes the output on to the next layer. Generally the networks are defined to be feed-forward: a unit feeds its output to all the units on the next layer, but there is no feedback to the previous layer. Weightings are applied to the signals passing from one unit to another, and it is these weightings which are tuned in the training phase to adapt a neural network to the particular problem at hand. This is the learning phase. The goal of neural network pattern recognition is to group observed input patterns into one of a set of known classes. The back-propagation classifier is one of the most intensively studied NN classifiers (NNCs) and has been applied to problems, for example, in face, character and speech recognition and in signal prediction. Radial basis function (RBF) classifiers generalize effectively in high-dimensional spaces and provide low error rates with training times much less than those of backpropagation classifiers. In addition, RBF classifiers form smooth, well-behaved decision regions and perform well with little training data. In the following, the real-time implementation of a back-propagation algorithm and an RBF algorithm are described. In addition, back-propagation and RBF training algorithms are described.

Backpropagation is a method widely used in artificial neural networks in remote sensing image classification to calculate the error contribution of each neuron after a batch of data (in image recognition, multiple images) is processed. In the context of machine learning, backpropagation is commonly used by the gradient descent optimization algorithm to adjust the weight of neurons by calculating the gradient of the loss function. This technique is also sometimes called backward propagation of errors, because the error is calculated at the output and distributed back through the network layers.

Backpropagation requires a known, desired output for each input value. It is therefore considered to be a supervised learning method (although it is used in some unsupervised networks such as autoencoders). Backpropagation is also a generalization of the delta rule to multi-layered feedforward networks, made possible by using the chain rule to iteratively compute gradients for each layer. It is closely related to the Gauss-Newton algorithm, and is part of continuing research in neural backpropagation. Backpropagation can be used with any gradient-based optimizer, such as L-BFGS or truncated Newton.

The back-propagation neural network was developed by Rumelhart et al. as a solution to the problem of training multi-layer perceptrons. Backpropagation is commonly used to train deep neural networks, a term used to describe neural networks with more than one hidden layer. Research has shown that the precision of the image classification has been greatly improved by neural network model for supervised classification of remote sensing images because neural network classifiers can study discontinuous, non-linear classification models. In addition, neural network models have good robustness and self-adaptability and are able to end the question in the specific conditions. Finally, neural networks are able to combine analysis of multiple parameters of the remote sensing image such as shape, spectral, texture and so on to extract the potential information.

The back-propagation training algorithm is an iterative gradient descent method designed to minimize the mean square error between the actual output of a multilayer feed-forward and the desired output. The algorithm starts with a network having random weights. Training vectors are applied repeatedly to the network, and weights are adjusted after each training vector according to a set of equations specified by the algorithm until the weights converge and the error function is reduced to an acceptable value.

The computation algorithm is summarized next. As indicated in FIG. 38B, x, represents the input vector, wᵢⱼ^{h} the connection weights between the input and the hidden layers, and wᵢⱼ^{o} the connection weights between the hidden and the output layers. In addition, uⱼ=f(yⱼ) represents activation from the hidden layer, where yⱼ=xᵢ wᵢⱼ^{h} is the dot-product output, and vⱼ=f(zⱼ)=f(Σᵢ uᵢwᵢⱼ⁰) is the j^{th} element of the actual output pattern produced by the network. In both cases f(.) is the nonlinear activation function of a node. In the weight-update phase, the amount by which the weights wᵢⱼ^{o}(t) and wᵢⱼ^{h}(t) are updated, respectively, are given by

Δwᵢⱼ^{o}(t)=ηδⱼ^{o}uᵢ+αΔwᵢⱼ^{o}(t−1) (6)

and

Δwᵢⱼ^{h}(t)=ηδⱼ^{h}xᵢ+αΔwᵢⱼ^{h}(t−1) (7)

where t is a time index. The delta terms are specified by the following equations:

δⱼ^{o}(vⱼ−Tⱼ)fⱼ′(Σᵢuᵢwᵢⱼ^{o}) (8)

δⱼ^{h}=fⱼ′(Σᵢxᵢwᵢⱼ^{h})Σₖδkₒ^{w}jₖ^{o} (9)

In Eq. (8), Tⱼ is the j^{th} component of the target output pattern. The implementation of the back-propagation training rule thus involves two phases. During the first phase, the input is presented and propagated forward through the network to compute the output values uⱼ and vⱼ. During the second phase, starting at the output node, the error terms are propagating backward to the nodes in the lower layers and the weights are adjusted accordingly.

An RBF classifier has an architecture very similar to that of the three-layer feed-forward net. FIG. 38B shows an RBF classifier where connections between the input and hidden layers have unit weights and, as a result, do not have to be trained. Nodes in the hidden layer, called basis function (BF) nodes, can have a Gaussian pulse nonlinearity specified by a particular mean vector μᵢ and variance vector σᵢ², where i=1, 2, . . . , F and F is the number of BF nodes. Given an N-dimensional input vector X, each BF node i outputs a scalar value y reflecting the activation of the BF caused by the input:

$${y}_{i} = {Φ}_{i} ( ‖ X - {μ}_{i} ‖ ) = exp [ - {\sum }_{k = 1}^{N} \frac{{( {x}_{k} - {μ}_{ik} )}^{2}}{2 h {σ}_{k}^{2}} ] \qquad ( 10 )$$

where h is a proportional constant for the variance, xₖ is the k^{th} component of the input vector X=[x₁, x₂, . . . , x_{N}], and μᵢₖ and σₖ² are the kth components of the mean and variance vectors, respectively, of basis function node i. Inputs that are close to the center of the radial BF (in the Euclidean sense) result in a higher activation, while those that are far away result in low activation. Since each output node of the RBF network forms a linear combination of the BF node activations, the network connecting the middle and output layers is linear:

zⱼ=Σᵢwᵢⱼyᵢ+w₀ⱼ (11)

where zⱼ is the output of the j^{th} output node, yᵢ is the activation of the i^{th} BF node, wᵢⱼ is the weight connecting the i^{th} BF node to the j^{th} output node, and wₒⱼ is the bias or threshold of the j^{th} output node. This bias comes from the weight associated with a BF node (in this case BF node i=0) that has a constant unit output regardless of the input. An unknown input vector X is classified as belonging to the class associated with the output node j with the largest output zⱼ.

It is important to note that in Eq. (10), the RBF (0 is chosen to be a Gaussian function). In general, if the first derivative of a function is completely monotonic, this function can be used as a radial basis function. A list of functions that can be used in practice for classification is given below

$$Φ ( ‖ X - {μ}_{i} ‖ ) = \frac{1}{{( {c}^{2} + {r}^{2} )}^{α}} α > 0 \qquad ( 12 )$$

$$Φ ( ‖ X - {μ}_{i} ‖ ) = {( {c}^{2} + {r}^{2} )}^{β} 0 < β < 1$$

where r≡Σₖ(xₖ−μᵢₖ)².

The weights Wᵢⱼ in the linear network can be trained using an iterative gradient descent method to minimize the mean square error between the actual output of a RBF network and the desired output. To illustrate this approach, let the actual RBF classifier output for a given input vector X with class label C at output node j be zⱼ, and the desired output in a given example be, e.g., 4, where

dⱼ=0,otherwise,j=I, . . . ,M (13)

and M is the number of classes. In Eq. (13), dⱼ is the j^{th} component of the desired target output pattern. Let the optimal weights be defined as those which minimize the square error of the net output:

$$E = \frac{1}{2} {\sum }_{j = 1}^{M} {[ {d}_{j} - {z}_{j} ]}^{2} \qquad ( 14 )$$

The minimum error can be achieved by selecting weight changes in the direction opposite to the gradient of this error function, thus performing a gradient descent of the error function.

### That is:

$$Δ {w}_{i j} = - \frac{\partial E}{\partial {w}_{ij}} = - \frac{\partial E}{\partial {z}_{j}} \frac{\partial {z}_{j}}{\partial {w}_{ij}} \qquad ( 15 )$$

It follows then

Δwᵢⱼ=−(zⱼ−dⱼ)yᵢ (16)

The algorithm starts with a network with random weights. Training vectors are applied repeatedly to the network and weights are adjusted after each training vector according to Eq. (16) until weights converge and the error function is reduced to an acceptable value.

The computation algorithm is summarized next. As indicated in the network structure shown in FIG. 56B, X, represents the input vector, while the wᵢⱼ represents the connection weights between the hidden BF nodes and the output layer. The implementation of the RBF training rule thus involves two phases. During the first phase, the input is presented and propagated forward through the network to compute the output values yᵢ and zⱼ. During the second phase, the weights are adjusted according to Eq. (16). The procedure repeats until weights converge and the error term is reached to an acceptable value.

When the user selects an imaging mode requiring more complex computational or imaging processing functions, processor 5406 will access machine learning and/or image processing applications 5410 described herein as shown in FIG. 39A. This can include the selectable option of processing the RF data generated by the transducer that can also be forwarded from the engine 5402 via bus 5404 to processor 5410. The ultrasound application 5405 can utilize the RF data or data formatted as bitmap image data for processing by processing applications 5410 that transmit the required data to graphics processing unit 5420. Processor 5420 can utilize memory 5422 store data for further processing or for transmission back to central processor applications 5410 prior storage display, or wired/wireless transmission to a network.

FIG. 39B depicts a photograph of a circuit board layout for a tablet configuration wherein the processor 5406 is mounted on a single circuit board with the graphics processing unit 5420. The tablet can have a display diameter in a range of 8-16 inches in which all operations can be actuated by touch operation. Alternatively, the tablet can also display a touch actuated icon for voice activation, can be operated by an external keyboard, or remotely operated via a network by wired or wireless connection.

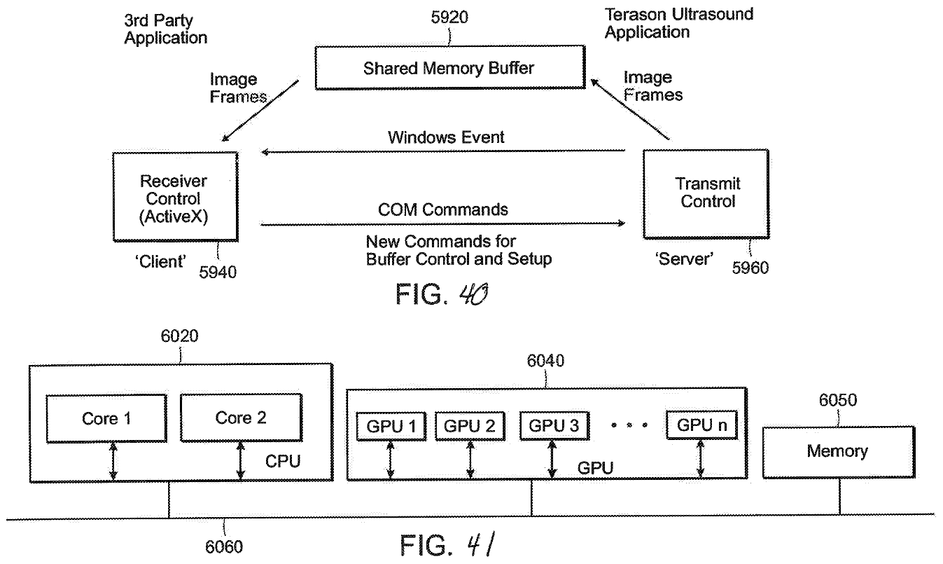

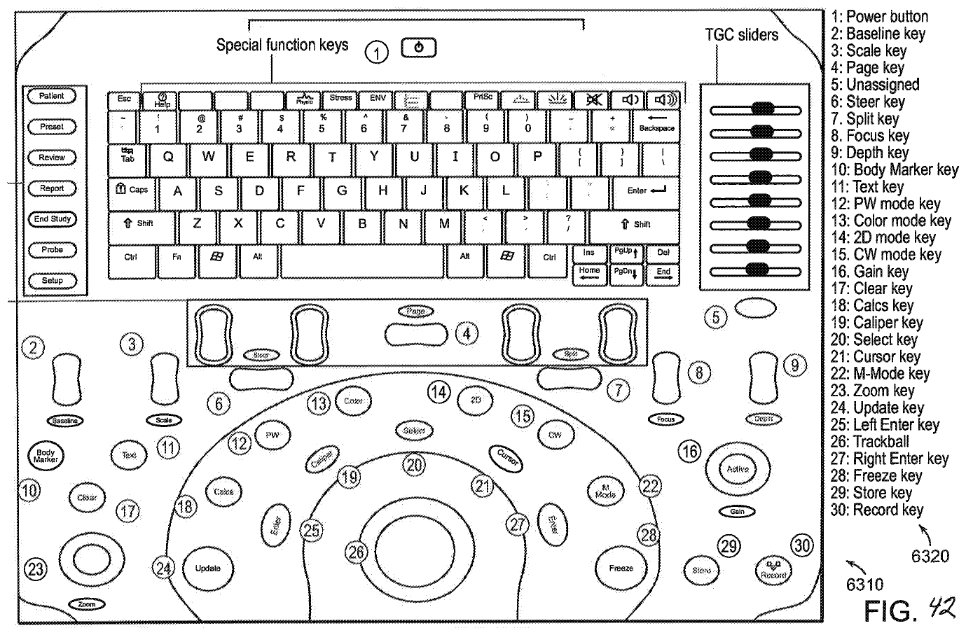

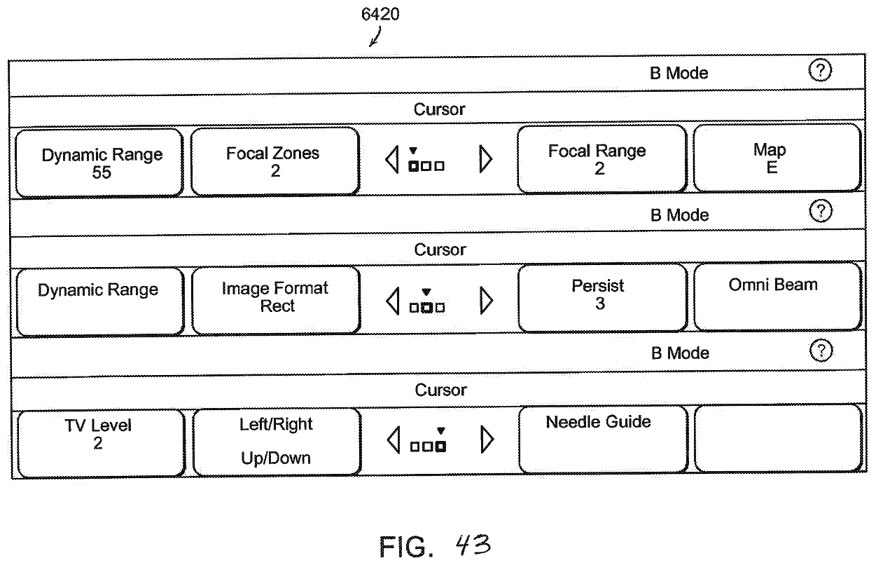

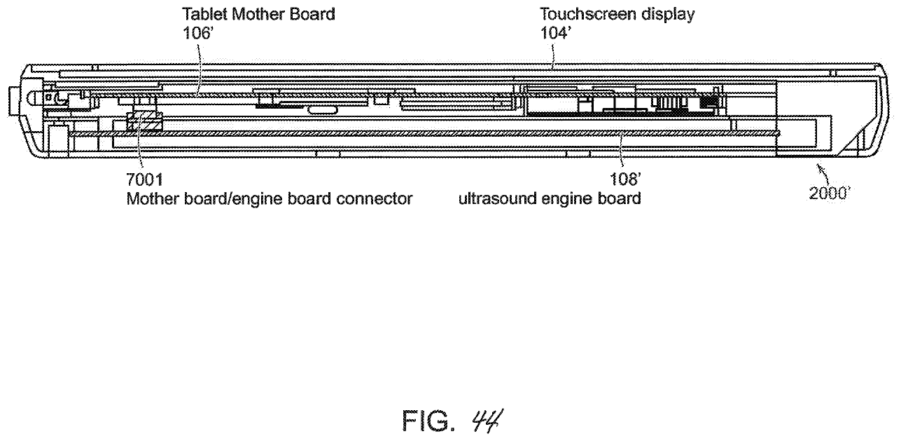

FIG. 40 illustrates the use of a shared memory to provide communication with an external application. In tablet or other portable ultrasound devices utilizing a shared memory as described herein a plurality of different applications on the same or different processors can access the stored data. Further details regarding shared memory operations in ultrasound devices can be found in U.S. Pat. No. 9,402,601 and in U.S. Published Application No 2004/015079, filed on Mar. 11, 2003 the entire contents of this patent and the application is incorporated herein by reference. The shared memory 5920 can be accessed using a control circuit 5960 in the tablet or laptop computer which send and receive packets of data to a third party application 5940 running remotely, or internally in the tablet or portable ultrasound device as described herein. The shared memory 5920 can be used to transmit individual image frames or streaming video for processing using a third party application, which can include machine learning or augmented reality operations. FIG. 41 depicts a distributed processor system or GPU 4954 integrated into a tablet or laptop ultrasound system. A plurality of core processor 6020 can be connected via bus 6060 to a plurality of GPUs 6040 and a shared memory 6050. Tablet devices employing touch screen actuation of the ultrasound imaging operation can include a touch actuated menu of operations performed by the graphics processor. For example, a software program available from Bay Labs, Inc. can be opened by a touch actuated icon or menu list on the tablet touchscreen. An exemplary program used with an imaging procedure can be the EchoMD Auto EF product available from Bay Labs, Inc. to automatically select images or video from an echocardiographic study and also perform automated ejection fraction amputation.

A user can view a saved study in the review window. While reviewing a saved study, a user can add annotations and measurements in the same way as on the Imaging window.

The exemplary portable ultrasound system includes a console shown 6310 in FIG. 42 that houses control 6320 that configure and operate the portable ultrasound system.

1: Power button
2: Baseline key
3: Scale key
4: Page key
5: Unassigned
6: Steer key
7: Split key
8: Focus key
9: Depth key
10: Body Marker key
11: Text key
12: PW mode key
13: Color mode key
14: 2D mode key
15: CW mode key
16: Gain/Active control
17: Clear key
18: Calcs key
19: Caliper key
20: Select key
21: Cursor key
22: M-Mode key
23: Zoom control
24: Update key

The console includes an alphanumeric keyboard, a group of system keys, TGC sliders, softkey controls, and numerous controls for ultrasound imaging functions. The numbered Ultrasound Imaging controls in the exemplary console perform the functions listed below:
1. Power: Starts the system and shuts it down.
2. Baseline: Changes the Doppler baseline in PW, CW and Color Doppler modes. Pressing the top of the key moves the baseline up, and pressing the bottom of the key moves it down.
3. Scale: Changes the velocity scale (by changing the PRF) in PW, CW and Color Doppler modes. Pressing the top of the key increases the PRF, and pressing the bottom of the key decreases it.
4. Page: Changes which set of active softkeys are displayed.
5. This key may be unassigned.
6. Steer: In 2D, Color Doppler or PWD modes, this key steers the ultrasound signal. Pressing the left end of the key steers left, and pressing the right end steers right.
7. Split: Pressing the left end of the key opens split-screen with the left screen active, or when split screen is already on, makes the left screen active. Pressing the right end of the key opens split-screen with the right screen active or makes the right screen active. Pressing the end of the key that corresponds to the active screen exits split-screen.
8. Focus: Changes the depth of the signal focus. Pressing the top of the key moves the focus up, and pressing the bottom of the key moves it down.
9. Depth: Changes the total image depth. Pressing the top of the key moves the image depth up, and pressing the bottom of the key moves it down.
10. Body Marker: Inserts body markers in the scan.
11. Text: Enables text entry and annotation on the scan.
12. PW: Enters and exits Pulsed-wave Doppler mode.
13. Color: Enters and exits Color Doppler mode.
14. 2D: Enters 2D mode.
15. CW: Enters and exits Continuous-wave Doppler mode.
16. Gain/Active: Turning the knob changes the gain. Pushing the Active button toggles between the active scanning modes and the softkeys associated with those modes.
17. Clear: Erases the currently selected annotation or measurement.
18. Calcs: Opens the Calculations menu.
19. Caliper: Starts a generic measurement. Pressing the key repeatedly cycles through available calculations.
20. Select: Chooses a trackball function. The selected function is highlighted in blue above the softkey display.
21. Cursor: Selects and displays or deselects and hides the ultrasound cursor.
22. M-Mode: Enters and exits M-Mode.
23. Zoom: Push to enter ROI box Zoom, or exit Zoom mode. Turn for Quick Zoom
24. Update: Turns updating of the 2D image on and off in PWD and CW modes.
25. Left Enter: Selects and deselects items. When the Windows screen is active, the Left Enter key acts like the left button on a mouse.
26. Trackball: Controls movement of the cursor, the ROI, and other features.
27. Right Enter: Opens context menus. When the Windows screen is active, the Right Enter key acts like the right button on a mouse.
28. Freeze: Freezes and unfreezes the scan.
29. Store: Stores a single-frame image.
30. Record: Stores a loop.

At the top left of the console is a group of system keys that control what the windows are active. They include: Patient—Opens the Patient window, Preset—Opens the Preset menu, Review—Opens the Review window, Report—Opens the Report window, End Study—Closes the current study, Probe—Opens the Imaging window; Setup—Opens the Setup window.

The keys just below the keyboard control the functions of the softkeys displayed across the bottom of the Imaging window. The softkey functions are dependent on what probe is connected, which scanning mode is chosen, and whether the scan is live or frozen. The illustrations below show examples of the softkeys when the image is live and frozen. The softkeys the system displays depend on the probe that is connected, the selected scan mode, and the selected exam. The display a user sees may differ from the illustrations shown here.

It should be appreciated that in some embodiments, the console controls may be provided via a touchscreen display rather than a being configured in a separate physical housing.

The system can include an ECG module, an ECG lead set—10 sets of electrodes, a Footswitch (Kinessis FS20A-USB-UL), a medical-grade printer and One or more transducer probes. The exemplary portable ultrasound system complies with the Standard for Real-Time Display of Thermal and Mechanical Acoustic Output Indices on Diagnostic Ultrasound Equipment (UD3-98). When the relevant output index is below 1.0, the index value is not displayed.

When operating in any mode with the Freeze function disabled, the window displays the acoustic output indices relevant to the currently-active probe and operating mode. Minimizing the real-time displayed index values allows the practice of the ALARA principle (exposure of the patient to ultrasound energy at a level that is As Low As Reasonably Achievable).

In the exemplary portable ultrasound system, to choose a scan mode, a user presses the appropriate key on the console:

For 2D, press the 2D key; for M-Mode, press the M Mode key; for Color Doppler, press the Color key; for Pulsed-Wave Doppler, press the PW key; for Continuous-Wave Doppler, press the CW key.

In the exemplary portable ultrasound system, to conduct an ultrasound exam in 2D, Color Doppler, or M-mode, the user completes these steps:
1 Load or create the patient information.
2 Press the console key for the required scan mode:
3 Press the Preset key, then select a preset from the Presets menu.

The system software loads preset image control settings that are optimized for the selected preset and the connected probe. A user can now use the probe to conduct an ultrasound exam. Refer to the appropriate clinical procedure for the exam a user are conducting.
4 If necessary, use the softkeys to adjust the image controls.
5. Press the Freeze key. The softkey controls change to allow printing, measurements, and other functions.

To conduct an exam in Pulsed-Wave Doppler mode, a user may complete these exemplary steps:
1 Conduct an exam in 2D mode,
2 Press the PW key on the console.
3 Move the range gate to the proper location, then press the Left Enter key on the console . . .
4 Use the softkeys to adjust any image control settings as needed.
5 Press the Freeze key. The softkey controls change to allow printing, measurements, and other functions.

To conduct an exam in Triplex mode, a user may complete these exemplary steps:
1 Conduct an exam in Color Doppler mode (do not freeze the scan).
2 Press the PW key on the console. The software launches Triplex mode.
3 Move the range gate to the proper location, then press the Left Enter key on the console.
4 Use the softkeys to adjust any image control settings as needed.
5 Press the Freeze key. The softkey controls change to allow printing, measurements, and other functions.

When a user switches to Triplex mode, both the original 2D scan mode and PWD mode are active. This depends on whether the options are set to simultaneous mode.

Live images are recorded by frame and temporarily stored on the computer. Depending on the mode a user selects, the system records a certain number of frames. For example, 2D mode allows a user to capture up to 10 seconds in a Cine loop.

Pulsed-Wave Doppler (including Triplex) and M-Mode scans only save a single frame for the 2D image, and a user cannot save loops for these scan modes.

When a user freezes a real-time image during a scan, all movement is suspended in the Imaging window. The frozen frame can be saved as a single image file or an image loop. For M-Mode, PWD, and Triplex modes, the software saves the Time Series data and a single 2D image.

A user can unfreeze the frame and return to the live image display at any time. If a user presses the Freeze key without saving the image or image loop, a user loses the temporarily-stored frames.

To freeze the displayed image when performing an ultrasound scan, a user presses the Freeze key. When the scan is frozen, a Freeze icon appears just above the left softkey on the imaging screen. A user can then use the Gain knob or the keyboard arrow keys to move through the frames acquired during the scan.

To start a new scan, a user presses the Freeze key again. If a user does not save the frozen image or loop, starting live scanning erases the frame data. The user saves or prints any needed images before a user acquire new scan data.

Reviewing an image loop is useful for focusing on images during short segments of a scan session. When a user freezes an image, a user can use the Gain knob to review an entire loop, frame by frame, to find a specific frame. A user can also do this when viewing a saved loop by turning the Gain knob until the desired frame displays and pressing the Store key.

To save the entire loop, a user need not select a different frame. All acquired frames are saved in the loop when a user press the Store key.

To view a loop, the user freezes the image and presses the Play softkey. The Play softkey label changes to Pause. The loop plays continuously until a user press the Freeze key or the Pause softkey. A user can track the frames and the number of the current frame in the progress bar at the bottom of the Imaging window.

In 2D and Color modes, the system can acquire loops either prospectively or retrospectively. Prospective acquisition captures a loop of live scan data following the acquire command, while retrospective acquisition saves a loop of a frozen scan.

During live imaging, pressing the Store key tells the system to acquire and save a loop of the scan following the key click. The loop displays in the Thumbnail window at the side of the Main Screen. The default length of the loop is 3 seconds, but this is adjustable, for example, between 1 and 10 seconds in the Acquisition Length section of the Setup Store/Acquire window.

When the beat radio button on the Store/Acquire tab of the Setup window is selected, and the system detects an ECG signal, the acquired loop is a number of heartbeats. A default may be 2 beats, but this also may be adjustable, such as to between 1 and 10 beats in the acquisition length section. If no ECG signal is detected, the acquired loop may be the length set in the Time field, even if the beat radio button is selected. A user can apply an R-wave delay in the Acquisition Length section. A user can also enable a beep that sounds when the acquisition is complete. The default format for loops acquired in this way is .dcm, however, they can also be saved as any of the other available formats. A user may utilize the Export tab on the Setup window to choose a different file format.

When a user views a frozen or live image, a user can use the Zoom tool to enlarge a region of the 2D image. A user cannot use the Zoom tool in the Time Series window. To zoom into the middle of the image the user:
1 Presses the Gain knob until Zoom is selected in the Gain Knob menu.
2 Turns the Gain knob to zoom in or out to the size a user want. To zoom an area that's away from the middle of the image:
   To zoom an area that's away from the middle of the image, the user:
1 Presses the Zoom Off softkey.
2 Uses the trackball to move the zoom box to the area a user want larger, and press the Left Enter key.
3 Uses the Gain knob to zoom in or out of that area.

In the exemplary portable ultrasound system, in M-mode and Spectral modes, a user can make the 2D display larger relative to the Time-Series display, and vice-versa. To resize the scanning displays:
1. Press the Setup key.
2. Click the Display tab.

To make the Time-Series display bigger and the 2D Imaging display smaller, click the S/L radio button in the M-Mode Format or Spectral Format area. To make the 2D display bigger and the Time-Series Imaging display smaller, click the L/S radio button in the M-Mode Format or Spectral Format area.
3. Click OK to apply the change.
Note: This selection applies whenever a user use the preset that was chosen when a user made the change. When a user use a different preset, the selection does not apply unless a user have also made the change in that preset.

In the exemplary portable ultrasound system, an optional image-optimization package sharpens images produced by the portable ultrasound system. The default configuration starts the software when the portable ultrasound system starts. To change this so the system starts with the optimization software off, a user may make a preset with the TV Level softkey control set to 0. The optimization software level numbers range from 0 to 3. The 0 setting applies no image processing. The larger the number, the more processing is applied to the image. To adjust the optimization level, when live imaging, a user may press the TV Level softkeys until the desired level is set.

The view options section of the general tab on the setup window lets a user add or remove several guides on the scanned image. These guides provide details about the patient. probe, and image control settings.

The system software lets a user split the Imaging screen into two sections to view two current scans for a patient. A user can acquire one scan for the patient, select Split Screen, and then acquire another scan from a different angle or location. Split Screen mode works with the 2D scanning modes (2D and Color Doppler).

When a user enters split screen mode, the system software copies the current settings for the Image Control window to the new screen. A user can then apply any Image Control setting independently to either screen. A user can go live or freeze either screen (only one screen can be live at a time), and a user can use any of the tools and menus with either screen. In addition, a user can scan in different modes in each screen. For example, a user can acquire a 2D scan, enter split screen mode, then acquire a Color Doppler scan in the second screen. The following figure shows an example of a split screen.

The active screen has cyan bars at the top and bottom. To activate the other screen, a user performs one of these actions:
Move the arrow cursor to the desired screen and press the Left Enter key.
Press the Toggle Screen softkey. To exit split screen mode, use any of these methods:
Press the 2D key.
Select a different exam
Select M-Mode, PWD, or Triplex scan modes
Press the Split softkey
   When a user exits Split Screen mode by pressing the Split softkey, the system software keeps the acquired data for the active screen (the one with the cyan lines at the top and bottom) and discards the acquired data for the other screen.

Text mode lets a user add text and symbols to an image, using the softkeys. Softkey controls that are available in Text mode include:

Laterality places the word Left or Right on the image. Pressing the Laterality softkey cycles between Left, Right, and no text.

Location opens a menu of body locations, or increments through a list of body locations. If a menu opens, the appropriate item may be clicked to place it on the image.

Anatomy opens a menu of names for different anatomies, or increments through a list of anatomies. If a menu opens, click the appropriate item to place it on the image.

Orientation opens a menu of patient orientations, or increments through a list of patient orientations. If a menu opens, click the appropriate item to place it on the image.

Body Marker opens the Body Marker menu.

Text New starts a new line of text at the home location.

Text Clear deletes all text (including manually typed text and arrows) from the image

Home moves the text cursor or selected text to the text home position.

Arrow places an arrow at the text home position, or if there is text on the image, at the middle of the last line of text

Set Home sets the text home position. Move the text cursor to the desired location, then press the Set Home softkey.

To enter text mode, press the Text key. The system software places a text cursor (I-beam) on the Imaging screen. The trackball is used to move it to where a user want the new text, and either type the text, or use one of the Text-mode softkeys. When the text is done, press the Left Enter key. If a user added custom text using the Annotation tab of the Setup window, that text shows in the softkey list to which it was added.

A user can also add predefined text, using the softkeys. This lets a user add labels and messages a user needs often, without having to type them each time.
1. Press the Text key on the console, or press the Space bar on the keyboard.
2. Press one of the softkeys for predefined text:

Laterality places the word Left or Right on the image. Pressing the Laterality softkey cycles between Left, Right, and no text.

Location opens a menu of body locations, or increments through a list of body locations. If a menu opens, click the appropriate item to place it on the image.

Anatomy opens a menu of names for different anatomies, or increments through a list of anatomies. If a menu opens, click the appropriate item to place it on the image.

Orientation opens a menu of patient orientations, or increments through a list of patient orientations. If a menu opens, click the appropriate item to place it on the image. Selecting an item with one of the softkeys places it on the image.

A user can place two kinds of arrow on a frozen image: marker arrows and text arrows. The default is marker arrows. A user can place as many arrows as a user want on an image. Marker arrows are short, hollow arrows that indicate a spot on the image. When a user places an arrow (see the procedure below), the arrow is green. A user can use the trackball to move the arrow while it is green. A user can select an arrow by clicking on it. When an arrow is selected, a user can move it with the trackball and rotate it by pressing the Select key, then moving the trackball. To place a marker arrow on an image, complete these steps:
1 Press the Arrow softkey.
2 Use the trackball to move the arrow to where a user want it
3 To rotate the arrow, press the Select key and move the trackball.
4 To place another arrow on the image, press the Arrow softkey.
5 Press the Left Enter key to set the arrows and exit Text mode.
   Text arrows are dashed-line arrows that a user can draw from text to a point on the scanned anatomy. A user can also add an arrow without adding text. To use text arrows, a user must make a selection on the Setup/Annotation window.

After placing text on an image, a user can easily move it to any location within the Image Display. To move text, click the text, move it to a new location, and press the Left Enter key. If an arrow is attached to the text, the origin of the arrow also moves.

A user can add an icon to the 2D image that identifies the anatomy of the scan. Body Marker in the Annotation menu opens a window containing several anatomical views based on the current exam. To add a body marker to an image, a user completes these steps:
1 Press the Text key.
2 Press the Body Marker softkey. A body marker displays on the image.
3. If the marker a user wants is not displayed, press the Next Marker or Prev Marker softkey. If another marker is available, it replaces the first marker.
4. When the marker a user want displays, press the Left Enter key.
   To change the body marker, complete these steps:
1. Click the body marker. The marker turns green and the softkeys change to the Body Marker set.
2. Press the Next Marker or Prev Marker softkey.
3. When the marker a user want displays, press the Left Enter key.
   A user can move the body marker to any location on the image. To move the body marker, complete these steps:
1 Click the body marker to select it.
2 Press the Marker Position softkey.
3 Use the trackball to move the body marker.
4 When the marker is where a user want it, press the Left Enter key twice.
   A user can move the orange probe indicator to anywhere on the icon to more precisely indicate the scanned anatomy.
   To move the orange marker, complete these steps:
1 Click the body marker. The text above the softkey display changes to show Probe Pos is selected.
2 Use the trackball to move the probe indicator to the desired location on the body marker.
3 When the marker is where a user want it, press the Left Enter key.
   To rotate the probe indicator to more positions complete these steps:
1. Move the Windows pointer over the body marker. The pointer changes to pointing hand.
2 Press the Select key to highlight Probe Orient in the line above the softkey display.
3 Use the trackball to rotate the probe indicator to the desired orientation on the body marker.
4 Press the Left Enter key to lock the indicator in position.

A set of softkey controls below the Imaging window display the currently available imaging controls. The softkeys are operated by the keys on the console or alternatively using a touchscreen display. When a user select a scan mode, the software configures the softkeys for that mode. The controls displayed vary depending on which probe is connected, and on other selections. Pressing the left and right arrow keys at the left side of the console changes the display to other controls available in the selected mode.

To change a setting, use the toggle keys on the console. Each toggle key controls the setting in one of the softkeys at the bottom of the Imaging window. The position of the key set corresponds to the position of the onscreen button—the leftmost key controls the setting in the leftmost softkey, and so on.

FIG. 43 illustrates softkeys 6420 shown as an example of available 2D image controls. A user can only adjust these image controls during live scanning. When a user freezes a scan, the system software replaces the softkeys with a different set, for printing and making annotations and measurements on the scan image.

The softkey display depends on the probe that is connected, the selected scan mode, and the selected exam. A user can adjust the following 2D image controls during live scanning: Frequency, Scan Depth, Focus depth, Gain, Time Gain Compensation (TGC), Image Format, Omni Beam, Left/Right and Up/Down invert, Colorization, Persistence, Image map, Needle guide, Dynamic range, Software optimization controls.

When a user selects an exam, the system software sets an appropriate frequency for that exam. A user can select an alternate frequency to better suit specific circumstances. In general, a higher transmit frequency yields better 2D resolution, while a lower frequency gives the best penetration. To select high, medium, or low frequency, use the Frequency softkey. The exact frequencies vary, depending on the connected probe. Each frequency has a number of other parameters associated with it, which depend on the type of exam. The selected frequency shows as H, M, or L in a character string in the information to the right of the Imaging window. In the example below, medium frequency is selected.

The Depth key adjusts the field of view. A user can increase the depth to see larger or deeper structures. A user can decrease the depth to enlarge the display of structures near the skin line, or to not display unnecessary areas at the bottom of the window. When a user selects an exam type, the system software enters a preset depth value for the specific exam type and probe. To set the scan depth, use the Depth key. After adjusting the depth, a user may want to adjust the gain, time gain compensation (TGC) curve, and focus control settings. A user can view a depth ruler on the image by selecting Depth Ruler on the General tab of the Setup window.

In accordance with various embodiments, the handheld housing associated with portable or tablet ultrasound devices described herein can have compact form factors. For example, the handheld housing of the tablet ultrasound device can provide a diagonal dimension for the touch screen display in a range of 8 inches (˜20 cm) to 18 inches (˜46 cm). In some embodiments, the electronic components to operate the ultrasound and computer are designed using a 3D board architecture to enable more compact placement of components within a housing of smaller size.

FIG. 44 illustrates a cross-sectional view of a tablet ultrasound device 2000′ according to various embodiments wherein the tablet's motherboard 106′ and ultrasound engine 108′ are stacked vertically over one another rather than being placed side-by-side. In other words, the motherboard 106′ and the ultrasound engine 108′ are constructed and arranged according to three-dimensional system architecture principles. In some embodiments, the motherboard 106′ and the ultrasound engine 108′ are connected using a board connector 7001. The board connector 7001 can provide at least partial mechanical support for the motherboard 106′ and/or ultrasound engine 108′ in some embodiments. In some embodiments, electrical connections between components of the motherboard 106′ and components of the ultrasound engine 108′ can pass through the board connector 7001.

FIG. 45 illustrates a bottom schematic view of the tablet ultrasound device 2000′ with the bottom portion of the housing and the ultrasound engine 108′ removed. The view thus shows the inverted motherboard 106′. The motherboard 106′ includes a processing unit 7002, a memory 7004, the board connector 7001, data storage 7006, a cooling fan 7008, a battery 7010, and a trusted platform module 7012. In preferred embodiments, memory 7004 can comprise a shared memory device mounted on a second circuit board mounted above, or below, a first circuit board, or can comprise a layer in a stacked plurality of circuit layers to provide a three dimensional (3D) circuit device. In some embodiments, the data storage 7006 can include solid-state drive storage (i.e., drive storage with no moving parts). The processing unit 7002 can contact heat dissipation pipes in some embodiments to remove excess heat from the vicinity of the processing unit 7002. The motherboard 106′ can interface with external devices such as transducer probes, data storage devices, or external displays as described above using connectors 7014. In some embodiments, the motherboard 106′ can include one or more connectors 7014 to interface with one or more external devices using communications standards such as universal serial bus (USB 1.0/2.0/3.0, USB-C, miniUSB, microUSB), DisplayPort and Mini DisplayPort, Lightning, Thunderbolt, high-definition multimedia interface (HDMI), or other appropriate standards or protocols.

The trusted platform module (TPM) 7012 comprising an encryption and decryption circuit can interface with other motherboard 106′ components (such as the data storage 7006, the memory 7004, and display drivers) to secure and encrypt data on the tablet ultrasound device 2000′. The TPM 7012 can encrypt all data written to the data storage 7006 and the memory 7004 in real time and can decrypt all data retrieved from the data storage 7006 and the memory 7004 in real time. In some embodiments, the TPM 7012 can encrypt one or more data fields in each packet of data. By providing real time encryption and decryption, the TPM 7012 ensures that sensitive patient data is always encrypted in any storage medium on the device. As a result, patient data cannot simply be extracted from the memory 7004 or the data storage 7006 in the event that the tablet ultrasound device 2000′ is lost, stolen, or decommissioned.

FIG. 46 illustrates a schematic view of the display of the tablet ultrasound device 2000′ in accordance with various embodiments described herein. The tablet ultrasound device 2000′ can utilize a mode switching menu 7030 that can be operated by touch control in some embodiments. When the mode switching menu 7030 is activated by touch, the display provides the user with a variety of operation modes 7032. The mode switching menu 7030 can enable a user to select from among the variety of operation modes 7032 to enable fast switching of the device among different imaging or image analysis modes. In some embodiments, the operation modes 7032 can each be based upon different machine learning algorithms or other computer aided diagnostic functions.

In some embodiments, the tablet ultrasound device 2000′ can be responsive to voice commands. A voice indicator 7020 can appear on the display when the device 2000′ is actively listening for voice command or control. Voice indicator 7020 can also be touch activated to turn on or off the voice actuated operation. In such embodiments, the tablet ultrasound device 2000′ can include a microphone to detect a user's voice that is embedded within the tablet housing. In other embodiments, the tablet ultrasound device 2000′ can receive wired or wireless signals corresponding to voice commands received from an external source, e.g., headphones or a microphone worn or used by the user. In some embodiments, voice commands can provide the most practical method of control and adjustment for features of the device 2000′. For example, a user within a magnetic resonance imaging suite may be able to use the transducer probe on a patient near the magnetic bore but may not be able to place the tablet device housing near the magnetic bore. In such a case, the user may use voice commands to remotely control functions on the tablet ultrasound device 2000′ from a distance while the tablet ultrasound device 2000′ is located in a safe place away from the magnet.

Many functions on the tablet ultrasound device 2000′ can be operated using voice. Upon voice activation, the voice indicator 7020 may animate or, for example, change color or shape to indicate that a voice command has been received or acted upon. In various embodiments, the user may provide the device with voice commands, e.g., “Gain up,” “Contrast down,” etc., that the device can then implement. In some embodiments, the device 2000′ can include present values or changes that will be implemented upon actuation by voice command. For example, a command to “gain up” may increase gain on the image by a preset amount such as 10%.

The above devices and methods can be used with conventional ultrasound systems. Preferred embodiments are used in a touchscreen actuated tablet display system as described herein. Touch actuated icons can be employed such that gestures can be used to control the imaging procedure.

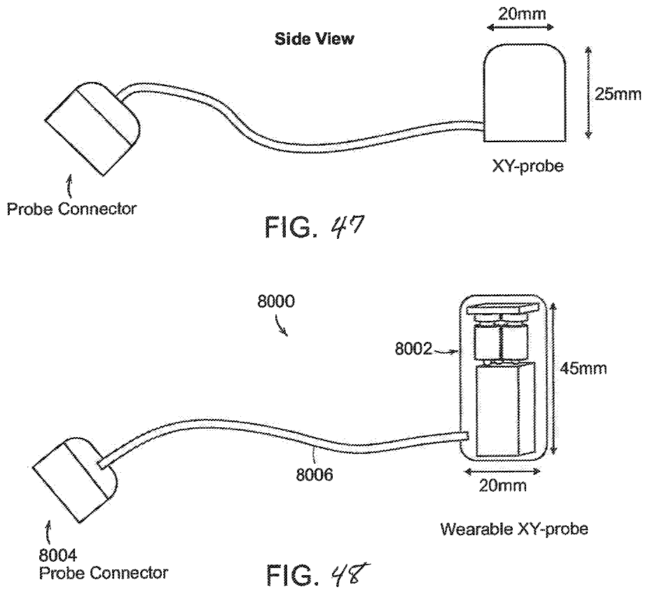

A wearable XY-probe, (or alternatively, a 2D transducer array) as described herein can be, as in this example, an 18 mm×18 mm XY-acoustic module can be positioned within a housing 8002, such as a plastic flat top and bottom package, with a cable 8006 coming from the side, see for example, FIGS. 47 and 48. The cable 8006 (or wireless connection) extending to probe connector 8004, enables delivery of control signals to the transducer probe 8002 from a tablet or other ultrasound control system, or transmission of ultrasound data or signals from the probe assembly to the ultrasound tablet or system.

The probe can be taped, coupled or attached to the patient's chest, to thereby continuously monitor the heart function, cardiac output, etc. A harness or similar transducer coupling device or element can be used to position the transducer such that the transmissive coupling media, such as a gel pad, is suitably coupled to the required position relative to the patient's heart. In order to be able to tilt the transducer module, a MEMS or VCM magnetic actuator can be mounted on top of the acoustic module to provide the tilt. In attaching the wearable probe to a patient's body, a standoff pad can be used to provide acoustic coupling between the ultrasound transducer (probe) and the patient's skin. Standoff pads are made of a soft compliant material such as a gel pad. The gel pad has the ability to conform to a hard, noncompliant surface such as ribs on the chest. Without a standoff pad, the rigid surface of a probe may not conform to the patient's chest, which can create gaps or spaces between the transducer and the chest. Such gaps yield artifacts and poor image quality during diagnostic ultrasound scanning. The acoustic and actuator assembly, as shown in FIG. 77, is packaged in a plastic housing. The acoustic and actuator assembly is proposed to be taped onto the skin of the patient to transmit and receive through a standoff pad. The wearable probe can be used to do continuous apical view ejection fraction measurement, and also can be used to measure the cardiac output using either apical view or parasternal long axis view. An advantage of the disclosed XY-probe is that the probe provides parasternal long axis and short axis view simultaneously, so a user can measure the LV stroke volume based on pulsed-wave doppler long axis view and at the same time use the short axis view to measure the mitral view area.

The wearable XY-probe taped on a patient to monitor the heart function can comprise a wearable acoustic module having a square 18 mm×18 mm front face, for example, and side exit of small coax cable assembly. A micro-electromechanical system (MEMS) or magnetic actuators can be integrated on top of the acoustic module to provide tilt function.

A thermocouple can be placed inside the probe to monitor the probe temperature. A pressure sensor can be mounted on the probe to monitor how much force is needed to couple it on the patient such as by tape, harness, etc.

Systems and methods described herein enable simultaneous display of 4 channel and 2 channel apical views to measure the LV volume for ejection fraction (EF) measurement and for diastolic filling time (DFT) measurements. Additionally, an apical view or simultaneous parasternal long axis view and short axis view for cardiac output measurement can all be utilized by touch actuation on the touchscreen or other user interface.

Diastolic heart failure, a major cause of morbidity and mortality, is defined as symptoms of heart failure in a patient with preserved left ventricular function. It is characterized by a stiff left ventricle with decreased compliance and impaired relaxation, which leads to increased end diastolic pressure. DFT, Diastolic Filling Time, a critical LV function diagnostic indicator is the period of the cardiac cycle that encompasses ventricular relaxation, passive and active filling of blood into the heart, and the period just prior to ejection. It is the period in which the ventricle fills with blood from the left atrium. The XY-probe provides simultaneous apical 4-CH and 2-CH view, it allows continuous measurement of Left Ventricle Volume, allows LV volume displayed as a function of time. Once the LV is displayed as a function of time, it is straightforward to measure the diastolic filling time as the time between minimum LV volume to maximum LV volume during the heart cycle.

For the wearable XY-probe, there are at least three critical continuous echocardiography measurements available: 1. Cardiac Output, 2. Auto EF, and 3. Diastolic Filling Time (DFT). These echocardiographic measurements generate data that can be delivered to the ultrasound system for display and diagnostic purposes.

The following describes a transducer tilting mechanism using a magnetic actuator (the same design concept can be implemented using MEMS or other types of actuators) followed by cardiac output measurement and Auto-EF and DFT measurements.

Optionally, an electronically controlled tilting mechanism can be added to the wearable XY probe to adjust the transducer tilting angle while attached to the patient. This system can be used to manually or automatically control the orientation to enable periodic adjustment or remote control of the beam direction of the transducer. Thus, a sonographer or care giver does not have to be present during monitoring operation even when the patient moves such as by breathing or changing position. Such “hands free” operation of the transducer assembly can significantly improve and simplify ultrasound treatment or diagnostic operations of ultrasound systems.

The mechanism comprises three linear motors in some embodiments. Each motor can be electrically controlled to extend or contract linearly along its center axis. An example is a voice coil actuator (e.g., H2 W Technology Inc., part #NCC01-04-001-1X). The choice of motor is for illustration purposes and other options can also be used. Embodiments of this disclosure may be implemented with other commercial standard or custom linear motors or MEMS actuators, for example. The transducer motion actuator can perform probe rotation about the beam transmission axis, rock the beam transmission axis to shift along a selected direction or tilt to change the size.

FIGS. 49A and 49B demonstrate that the axial position of a linear motor can be extended under the control of an actuator to rotate the plane of the beam transmission axis in any desired orientation. The spacing between the two, three or more contact points for the actuators can be selected to define the range of motion of the central transmission axis of transducer. A first actuator 8010 can be set at a first displacement amount and a second actuator 8012 can have a second (larger) displacement amount (seen at 8005). Each actuator can have a cap 8007 that defines or is connected to a back plane for the transducer assembly. A central post 8005 extends into a displaceable actuator element 8009 having to contact element or point 8011, in which a combination of such points operates to set the tilt angle of the transducer two or three dimensions.

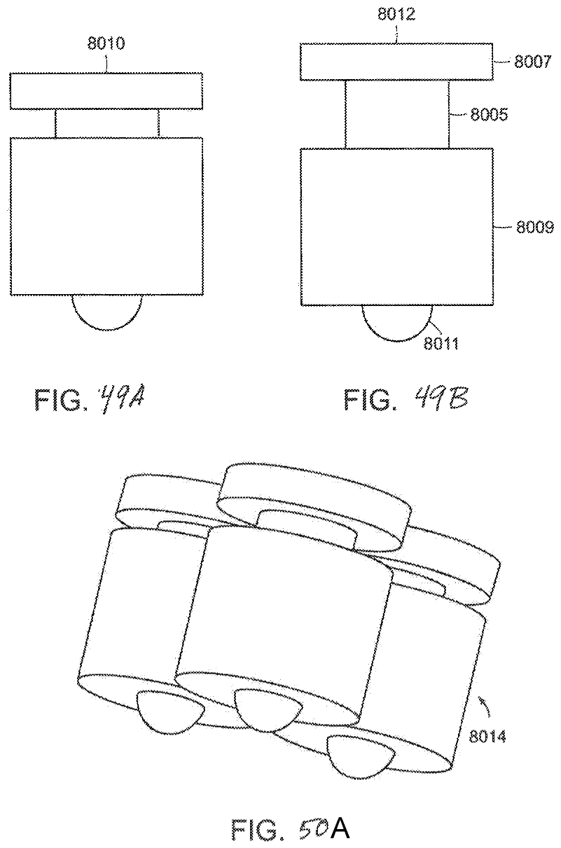

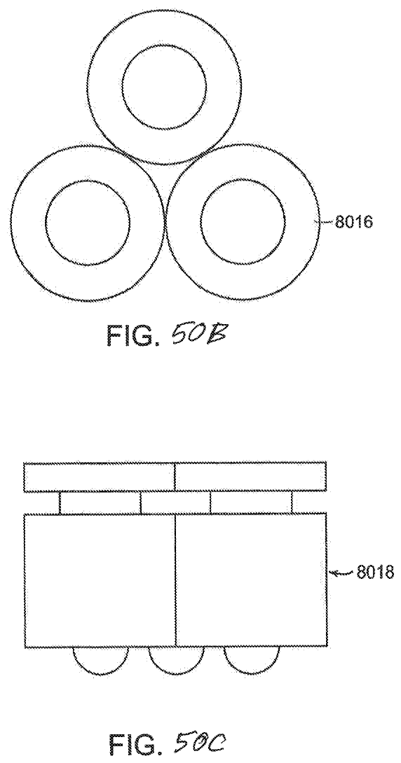

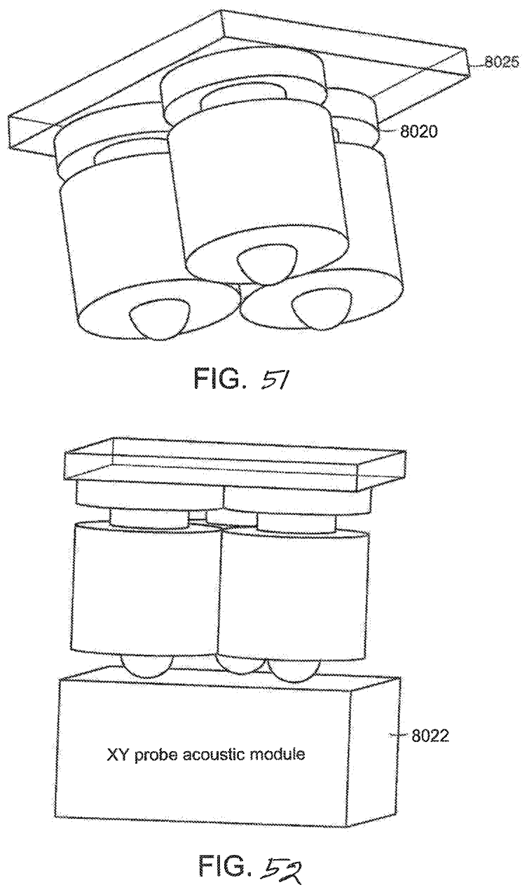

As an example to demonstrate the tilting of a XY probe by the actuators, three actuators 814 can be used and packaged together as shown in FIGS. 50A-50C. The tops of the three motors are mounted to a flat plate or backplane 8022 as the reference for tilting as shown in FIG. 51. The bottoms 8016 of the three motors 8018 aligned along a common axis are mounted on the top surface of the XY probe transducer module 8025 and press down on the XY acoustic module by the tension of the transducer coupling element (tape, harness etc) that attached the entire assembly to the patient body, see FIG. 52.

When all three motors are at the rest position, i.e., extended by zero mm, the XY transducer acoustic module lies flat relative to the top position reference plate. The XY transducer acoustic module is tilted when the three motors have different degrees 8024 of linear extension as shown in FIG. 53. The tilting has three degrees of freedom, limited by the maximum length extension of the individual motor. By programming the three motors to have different extended lengths 8026 (see FIG. 54) along the z-axis that is orthogonal to the backplane 8022, the XY probe acoustic module can be tilted due to the different length of the three actuators. This can define a cone through which the central transmission axis can be displaced.

An example of using the XY-probe at an apical for cardiac output measurement is described next. First, measure the LVOT diameter. Zoom in to be accurate. Measure up to 0.5 cm back from the aortic valve leaflet insertion points (on the ventricular side).

Second, using pulse wave Doppler (PW), line up the LVOT in the apical views, using either the apical 5 chamber or the apical 3 chamber. Aim to be as close as possible to the aortic valve, but not into the area of flow acceleration. The flow of blood is laminar through the PW gate, which is why the all the velocities follow a narrow band and the PW waveform is not “filled in”. The PW gate can be 2-4 mm, for example.

Third, obtain the PW waveform. To get the most accurate reading, move sample volume toward aortic valve until flow accelerates. Then move sample volumes slightly away from the aortic valve, toward apex until laminar flow returns.

In a surface echo, the blood flows through the LVOT away from the probe so the curve is below the line. It should look hollow if the blood has laminar flow. Trace along the edge of the modal velocity (the outside of the chin, not the beard of the waveform) to measure the area under the curve (the Velocity Time Integral—VTI expressed in cm).
1. The Left Ventricular Outflow Tract (LVOT) is assumed to be roughly circular. Measure a diameter and you can calculate the area of the circle.
2. Pulsed Wave Doppler (PW) through the same point, in the center of the LVOT tells us how fast that blood is travelling at any time.
3. The area under the curve then tells us how far the column of blood has been pushed, (V axis is in m/sec, X axis in seconds, so the area ruder the curve will tell us how far the blood has moved travelling at these velocities for this amount of time.)
4. Work out the volume of the cylinder—Multiply the area of the LVOT (a circle) by the length the blood travels and you get the stroke volume (ie volume ejected per beat)
5. The stroke volume multiplied by the heart rate gives us the cardiac output (expressed as L/Min).
6. Divide the cardiac output by the body surface area and we get the Cardiac Index.

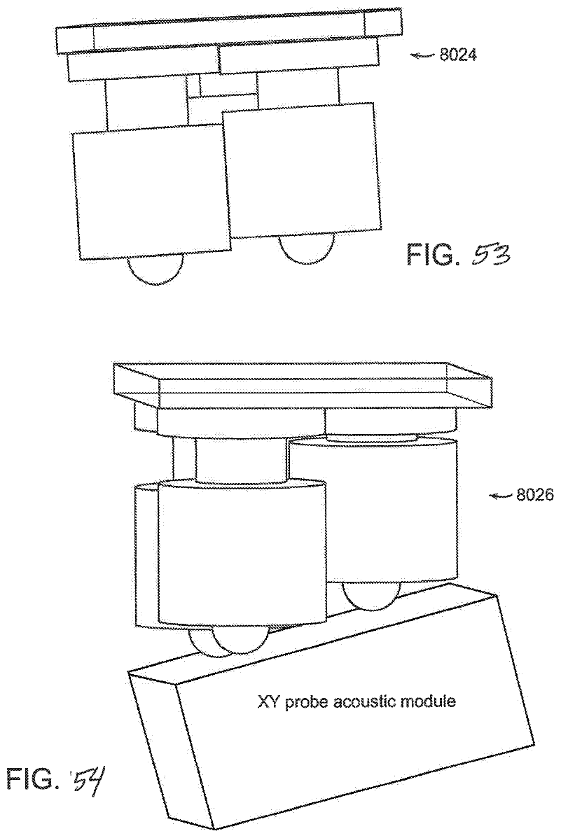

Illustrations of cardiac LVOT output measurements in apical view 8040 and in parasternal view 8046 are shown in FIGS. 55 and 56, respectively, wherein the transducer probe 8002 can be used to set a gate 8042 at one or more of the left ventricle (e.g. mitral valve or MV), the right ventricle (tricuspid valve), or alternatively, a gate 8044 at the valve for the left ventricle, for example. A touch actuated tilt control 8090 can be included in the touchscreen control features as shown in FIG. 57.

XY-Probe Demonstration: EF Measurement in Apical View

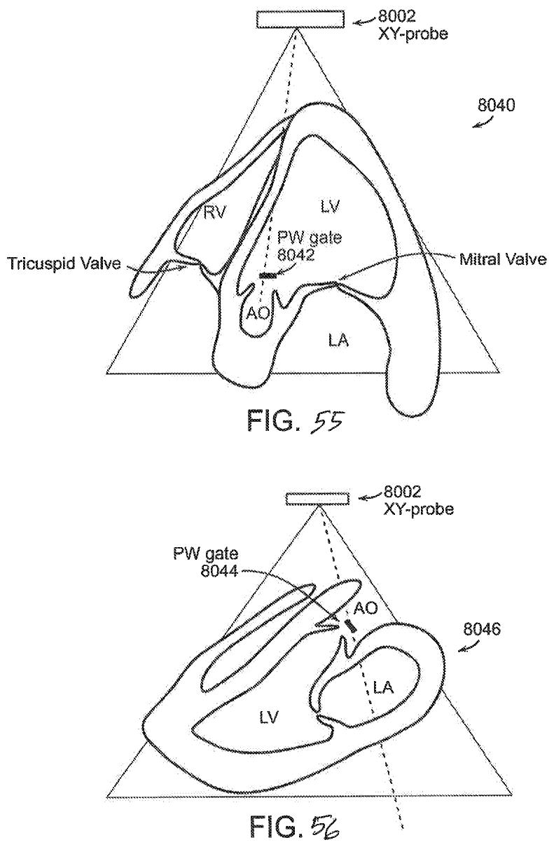

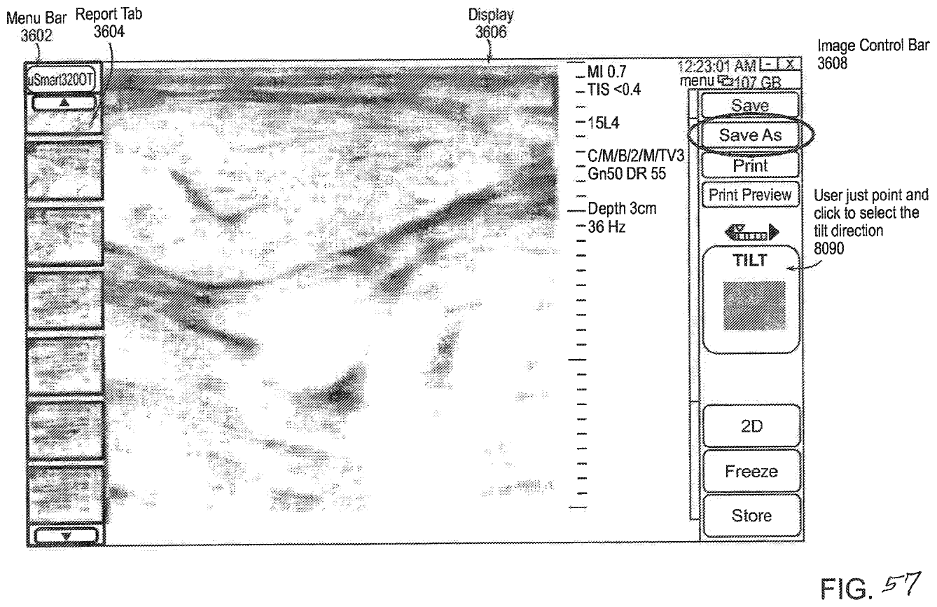

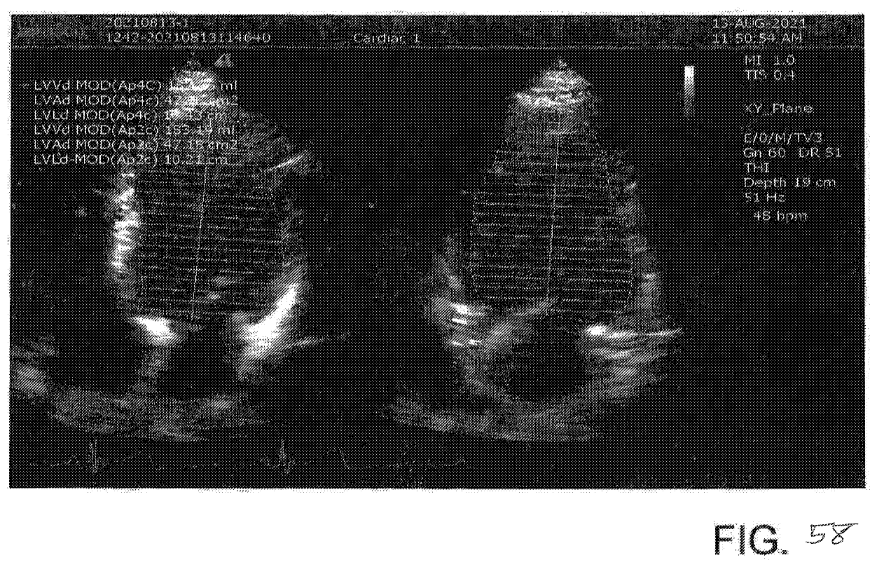

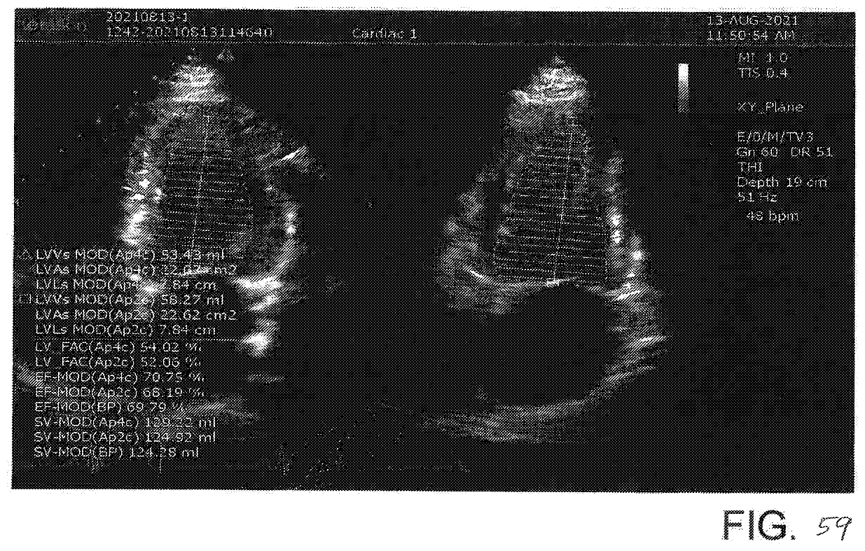

The XY probe can be used to do EF measurement based on the apical view Simpson method. The biplane-probe provides visualization of two orthogonal planes and ensures on-axis views are obtained. As depicted in FIG. 58, an XY probe acquires real-time simultaneous images from two orthogonal planes and the images are displayed on a split screen. A manual contour tracing or automatic boarder tracing technique can be used to trace the endocardial boarder at both end-systole and end-diastolic time from which the EF is calculated. Note that the area for each of the stacked slices seen in FIG. 58 is used to compute the volume of each slice can be measured and these are summed to obtain the total volume. The LV areas in the apical 2CH, and 4CH, views, A1 and A2 respectively, are measured at the end of diastole and the end of systole. The LVEDV, left ventricular end-diastolic volume, and LVESV, left ventricular end-systole volume, may be calculated using the formula:

$$V = \frac{8}{3 π} \frac{{A}_{1} {A}_{2}}{L}$$

where L is the length of the LV, and the ejection fraction may be calculated by

$$EF = \frac{EDV - ESV}{EDV} \times 100$$

A measured 4-chamber and 2-chamber Simpson result is shown in FIG. 58 and FIG. 58, respectively. As can be seen on FIG. 59, the biplane (BP), EF calculation based on the apical view 4C and 2C LV volume measured at the end Diastole and systole, respectively, is 69.79%.

XY-Probe Demonstration: EF Measurement in Parasternal View

Left ventricular ejection fraction (LVEF) is important for characterization and management of patients and selection of therapy. The Teichholz formula, Vol=7D³/(2.4+D), is widely used, as it calculates LV volume using only LV diameter D2, see FIG. 60. For Teichholz, D may be measured from the LV short-axis slice just basal to the papillary muscle tips at end-diastole and end-systole. Left ventricle volume may be calculated using only LV diameter (D) based on the equation:

$$Vol = \frac{7 {D}^{3}}{2.4 + D}$$

The LVEF EF % may be calculated by:

$$EF = \frac{EDV - ESV}{EDV} \times 100$$

where EDV is the end diastole LV volume and ESV is the end systole LV Volume.

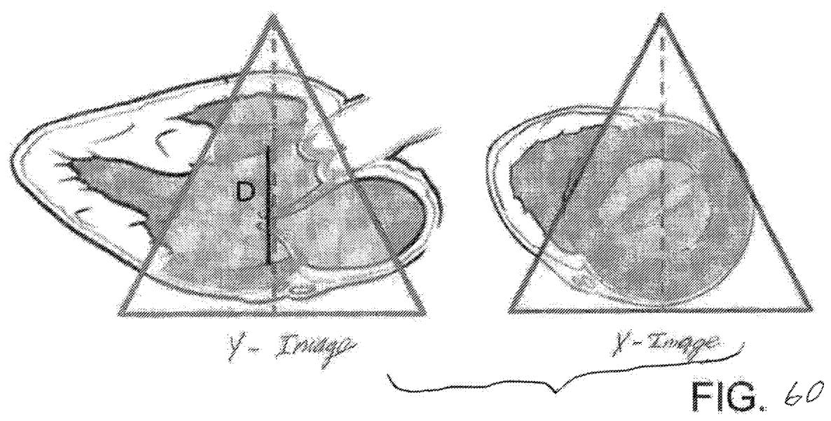

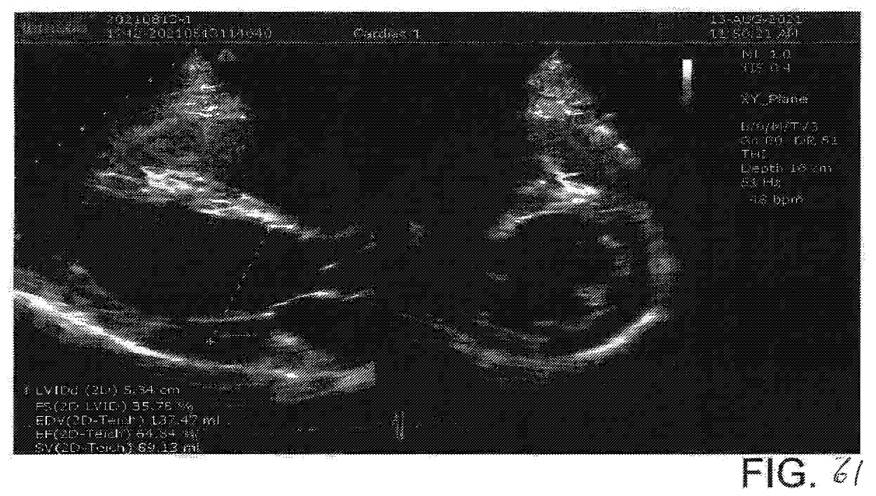

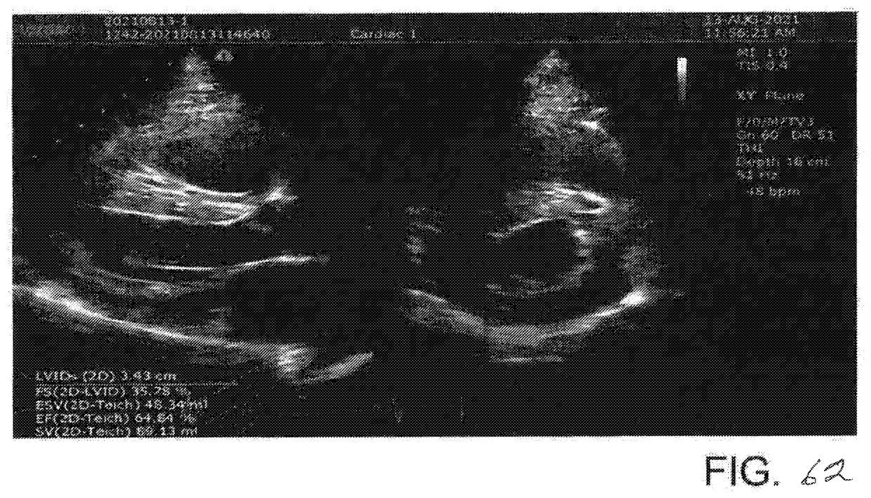

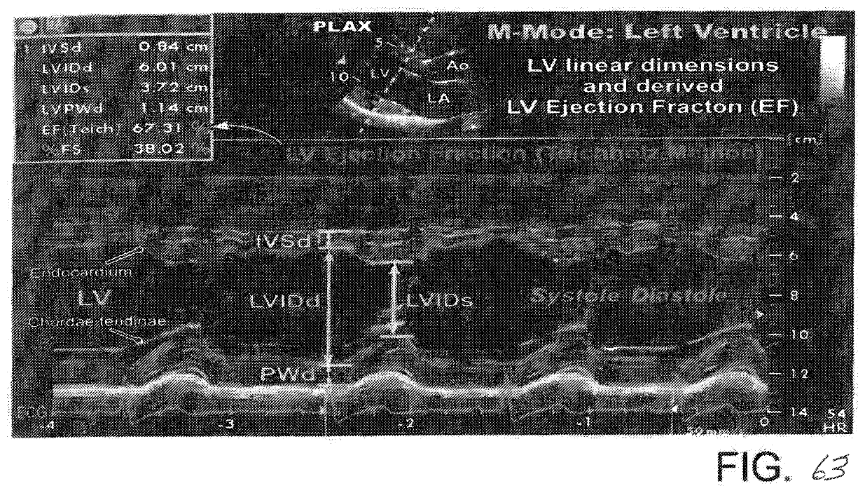

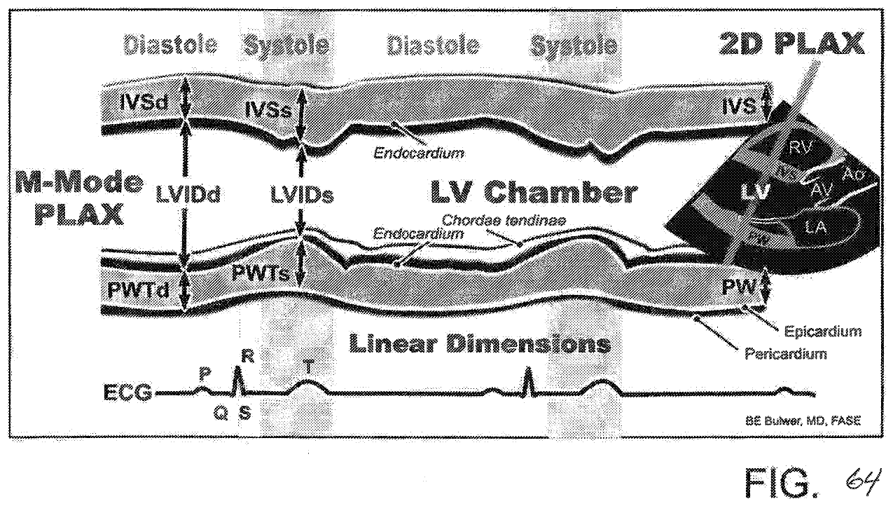

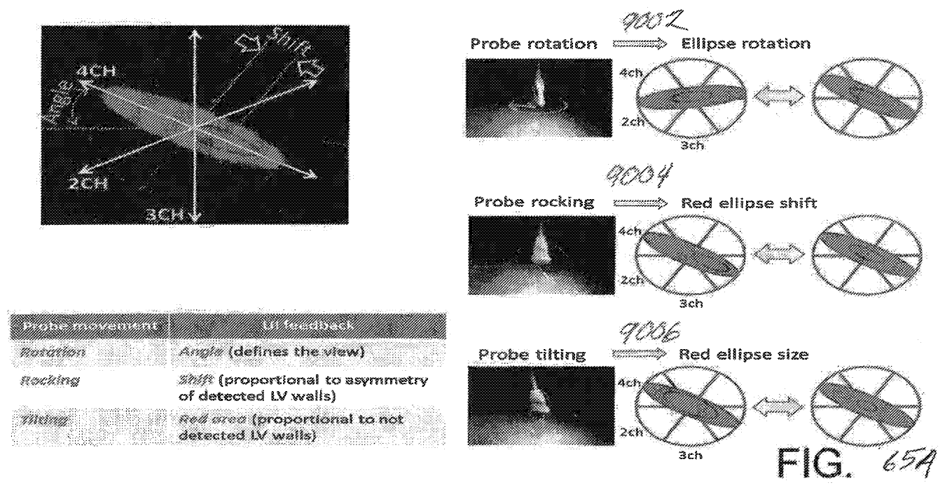

The XY-probe may be used to measure the LVID_{d} and LVIDₛ as shown below in FIGS. 61 and 62. The calculated LV volumes based on the measured LV diameter at the end of diastole and systole are used to calculate the LV EF. For example, as shown in FIG. 62, the measured LV EF based on Teichholz equation is 64.84% The LV EF measurement based on Teichholz measurement in the parasternal view and the LV measurement based on the biplane Simpson method in the apical view are very close, 64.84% vs 69.79%, respectively.

An M-Mode Auto EF Measurement

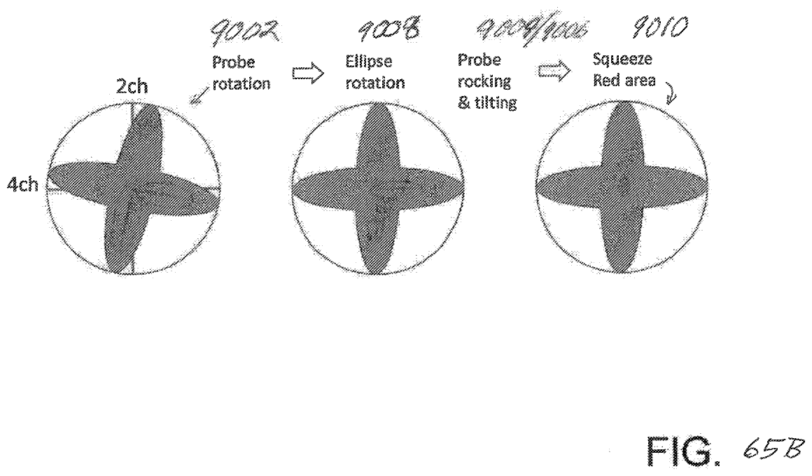

The XY-biplane probe described herein offers simultaneous real-time acquisition and display of two orthogonal echo planes from a single acoustic window. In one embodiment, the wearable XY-probe may be used to provide continuous auto-EF measurement by using anatomic M-mode to automatically and continuously measure the LVIDd and LVIDs, so as to calculate the diastole and systole LV volume and then the EF as shown in FIGS. 63 and 64.

An exemplary cardiac Teichholz LVEF measurement made in parasternal view, using the XY-probe may be manually controlled:
1. After a parasternal view scanning is completed, the operator reviews the saved video.
2. The end-diastolic frame is selected and LV internal diameter measured during diastole (LViDd)
3. The end-systolic frame is selected and LV internal diameter is measured during systole (LViDs).
4. Software uses the measured LViDd and LViDs to calculate the left ventricle volume at end diastole and the end systole, respectively.
5. Software uses the measured LV volumes to calculate the ejection fraction.

The 2D PLAX view allows proper line placement for anatomic M-mode and allows the continuous monitoring of the LV inner diameters in parasternal view (see insert in FIG. 64). As a result, the same XY-probe described previously when packaged in a wearable format allows the device to be used as a cardiac auto-EF monitoring device. One of the critical factors for the success of this Teichholz auto-EF monitoring implementation is based on the possibility that a novice user can place the wearable XY-probe on a patient's chest to acquire a 2D PLAX view with proper probe orientation, proper rotation, and probe tilt. Then, anatomic M-mode can be used to continuously monitor the LV inner diameter.

Scan guidance is important for the XY probe due to unique image presentation and lack of experience among non-cardiologists and novice users such as EMSs, Anaesthesiologists, EDs, technicians, etc.

Simultaneous analysis of two scan planes, the parasternal long axis, PLAX, and parasternal short axis, PSAX, provides unique visual clues of the valvular motion, LV wall synchrony and the critical cardiac structure and functions. Embodiments provide a simple graphical interface to allow intuitive operation of the probe to obtain correct PLAX and PSAX images with good IQ.

For good imaging in both planes, the orientation of the XY probe is different from that of the single plane probe.

In one embodiment, an exemplary scan guide tool performs the following:

Optimized acquisition that facilitates both visual assessment and automatic quantification;

View recognition (4CH, 2CH); PLAX (parasternal Long axis), PSAX (parasternal short axis).

The user is able to adjust the probe position and orientation in multi-parameter space and perform:
1. Rotation
2. Rocking
3. Tilting
4. Position along the ribs
5. Position in an inter-rib space

Scan Guide in Apical Views Algorithm

In one embodiment, the scan guide tool includes an apical view algorithm that performs image analysis to detect physiological landmarks including LV Walls, MV leaflets and LVOT. A landmarks analysis allows view classification and detailed quality estimation. For LV Walls, the percentage of detected left (RV free) wall and middle wall pattern discriminate PLAX views. The lengths and positions define IQ and LV visibility while wall inclinations determine LV rotation. For LVOT+walls, the detected LVOT indicates good PLAX. For MV leaflets, the MV position defines scan depth, the MV leaflets determine good PLAX and an LA analysis of the image below MV is performed.

In an embodiment, the scan guide tool provides a convenient and intuitive user interface (UI), that allows the user to adjust the probe on-the-fly in response to UI feedback and provides a continuous graphical presentation.

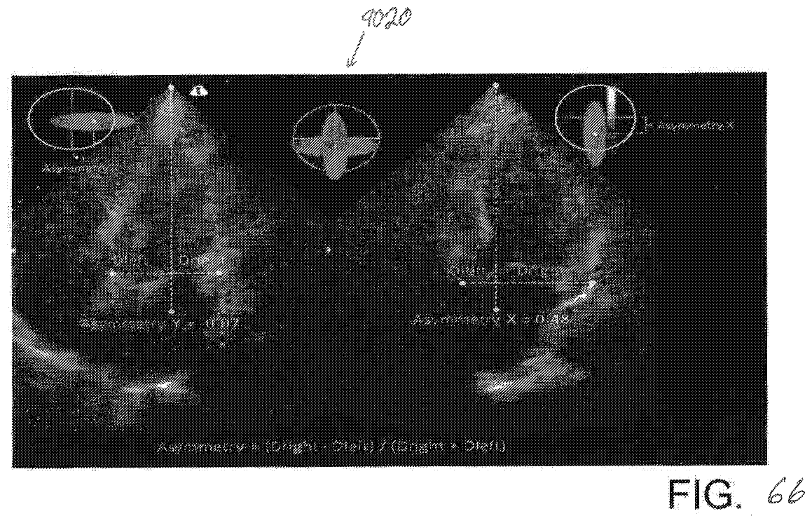

In one embodiment, probe movements and UI feedbacks are split into a minimal set of almost independent groups such as rotation, rocking and tilting as depicted in FIG. 65A. Probe rotation 9002 leads to ellipse rotation 9008, probe rocking 9004 to a color-coded (e.g. red) ellipse shifting 9008 and probe tilting 9006 to a color-coded ellipse changing size. The UI can be configured so that the user may manipulate the probe so as place a color-coded ellipse in the center and minimize 9010 the colored area as feedback about correct probe position (see FIG. 65B). In an embodiment, the scan guide may provide simultaneous analysis of both scan planes and view detection in both planes (4CH, 2CH, 3CH, 5CH) to define the probe rotation. The quality of wall detection defines the red/colored ellipses shift and size while intuitive probe manipulation allows acquisition of preferred 4ch and 2ch views and improves the IQ such that a selected metric correlated with proper transducer position based on a selected threshold (e.g. size of red/colored area) can be communicated to the user by visual (on the display and/or light signal on the handheld transducer housing) or sound indicator.

UI Guidance

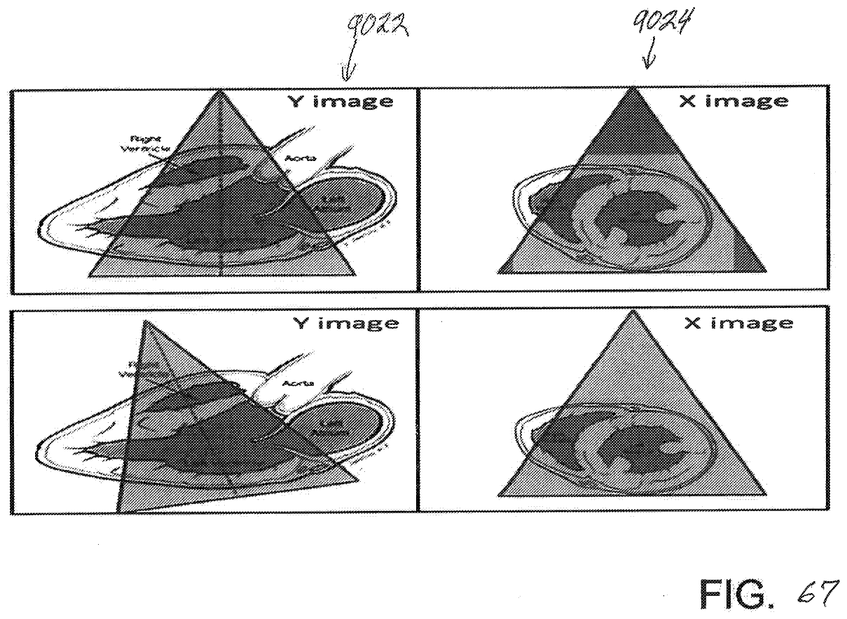

In an embodiment depicted in FIG. 66, crossed ellipses correspond to crossed scan planes, and areas indicated with color may mark the asymmetry of the probe position such that slight movement of the probe brings the ellipses into the center of the circle while probe rocking brings a red or other colored area to the center. The user's objective is to manipulate the probe so as to center both ellipses (in the UI) into a single central point with Asymmetry X/Y=0 and minimize the red/colored areas in both ellipses. The probe manipulations (swing Y and X) may directly reflect in horizontal and vertical displacements (asymmetry) of a single center. The probe rotation reflects the rotation of the crossed ellipses around the single center. For a more detailed indication of the quality of the detected walls, global indication is more suitable for image optimization in a clinical setting.

Scan Guide PLAX, PSAX Algorithm

In one embodiment, the scan guide tool includes a PLAX, PSAX algorithm that performs image analysis to detect physiological landmarks. In PLAX view: RV, Septum, Lateral wall, Aortic and MV leaflets. In PSAX view: LV wall, papillary muscles and MV leaflets. In some embodiments, the geometry of the PLAX and PSAX view determines the correctness and the percentage of walls detected determines the quality score.

In some embodiments, the scan guide may include algorithms to perform the following:
1. Image analysis to detect physiological Landmarks:
   LV Walls
   MV leaflets
   LVOT
2. Landmarks analysis that allows view classification and detailed quality estimation:
   LV Walls:
      Percentage of detected left (RV free) wall & middle wall pattern
      discriminate 2ch/4ch views
      Lengths and positions define IQ and LV visibility
      Wall inclinations determine LV rotation
   LVOT+Walls:
      Detected LVOT for good PLAX
   MV leaflets:
      MV position defines scan depth
      MV leaflets determine good PLAX
      LA analysis of image below MV

Parasternal Guide for XY Probe

The scan navigation provided by embodiments is of particular importance for the XY probe due to the unique image presentation and the lack of experience among technicians of its use. For good imaging in both planes, the orientation of the XY probe is different from that of the single plane probe. In one embodiment, simultaneous analysis of scan planes and a simple graphical interface allows intuitive operation of the probe to obtain correct PLAX and PSAX images with good IQ.

XY Probe Orientation

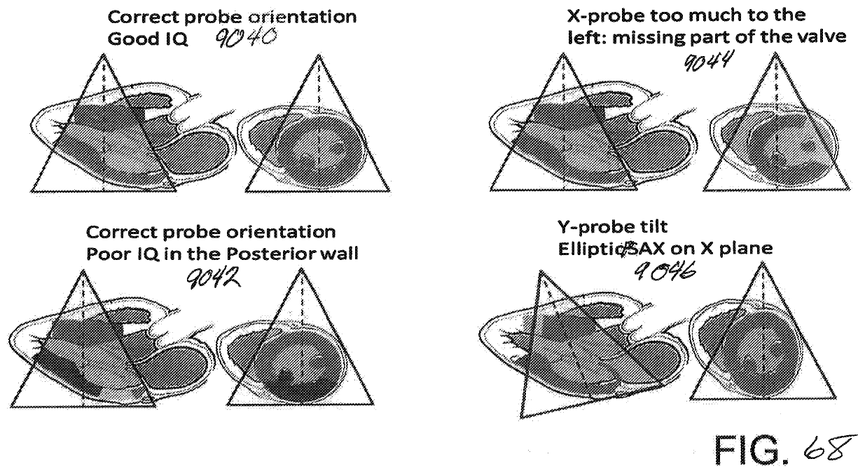

Conventional PLAX images on Y-plane are not optimal for PSAX image on X-plane: the LV ring is elliptic 9022; when the Y-plane image is optimal, the LV is circular (round) 9024. A preferred PSAX image on the X-plane can correspond to oblique PLAX image on the Y-plane. See FIG. 67 for example.

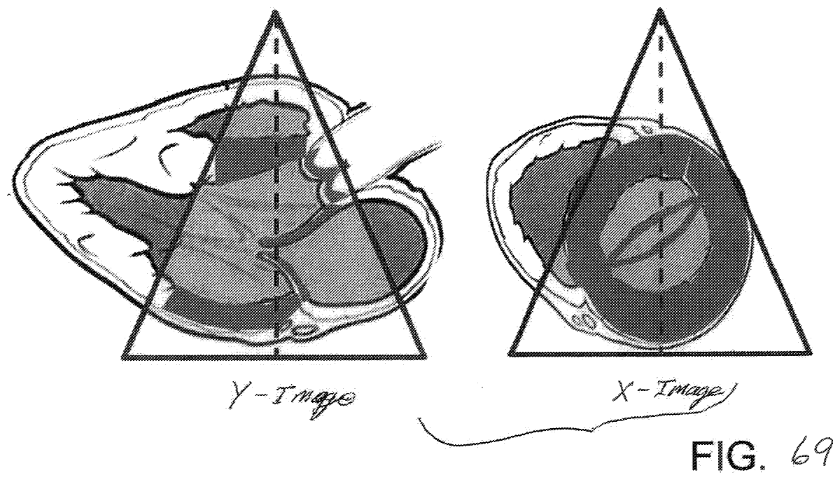

In one embodiment, a transducer probe actuator can be used to control the tilting of the probe with feedback provided via the UI as depicted in FIG. 68. For example, in an exemplary embodiment, a voice coil magnet actuator Model #: NCC01-04-001-1X can be used to control the movement of the probe such as the tilting motion. The actuator, in this example, has a DC load of 1.5 ohm, and can provide a force proportional to the electric current applied to the actuator. The control signal can be calibrated, for example, such that for every ampere of current applied to this actuator, it provides a force of 0.45 Newton. The actuator can provide a maximum continuous force of 0.27 Newton at 0.6 Ampere in this example. In an embodiment, the variable actuator current can be controlled by a programmable digital to analog (DAC) between zero volts and 1 volt, and a unity gain amplifier with output current rating of 1A. At 1V, the output current is 1V/1.5 ohm=0.667 A.

In some embodiments, the tilting angle may be controlled by programming different voltages for the three actuators as previously described. The transducer can also be controlled by adjusting for translation and rotation of the transducer array within a certain range of motion to correct for smaller changes in position of the probe that is mounted on the skin. Note that the pulse rate for ultrasound signal transmission can be regulated by the sensed breathing cycle of the patient to minimize the need for larger adjustments while the patient breaths. The system can also be programmed to select acquired images for quantitative analysis based on the preferred positioning of the transducer. Thus, images 9040 would be used for example, whereas, images 9042 (moved towards the posterior wall), 9044 (translated to a position missing part of the valve), or 9046 (tilt to elliptical PSAX on the X-plane), as seen in FIG. 68 would not be used in this embodiment.

FIG. 69 depicts Y-probe correct position in an exemplary embodiment: the mitral valve Parasternal Short axis, PSAX-MV on X plane.

As can be seen in FIG. 64, once the probe is guided to place on a patient to acquire a proper 2D PLAX view, the view allows an automatic proper placement of the anatomic M-mode. In some embodiments, from the M-mode images, an automatic border detection algorithm tracks the border of the anterior endocardium of the LV septum and posterior walls.

The maximum LV inner diameter, the maximum distance between the two walls, LVIDd, and the minimum LV diameter, the minimum distance between the two walls, LVIDs, can be continuously measured from the border tracing data. The measured LVIDd and LVIDs can be used to calculate the LV volume at diastole and systole. The calculated LV volumes can then be used to calculate EF automatically and continuously.

It is known that cerebral blood flow provides important regional brain activation functions. Functional TCD, transcranial doppler, has been applied to the study of migraines, stroke recovery, Post-Traumatic Stress Disorder, etc. The difficulty of using ultrasound to do transcranial scanning is that the skull significantly attenuates ultrasound signal; it has been reported that the attenuation of ultrasound by the skull is about 13 dB/cm/MHz. A functional ultrasonic imaging method has been developed that is referred to as functional tissue pulsatility imaging (fTPI). In fTPI, the natural pulsatile motion of tissue due to blood flow is measured over the cardiac and respiratory cycles as a surrogate for blood flow itself. By measuring tissue motion and/or tissue strain (the derivative of motion as a function of depth) rather than blood velocity, the fTPI method can overcome the limitation of low backscatter from blood that limits US access to the skull's acoustic windows. The fTPI technique traditionally utilizes B-mode image data. In embodiments of the present invention, instead of using B-mode imaging to measure tissue motion to derive fTPI images, fTPI imaging can be obtained by using Doppler techniques to generate Tissue Doppler imaging directly to measure brain tissue motion and strain. The present systems and embodiments obtain good fTPI images that truly represent the natural pulsatile motion of tissue due to blood flow by removing common-mode motion of brain tissues detected in tissue Doppler images. The computation algorithms used to remove common-mode motion can include one or more of the following:
1) Calculate the average motion speed in a single scan line by averaging the normalized auto-correlation (unit vector) of each sample in this scan line and then calculating the phase angle of the averaged vector.

In general, the tissue velocity is estimated as the phase of autocorrelation using

$$v = \frac{c}{2} \frac{\frac{arg \{ R ( θ , r ) \}}{2 π}}{{f}_{0} T}$$

where R(θ, r) is the autocorrelation at scan angle θ and sample radius r, f₀ is the center transmit frequency, T is the pulse-to-pulse sampling period, and c is ultrasound speed. Particularly, the autocorrelation is calculated as

$$R ( i , j ) = {{\sum }^{K}}_{k = 2} z ( i , j , k ) {z}^{*} ( i , j , k - 1 )$$

where z is the analytic signal or quadrature demodulated signal index by scan line i and sample j, and K indicates the number of frames over which the measurement is made.

The common-mode motion is assumed to be the same amount and direction movement at every position. So it may have different angle for different scan line however the sample for the same scan line. In other words, the Doppler shift caused by common motion in a specific scan line is a constant. So, the average of the phase shift of a scan line can be an estimate of such motion.

$${v}_{0} ( i ) = \frac{c}{2} \frac{arg \{ \frac{1}{M} {\sum }_{j = 1}^{M} \frac{1}{‖ R ( i , j ) ‖} R ( i , j ) \}}{2 π {f}_{0} T}$$

2) Fitting the phase angle across scan lines with a 3^{rd} order polynomial.

To get even more accurate common-mode motion estimation, the final common-mode motion can be fitted as a 3^{rd} order polynomial:

$${\tilde{v}}_{0} ( i ) = {C}_{3} {i}^{3} + {C}_{2} {i}^{2} + {C}_{1} i + {C}_{0}$$

$$⌊ \begin{matrix}{C}_{0} \\ {C}_{1} \\ {C}_{2} \\ {C}_{3}\end{matrix} ⌋ = {( {H}^{T} H )}^{- 1} {H}^{T} ⌊ \begin{matrix}{V}_{0} ( 1 ) \\ {V}_{0} ( 2 ) \\ ⋮ \\ {V}_{0} ( M )\end{matrix} ⌋$$

$$H = {[ \begin{matrix}1 & 1 & … & 1 \\ 1 & 2 & … & M \\ 1 & 4 & … & {M}^{2} \\ 1 & 8 & … & {M}^{3}\end{matrix} ]}^{T}$$

where M is the number of scan lines.
3) Backward phase shift the autocorrelation in the samples of each scan line with the phase angle from the fitted polynomial.

The common-mode motion free autocorrelation is further calculated as

$$\tilde{R} ( i , j ) = R ( i , j ) {e}^{- 2 π {f}_{0} T {\tilde{v}}_{0} ( i ) \frac{2}{c}}$$

4) The common-mode motion free tissue velocity is calculated as

$$v ( θ , r ) = \frac{c}{2} \frac{arg \{ {\sum }_{i = θ - \frac{I}{2}}^{θ + \frac{I}{2}} {\sum }_{j = r - \frac{J}{2}}^{r + \frac{J}{2}} \tilde{R} ( i , j ) \}}{2 π {f}_{0} T}$$

where I and J indicate the number of depth and scan lines, respectively, over which the measurement is made.

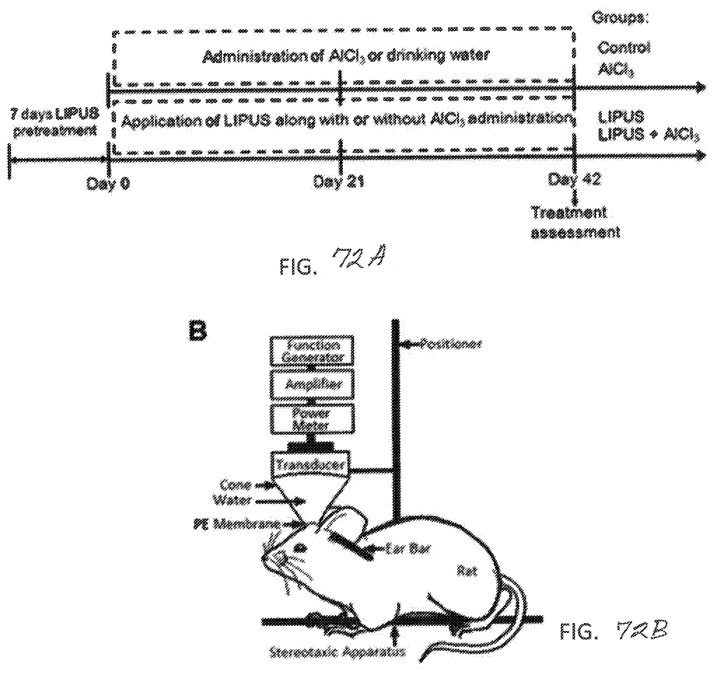

It has been reported that Low-Intensity Pulsed Ultrasound (LIPUS) can stimulate electrical activity in neurons by activating sodium- and calcium-gated channels. See, for example, Tyler, W. J. et al. “Remote excitation of neuronal circuits using low-intensity, low frequency ultrasound.” PLoS One 3, e3511 (2008), and Tufail, Y. et al. “Transcranial pulsed ultrasound stimulates intact brain circuits.” Neuron 66, 681-94 (2010). LIPUS is capable of stimulating neuronal circuits in the brain and promoting levels of brain derived neurotrophic factor (BDNF) which can regulate long term memory. See, for example, the publication of Bekinschtein, P. et al., “BDNF is essential to promote persistence of long-term memory storage”, Proc Natl Acad Sci. USA 105, 2711-6 (2008). Additionally, therapeutic focused-ultrasound to the hippocampus has been reported to exert neuroprotective effects on dementia. As reported by Kumiko Eguchi, et al, “Whole-brain low-intensity pulsed ultrasound therapy markedly improve cognitive dysfunctions in mouse models of dementia—Crucial roles of endothelial nitric oxide synthase”, Brain Stimulation 11 (2018) 959-973), LIPUS therapy ameliorates cognitive dysfunctions, reduces microgliosis along with eNOS upregulation and reduces beta-Amyloid (Aβ) plaque in the Alzheimer's disease, AD, model. This clinical study demonstrated that LIPUS induced a significant increase in the levels of brain-derived neurotrophic factor, BDNF, in hippocampus. In another study, protective effects of low-intensity pulsed ultrasound on aluminum-induced cerebral damage in Alzheimer's disease rat model has been reported by Lin W T, Chen R C, Lu W W, Liu S H, Yang F Y., “Protective effects of low-intensity pulsed ultrasound on aluminum-induced cerebral damage in Alzheimer's disease rat model”, Scientific Reports, 2015; 5:9671. The measurement is shown in FIGS. 72A and 72B. The LIPUS signal was generated by a 1-MHz focused piezoelectric transducer with 50 ms burst lengths at a 5% duty cycle and a repetition frequency of 1 Hz. This study demonstrated a significant improvement in memory retention and attenuated acetylcholinesterase (AChE) activity levels, and beta-Amyloid (Aβ) deposition produced by LIPUS lend support to the author's hypothesis that transcranial LIPUS has a neuroprotective effect against Al-induced cerebral damage and LIPUS can noninvasively promote the levels of neurotrophic factors in the rat hippocampus. In addition, it has been reported that the clearance of potential neurotoxins from the brain would be decreased due to neurovascular reductions in neurodegenerative diseases. See, for example, Bailey, T. L., Rivara, C. B., Rocher, A. B. & Hof, P. R., “The nature and effects of cortical microvascular pathology in aging and Alzheimer's disease”, Neurol Res 26, 573-8 (2004), and also Wu, Z. et al., “Role of the MEOX2 homeobox gene in neurovascular dysfunction in Alzheimer disease”, Nat Med 11, 959-65 (2005). It is important to note that, typical LIPUS intensity, I_{SPTA}, used in these studies is sufficient to promote neurotrophic factors within the ultrasound intensity limits (I_{SPTA} 720 mW/cm²) set by the United States Food and Drug Administration for diagnostic imaging.

The wearable XY-probe is a tool that can be used to evaluate the effect of LIPUS on brain-derived neurotrophic factor in animal model with Alzheimer disease or dementia. Once the animal study has been validated, eventually, the tool can be used to treat Alzheimer disease or dementia in humans. The XY-probe offers electronic steerable Transmit beamforming capability, allows the transmit acoustic energy focused in the region of interest and implements targeted treatment. Due to the one-dimensional construction of each array, for the X-array, the acoustic energy is more confined in the YZ-plane; similarly, for the Y-array, the acoustic energy is more confined in the XZ-plane, see FIGS. 29A-C and also in the application of this probe to cardiac imaging in FIGS. 60-69. When a LIPUS pulse train is used to excite a 3D brain structure, it is preferable to use a 3D acoustic energy field, and the XY-probe can generate the acoustic field in both XZ and YZ planes, a derived 3D field. In summary, the XY-probe can be used to generate LIPUS to excite the whole brain, or to generate focused LIPUS to excite targeted regions of interest for treatment. A further advantage of using XY-probe is to implement LIPUS for improving memory retention, treating Alzheimer, dementia diseases, etc, where the same device can be used in transcranial ultrasound imaging mode to scan the brain, select the targeted treatment area and then use the same XY-probe for implementing the LIPUS treatment procedure.

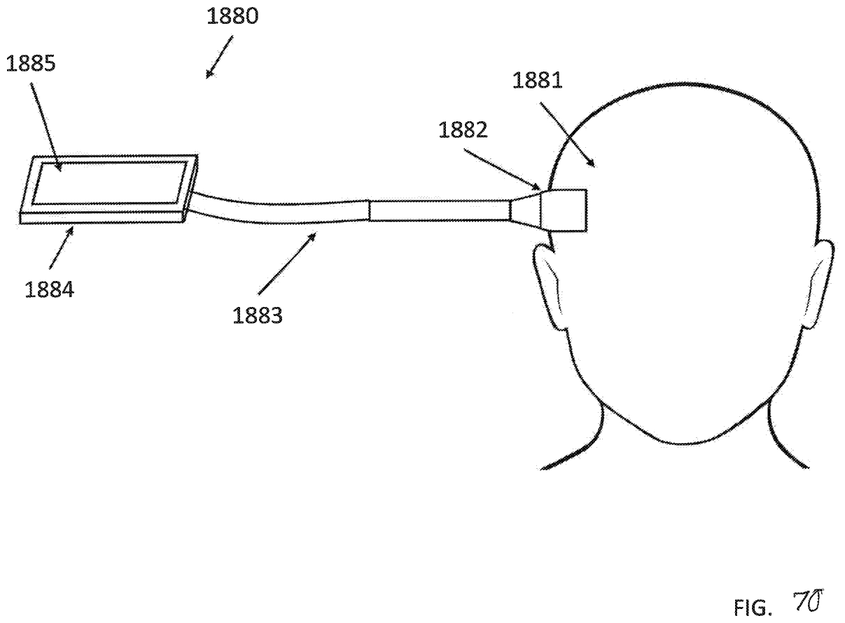

FIG. 70 illustrates a transcranial imaging or therapy operation being performed by the portable transcranial ultrasound system 1880 in accordance with some embodiments described herein. A transducer 1882 is placed at a portion of a patient's cranium 1881 to transmit and receive transcranial ultrasound waves. Signals from the transducer 1882 are transmitted by a wired or wireless connection 1883 to a housing 1884 including a computer and other components as described above with respect to previous embodiments. The housing 1884 can be a housing for a tablet or laptop in some embodiments and can include a display 1885 (such as a touchscreen display) in some embodiments. The computer can process the received ultrasound signals to provide tissue motion or tissue strain measurements. The computer can process the received ultrasound signals to remove common-mode motion from the tissue motion or tissue strain measurements as described above. The computer can control the transducer 1882 to provide therapeutic ultrasound signals such as with LIPUS as described above. In various embodiments, the transducer 1882 can include a one-dimensional array of ultrasound emission and receiving elements, a two-dimensional array of elements, or a biplane array of elements. In some embodiments, the transcranial imaging or therapy operation can be performed through relatively thinner portions of the cranium such as through the portion of the cranium near the temples of the patient (i.e., near the juncture of the sphenoid bone and the frontal bone of the cranium).

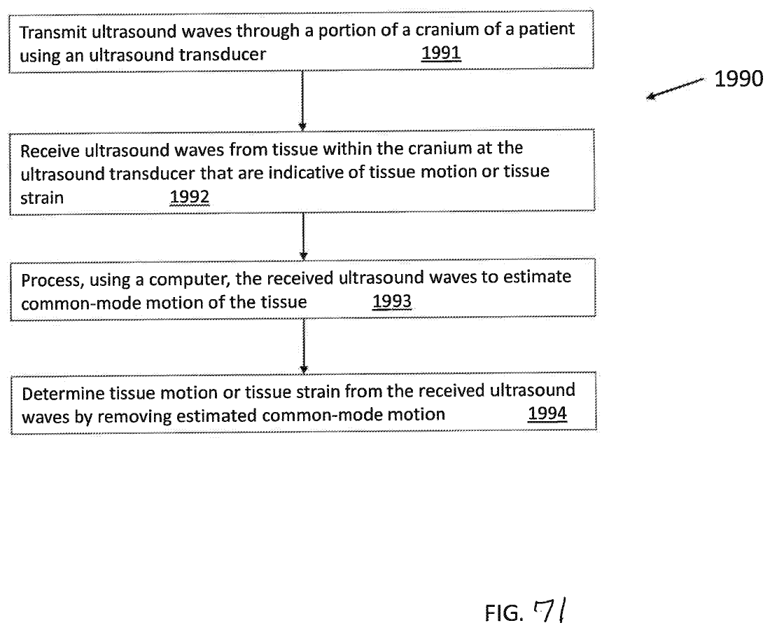

FIG. 71 illustrates a flowchart for a method 1990 of transcranial imaging or therapy in accordance with embodiments taught herein. The method 1990 includes transmitting ultrasound waves through a portion of a cranium of a patient using an ultrasound transducer (step 1991). The method 1990 includes receiving ultrasound waves from tissue within the cranium at the ultrasound transducer that are indicative of tissue motion or tissue strain (step 1992). The method 1990 includes processing, using a computer, the received ultrasound waves to estimate common-mode motion of the tissue (step 1993). The method 1990 includes determining tissue motion or tissue strain from the received ultrasound waves by removing estimated common-mode motion (step 1994).

A system design that can simultaneously drive two XY biplane probes and provide four synchronized images with each probe generating two orthogonal plane images is described generally herein. With this implementation, the two XY-probes can be used simultaneously to do transcranial scanning on two different brain locations. Each probe can generate two orthogonal plane images produced at the scanning location; so, four plane images, two from each scanning location, can be displayed side by side to demonstrate the synchronized brain tissue functions at the two scanning locations. With those images the ‘center of pulsation’ (COP) of brain can be calculated. The synchronized brain tissue functions can be used to detect acute traumatic brain injury (TBI) patients with small and large intracranial bleeds.

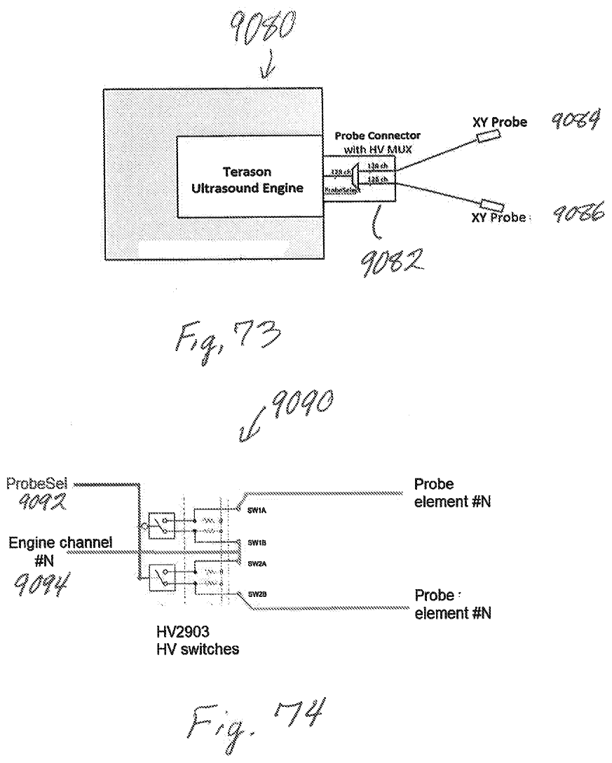

The ultrasound system 9090 can support a single ultrasound transducer, but can be configured to control two probes simultaneously in which probe connector termination board includes a fast switching high voltage multiplexer IC 9082 to connect two XY probe transducer arrays 9084, 9086 as shown in FIG. 73.

The high voltage multiplexer chip 9082 is controlled by a digital signal from the control processor of system 9080 as described previously and can switch between the two probes in less than 5 microseconds. The fast-switching speed allows the software perform imaging across the two XY probe acoustic arrays without noticeable switching delays.

Typically, the transducer cable carries 128 thin coax wires, each coax wire is connected to one element in the acoustic module in the transducer handle and is terminated (soldered) to the printed circuit board. The 160-pin probe connector on the termination PCB mates with a corresponding connector in the control processing board. The 160-pin connector carries the 128 transducer element signals, as well as power supply and ground pins, and several general purpose programmable digital control signals.

For driving two XY-probes simultaneously, the termination PCB has mounted thereon the transducer multiplexing circuits 9086, and connects to two XY transducer cables. The control processor board has 128 high voltage pulser/receiver channels, plus several general purpose digital control signals, one of which is designated as “ProbeSel”, to select between the first XY probe 9084 and the second XY probe 9086 as shown in FIG. 74.

The 128 elements of probe 9084 is connected to the ultrasound processor board 128 channels when the ProbeSel signal 9082 is low, and the 128 elements of probe 9086 is connected to the ultrasound processor board 128 channels when ProbeSel signal 9082 is high. The multiplexing between probe 9084 and probe 9086 is implemented using the high voltage switch IC, such as the commercially available MicroChip HV2903 specification (Chandler, Arizona)(8 chips each configured as 16 single pole double throw switches). The HV2903 switch can handle ultrasound pulse voltage up to 200 volt peak-to-peak, and has a fast switch On/Off time of 5 micro-seconds maximum.

The one Channel high voltage HV2903 mux circuit design to select one element from either probe 9084 or probe 9086 based on the ProbeSel programming configuration. Each of the HV2903 chips has sixteen of these circuits. Thus, 128 channels requires 8 HV2903 chips. The fast switch On/Off time enables the ultrasound imaging operation to switch between the two XY probes on a scan line to scan line basis without degradation of frame rate.

This system provides a point-of-injury/point-of-need/prehospital triage and monitoring device for acute traumatic brain injury (TBI) casualties. Our device will enable early identification of injured military members who have life-threatening yet potentially treatable TBI under prolonged field care (PFC) scenarios. This improves decision-making timelines under remote operating environment scenarios.

The resulting diagnostic ultrasound images of the natural pulsation of brain tissue created by pulsatile blood flow from the heart into structural tissue pulsatility images (sTPI). With those images we calculate the ‘center of pulsation’ (COP) of brain, measured within 1.08+/−0.6 mm (mean+/−standard error) of the midline of ten healthy people. For TBI patients with small intracranial bleeds, the COP measurements yielded a quantitative estimate for midlineshift (MLS) that compared favorably to radiologically determined MILS via CT (R\hat{ }2=0.617). The system can determine for TBI patients with small and large intracranial bleeds, the diagnostic utility of a triage metric (COP+GCS) based upon COP plus Glascow Coma Score (GCS). Here COP can replace CT imaging for midline shift, not available under PFC scenarios, while GCS score is available to those under PFC scenarios. Specifically, the system provides diagnostic utility of a triage process refined retrospectively then tested prospectively while deployed on tablet-sized ultrasound device, one easily deployable in PFC scenarios. TBI patients can be assessed as they enter emergency medicine departments, for example, and before they receive treatment. This system provides diagnostic ultrasound images measures of the

It is noted that the operations described herein are purely exemplary, and imply no particular order. Further, the operations can be used in any sequence, when appropriate, and/or can be partially used. Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than shown.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified.

With the above illustrative embodiments in mind, it should be understood that such embodiments can employ various computer-implemented operations involving data transferred or stored in computer systems. Such operations are those requiring physical manipulation of physical quantities. Typically, though not necessarily, such quantities take the form of electrical, magnetic, and/or optical signals capable of being stored, transferred, combined, compared, and/or otherwise manipulated.

Further, any of the operations described herein that form part of the illustrative embodiments are useful machine operations. The illustrative embodiments also relate to a device or an apparatus for performing such operations. The apparatus can be specially constructed for the required purpose, or can incorporate general-purpose computer devices selectively activated or configured by a computer program stored in the computer. In particular, various general-purpose machines employing one or more processors coupled to one or more computer readable media can be used with computer programs written in accordance with the teachings disclosed herein, or it may be more convenient to construct a more specialized apparatus to perform the required operations.

The foregoing description has been directed to particular illustrative embodiments of this disclosure. It will be apparent, however, that other variations and modifications may be made to the described embodiments, with the attainment of some or all of their associated advantages. Moreover, the procedures, processes, and/or modules described herein may be implemented in hardware, software, embodied as a computer-readable medium having program instructions, firmware, or a combination thereof. For example, one or more of the functions described herein may be performed by a processor executing program instructions out of a memory or other storage device.

It will be appreciated by those skilled in the art that modifications to and variations of the above-described systems and methods may be made without departing from the inventive concepts disclosed herein. Accordingly, the disclosure should not be viewed as limited except as by the scope and spirit of the appended claims.

1. A portable medical ultrasound device comprising:
a transducer probe housing including a transducer array for generating transcranial ultrasound signals;
a portable housing, the housing having a computer in the housing, the computer including at least one processor and at least one memory, and a display, the processor controlling transmission of an ultrasound signal through a cranium; and
an ultrasound beamformer processing circuit that transmits ultrasound pulses with the transducer array, the ultrasound beamformer processing circuit being communicably connected to the computer.

2. The device of claim 1, wherein the at least one memory is a core memory, the device further comprising a graphics processor connected to the core memory in the portable housing.

3. The device of claim 1, wherein the transducer array comprises a first biplane transducer array and a second biplane transducer array.

4. The device of claim 1, further comprising a transducer position control device having a plurality of linear actuators to adjust at least a tilt angle within the transducer probe housing.

5. The device of claim 4, further comprising a backplane on which the transducer position control device is mounted.

6. The device of claim 4, wherein the transducer position control device includes at least three contact points to control a central axis beam control direction of the transducer array.

7. The device of claim 1, wherein the display comprises a touchscreen.

8. The device of claim 7, wherein the computer acts in response to an input from the touchscreen display.

9. The device of claim 8, wherein the computer receives the input from the touchscreen display to control an operation of a transducer position control device.

10. The device of claim 8, wherein the input corresponds to a press gesture against the touchscreen display.

11. The device of claim 9, wherein the computer receives a second input from the touchscreen display to manually adjust the tilt angle.

12. The device of claim 1, wherein the transducer array comprises a plurality of transducer arrays, each operated by the ultrasound beamformer processing circuit in the portable housing that comprises a tablet, and a multiplexing circuit to automatically switch between arrays in response to a programmed control signal from a controller.

13. The device of claim 4, wherein the computer is programmed to control the transducer position control device.

14. The device of claim 13, wherein the computer performs at least one measurement on an ultrasound image based at least in part on a first location of a first cursor on the display.

15. The device of claim 7, wherein the computer receives an input from a keyboard control panel or virtual control panel.

16. The device of claim 4, wherein the display shows a first image of an organ and a second image of the organ simultaneously, and wherein the transducer position control device simultaneously actuates a change in both the first image and the second image.

17. The device of claim 4, wherein the transducer position control device comprises a MEMS actuator.

18. The device of claim 1, wherein the processor is programmed to control transmission of a therapeutic ultrasound signal through the cranium.

19. The device of claim 18, wherein the transducer comprises a biplane probe that generates at least one image of the cranium.

20. The device of claim 18, wherein the transducer comprises a biplane probe to generate a focused ultrasound pulse to a selected region of the brain to stimulate neuron transport.
